(19) United States
(12) Patent Application Publication
(10) Pub. No.: US 2007/0078172 A1
(43) Pub. Date: Apr. 5, 2007

(54) MAO-B INHIBITORS USEFUL FOR TREATING OBESITY

(75) Inventors: John Francis McElroy, Wilmington, DE (US); Robert J. Chorvat, West Chester, PA (US); Rajagopalan Parthasarathi, Chennai (IN)

Correspondence Address: VANCE INTELLECTUAL PROPERTY, PC, 5467 HILL TOP STREET, CROZET, VA, 22932-3167, US

(73) Assignee: JENRIN DISCOVERY, West Chester, PA (US)

(21) Appl. No.: 11/424,274

(22) Filed: Jun. 15, 2006

Related U.S. Application Data
(60) Provisional application No. 60/691,323, filed on Jun. 16, 2005.
(60) Provisional application No. 60/798,467, filed on May 8, 2006.

(51) Int. Cl. A61K 31/4412; A61K 31/137; A61K 31/42

(52) U.S. Cl. 514/355; 514/649; 514/646; 514/650; 514/378

(57) ABSTRACT

The invention provides a method of treating obesity, diabetes, and/or cardiometabolic disorders (e.g., hypertension, dyslipidemias, high blood pressure, and insulin resistance) in a mammal by administering to the mammal a therapeutically effective amount of an irreversible MAO-B inhibitor.

### CROSS-REFERENCE TO RELATED APPLICATIONS

This application claims the priority benefit of U.S. Provisional Application No. 60/691,323 filed Jun. 16, 2005, now pending, and U.S. Provisional Application No. 60/798,467, filed May 8, 2006, now pending, which are incorporated herein by reference.

### FIELD OF THE INVENTION

The present invention relates to a method of treating obesity, diabetes, and/or cardiometabolic disorders (e.g., hypertension, dyslipidemias, high blood pressure, and insulin resistance) by administering to the patient a therapeutically effective amount of a MAO-B inhibitor.

### BACKGROUND OF THE INVENTION

L-Selegiline is a monoamine oxidase (MAO) inhibitor that was developed for the treatment of neurological disorders and is primarily used to treat Parkinson's disease. MAO is an enzyme responsible for metabolizing biogenic monoamines including serotonin, dopamine, histamine, and phenylethylamine. By inhibiting MAO located in the central nervous system (CNS), MAO inhibitors increase the concentration of monoamines present within the brain synapses. This enhances monoamine-mediated neurotransmission, effectively treating neurological and psychiatric disorders such as Parkinson's disease and depression. Currently, the only approved clinical use of L-selegiline and other MAO inhibitors is for the treatment of CNS disorders such as Parkinson's disease and depression.

Obesity is associated with an increase in the overall amount of adipose tissue (i.e., body fat), especially adipose tissue localized in the abdominal area. Obesity has reached epidemic proportions in the United States. The prevalence of obesity has steadily increased over the years among all racial and ethnic groups. According to the United States Surgeon General, 61% of the adult population and 14% of children are obese or overweight. Forty four million Americans are obese, with an additional eighty million deemed medically overweight. Obesity is responsible for more than 300,000 deaths annually, and is one of the leading causes of preventable death in the United States. Obesity is a chronic disease that contributes directly to numerous dangerous co-morbidities, including type 2 diabetes, cardiovascular disease, inflammatory diseases, premature aging, and some forms of cancer. Type 2 diabetes, a serious and life-threatening disorder with growing prevalence in both adult and childhood populations, is currently the 7^{th} leading cause of death in the United States. Since more than 80% of patients with type 2 diabetes are overweight, obesity is the greatest risk factor for developing type 2 diabetes. Increasing clinical evidence indicates that the best way to control type 2 diabetes is to reduce weight.

The most popular over-the counter drugs for the treatment of obesity, phenylpropanolamine and ephedrine, and the most popular prescription drug, fenfluramine, were removed from the marketplace as a result of safety concerns. Drugs currently approved for the long-term treatment of obesity fall into two categories: (a) Central Nervous System (CNS) appetite suppressants such as sibutramine and (b) gut lipase inhibitors such as orlistat. CNS appetite suppressants reduce eating behavior through activation of the ‘satiety center’ in the brain and/or by inhibition of the ‘hunger center’ in the brain. Gut lipase inhibitors reduce the absorption of dietary fat from the gastrointestinal (GI) tract. Although sibutramine and orlistat work through very different mechanisms, they share in common the same overall goal of reducing body weight secondary to reducing the amount of calories that reach the systemic circulation. Unfortunately, these indirect therapies produce only a modest initial weight loss (approximately 5% compared to placebo) that is usually not maintained. After one or two years of treatment, most patients return to or exceed their starting weight. In addition, most approved anti-obesity therapeutics produce undesirable and often dangerous side effects that can complicate treatment and interfere with a patient's quality of life.

The lack of therapeutic effectiveness, coupled with the spiraling obesity epidemic, positions the ‘treatment of obesity’ as one of the largest and most urgent unmet medical needs. There is, therefore, a real and continuing need for the development of improved medications that treat or prevent obesity.

MAO-B inhibitors, such as L-selegiline, have been clinically useful in the treatment of CNS disorders. They have now unexpectedly been discovered to also have anti-obesity activity. This new discovery provides a novel approach for the prevention or treatment of obesity.

### SUMMARY OF THE INVENTION

In an aspect, the present invention provides novel methods for treating obesity, diabetes, and/or cardiometabolic disorders (e.g., hypertension, dyslipidemias, high blood pressure, and insulin resistance), comprising: administering to a patient in need of such treatment a therapeutically effective amount of at least one of the compounds of the present invention or a pharmaceutically acceptable salt form thereof.

In another aspect, the present invention provides a method of treating a co-morbidity of obesity by administering a MAO-B inhibitor.

In another aspect, the present invention provides a method of preventing or reversing the deposition of adipose tissue in a patient in need thereof by administering a MAO-B inhibitor.

In another aspect, the present invention provides the use of compounds of the present invention for the manufacture of a medicament for the treatment of obesity, diabetes, and/or cardiometabolic disorders.

These and other aspects, which will become clear during the following detailed description, have been achieved by the inventors' surprising discovery that a MAO-B inhibitor was effective in reducing the amount of adipose tissue in a mammal.

### DETAILED DESCRIPTION OF THE INVENTION

The present invention is based on the unexpected finding that a MAO-B inhibitor is capable of reducing the amount of adipose tissue (i.e., body fat) in a warm-blooded mammal. This finding was unexpected because body fat can be reduced despite little, if any, concomitant reduction in food intake.

Thus, the present invention provides a novel method for treating a disease, comprising: administering to a patient in need thereof a therapeutically effective amount of a compound of the present invention or a pharmaceutically acceptable salt form thereof, wherein the disease is selected from obesity, diabetes, cardiometabolic disorders, and a combination thereof. By preventing or reversing the deposition of adipose tissue, MAO-B inhibitors are expected to reduce the incidence or severity of obesity, diabetes, cardiometabolic disorders, thereby also reducing the incidence or severity of associated co-morbidities. The present invention, consequently, also provides a method of treating obesity co-morbidities, which include diabetes, Metabolic Syndrome, dementia, cancer, and heart disease.

In another embodiment, the cardiometabolic disorder is selected from hypertension, dyslipidemias (e.g., undesirable blood lipid levels, elevated cholesterol levels, and lowered LDL levels), high blood pressure, and insulin resistance.

In another embodiment, the co-morbidity is selected from hypertension; gallbladder disease; gastrointestinal disorders; menstrual irregularities; degenerative arthritis; venous statis ulcers; pulmonary hypoventilation syndrome; sleep apnea; snoring; coronary artery disease; arterial sclerotic disease; pseudotumor cerebri; accident proneness; increased risks with surgeries; osteoarthritis; high cholesterol; and, increased incidence of malignancies of the ovaries, cervix, uterus, breasts, prostrate, and gallbladder.

In another embodiment, the present invention also provides a method of preventing or reversing the deposition of adipose tissue in a patient in need thereof by the administration to a mammal in need thereof a therapeutically effective amount of a MAO-B inhibitor.

[1] In another embodiment, the MAO-B inhibitor is selected from: L-selegiline; desmethylselegiline; Rasagiline; Pargyline; Lazabemide; R0-16-6491, AGN 1135; MDL 72,974; MDL 72,145; MDL 72,638; LY 54761; MD 780236; Iproniazid; Phenelzine; Nialamide; Phenylhydrazine; 1-Phenylcyclopropylamine; Isocarboxazid; Tranylcypromine; and EVT 301, or a pharmaceutically acceptable salt thereof.

[2] In another embodiment, the inhibitor is selected from: L-selegiline; Rasagiline; Lazabemide; and Pargyline, or a pharmaceutically acceptable salt thereof.

[3] In another embodiment, the inhibitor is L-selegiline, or a pharmaceutically acceptable salt thereof.

[4] In another embodiment, the inhibitor is Rasagiline, or a pharmaceutically acceptable salt thereof.

[5] In another embodiment, the inhibitor is Lazabemide, or a pharmaceutically acceptable salt thereof

[6] In another embodiment, the inhibitor is Pargyline, or a pharmaceutically acceptable salt thereof.

Additional MAO-B inhibitors useful in the present invention are described below. The contents of the patents and publications discussed herein are incorporated herein by reference.

[7] In another embodiment, the MAO-B inhibitor is selected from the group of compounds described in The European Journal of Medicinal Chemistry 23, 441 (1988) and The Journal of Medicinal Chemistry 36, 1157 (1993) or pharmaceutically acceptable salts thereof. Compounds from these publications include compounds of formula I or pharmaceutically acceptable salts thereof:

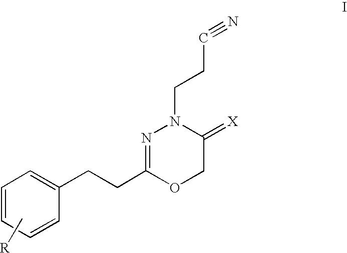

wherein:

X is O or S; and,

R is selected from H, or 3-Me, 4-Me, 3-Cl, 4-Cl, 3-OMe, 4-OMe, 3-NO₂, and 4-NO₂.

[8] In another embodiment, in formula I, X is O, and R is selected from H, 3-OMe, 4-OMe, 3-Me, and 3-Cl.

[9] In another embodiment, the MAO-B inhibitor is selected from the group of compounds described in The Journal of Medicinal Chemistry 36, 1157 (1993) and U.S. Pat. Nos. 5,250,551 and 5,376,648 or pharmaceutically acceptable salts thereof. Compounds from this publication includes compounds of formula II or IIa or pharmaceutically acceptable salts thereof:

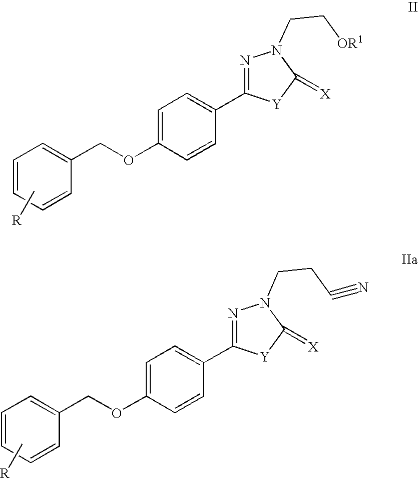

wherein:

X and Y are independently selected from O and S;

R is selected from H, CF₃, halogen, Me, NO₂, and OMe; and,

R¹ is selected from H and C₁₋₄ alkyl.

[10] In another embodiment, in formula II or IIa:

X and Y are each O; and,

R is selected from H, 3-OMe, 4-OMe, 3-Me, 4-Me, 3-Cl, and 4-Cl.

[11] In another embodiment, in formula II or IIa:

X is S;

Y is O; and,

R is H.

[12] In another embodiment, the MAO-B inhibitor is selected from the group of compounds described in U.S. Pat. No. 5,525,619 or pharmaceutically acceptable salts thereof. Compounds from this publication include compounds of formula III or pharmaceutically acceptable salts thereof:

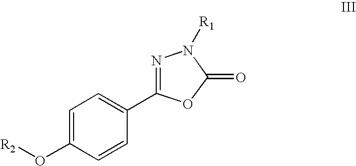

wherein:

R₁ is selected from H and a C₁₋₄ alkyl group, which is substituted with one or more groups selected from OH, phenoxy, C₁₄ alkoxy, C₁₋₄ alkylthio, SH, C₁₋₄ alkoxy-C₁₋₄ alkoxy, di-C₁₋₄ alkylamino, N-C₁₋₄ alkyl-N-propynylamino, and a C₃₋₄ alkynyl; and,

R₂ is a C₁₋₈ alkyl group, which is substituted with one or more groups selected from halogen, OH, 1-imidazolyl, 3-tetrahydropyranyl, and trifluoro-C₃₋₅-alkenyl.

[13] Examples of compounds of formula III include:
5-[4-(4,4,4-trifluorobutoxy)phenyl]-3-methoxyethyl- 1,3,4-oxadiazol-2(3H)-one;
5-[4-(4,4,4-trifluorobutoxy)phenyl]-3-hydroxyethyl- 1,3,4-oxadiazol-2(3H)-one;
5-[4-(4,4,4-trifluorobutoxy)phenyl]-3-methylthioethyl-1,3,4-oxadiazol-2(3H)-one;
5-[4-(4,4,4-trifluoro-2-butenyloxy)phenyl]-3-methoxyethyl-1,3,4-oxadiazol-2(3H)-one;
5-[4-(4,4,4-trifluoro-3(R)-hydroxybutoxy)phenyl]-3-methoxyethyl- 1,3,4-oxadiazol-2(3H) -one; and,
5-[4-(tetrahydropyran-3-ylmethoxy)phenyl]-3-methoxyethyl-1,3,4-oxadiazol-2 (3H)-one, or pharmaceutically acceptable salts thereof.

[14] In another embodiment, the MAO-B inhibitor is selected from the group of compounds described in Bioorganic Medicinal Chemistry letters 4, 1195(1994) and in U.S. Pat; Nos.: 5,073,563; 5,100,914; and, 5,227,392 or pharmaceutically acceptable salts thereof. Compounds from these documents include those of formula IV or pharmaceutically acceptable salts thereof:

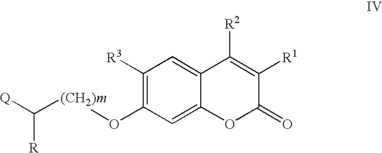

wherein:

Q is phenyl substituted with 0-3 groups selected from halogen, C₁₋₆ alkyl, and C₁₋₆ alkoxy;

alternatively, Q is phenyl substituted with one group selected from NO₂, —CN, and trifluoromethyl;

alternatively, Q is selected from a naphthyl ring and 5-10 membered heteroaryl consisting of carbon atoms and 1-3 heteroatoms selected from O, N, and S(O)ₚ, wherein the naphthyl and heteroaryl are substituted with 0-2 groups selected from halogen, C₁₋₆ alkyl, C₁₋₆ alkoxy, C₁₋₄ alkoxy-C₁₋₄ alkylene, C₃₋₈ cycloalkyl, C₁₋₄ alkyl-C₃₋₈ cycloalkylene, benzyl, and 5-6 membered heteroaryl consisting of carbon atoms and 1-3 heteroatoms selected from O, N, and S(O)ₚ,

R is selected from H and C₁₋₆ alkyl;

R¹ is selected from H, halogen, and C₁₋₆ alkyl;

R² is selected from H, halogen, and C₁₋₆ alkyl;

R³ is selected from H, halogen, and C₁₋₆ alkyl;

m is selected from 0, 1, 2, 3, and 4; and,

p is selected from 0, 1, and 2.

[15] Examples of compounds of formula IV include those wherein:

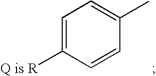

R is selected from H, Me, and isopropyl;

alternatively, Q is selected from the following: 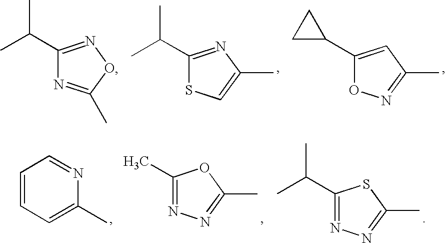
[16] Examples of compounds of formula IV include:
3,4-Dimethyl-7-(4-isopropylphenyl)-methoxycoumarin;
3,4-Dimethyl-7-(2-naphthyl)-methoxycoumarin;
7-(4-tert-Butylphenyl)-methoxy-3,4-dimethylcoumarin;
3,4-Dimethyl-7-(2-methylphenyl)-methoxycoumarin;
3,4-Dimethyl-7-(3-methylphenyl)-methoxycoumarin;
3,4-Dimethyl-7-(4-methylphenyl)-methoxycoumarin;
3,4-Dimethyl-7-(2,5-dimethylphenyl)-methoxycoumarin;
7-(4-Methoxyphenyl)-methoxy-3,4-dimethylcoumarin;
7-(4-Trifluoromethylphenyl)-methoxy-3,4-dimethylcoumarin;
7-(3-Trifluoromethylphenyl)-methoxy-3,4-dimethylcoumarin;
6-Ethyl-3,4-dimethyl-7-(2-phenyl)-ethoxycoumarin;
7-(4-Isopropylphenyl)-methoxycoumarin;
4-Methyl-7-(4-isopropylphenyl)-methoxycoumarin;
3-Methyl-7-(4-isopropylphenyl)-methoxycoumarin;
3-Chloro-4-methyl-7-(4-isopropylphenyl)-methoxycoumarin;
7-(3-Phenylpropoxy)-coumarin;
3,4-Dimethyl-7-(3-phenylpropoxy)-coumarin;
6-Chloro-3,4-dimethyl-7-(4-isopropylphenyl)-methoxycoumarin;
7-(2-Benzylthiazol-4-yl)-methoxy-3,4-dimethylcoumarin;
7-(2-Isopropylthiazol-4-yl)-methoxy-3,4-dimethylcoumarin;
7-(3-Cyclopentylisoxazol-5-yl)-methoxy-3,4-dimethylcoumarin;
7-[3-(1-Methoxyethyl)-isoxazol-5-yl]-methoxy-3,4-dimethylcoumarin;
7-(2-Cyclopropylthiazol-4-yl)-methoxy-3,4-dimethylcoumarin;
6-Ethyl-7-(5-isopropyl-1-methylpyrazol-3-yl)-methoxy-3,4-dimethylcoumarin;
6-Ethyl-3,4-dimethyl-7-(2-methyl-1,3,4-thiadiazol-5-yl)-methoxycoumarin;
7-(3-Cyclohexylisoxazol-5-yl)-methoxy-3,4-dimethylcoumarin;
7-(2-tert-Butylthiophen-5-yl)-methoxy-3,4-dimethylcoumarin;
3,4-Dimethyl-7-(2-cyclopropyl-1,3,4-thiadiazol-5-yl)-methoxycoumarin;
3,4-Dimethyl-7-[3-(1-methylcyclopropyl)-isoxazol-5-yl]-methoxycoumarin;
3,4-Dimethyl-7-[3-(tetrahydrofuran-3-yl)-isoxazol-5-yl]-methoxycoumarin;
3,4-Dimethyl-7-(3-cyclopentylisoxazol-5-yl)-methoxycoumarin;
3,4-Dimethyl-7-(3-cyclohexylisoxazol-5-yl)-methoxycoumarin;
6-Chloro-3,4-dimethyl-7-(2-pyridinyl)-methoxycoumarin;
3,6-Dichloro-4-methyl-7-(5-methyl-1,3,4-thiadiazol-2-yl)-methoxycoumarin; and,
3,6-Dichloro-4-methyl-7-(2-isopropylthiazol-4-yl)-methoxycoumarin, or pharmaceutically acceptable salts thereof.

[17] In another embodiment, the MAO-B inhibitor is selected from the group of compounds described in The Journal of Medicinal Chemistry 38, 4786 (1995) and U.S. Pat. Nos. 5,100,910 and 5,262,432 or pharmaceutically acceptable salts thereof. Compounds from these documents include compounds of formula V and VI or pharmaceutically acceptable salts thereof:

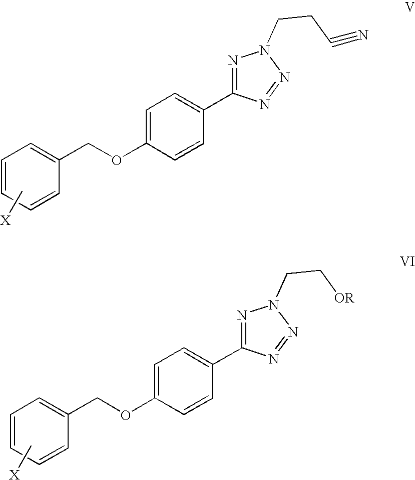

wherein:

X is selected from H, halogen, Me, OMe, CF₃, and phenyl; and,

R is selected from H and C₁₋₄ alkyl.

[18] In another embodiment, the compounds of formula V and VI include:

X is selected from H, 2-Cl, 3-Cl, 4-Cl, 2-F, 3-F, 4-F, 2-Me, 3-Me, 4-Me, 2-CF₃, CF₃, and 4-CF₃.

[19] In another embodiment, the MAO-B inhibitor is selected from the group of compounds described in The Journal of Medicinal Chemistry 43, 1684 (2000) or pharmaceutically acceptable salts thereof. Compounds from this publication include compounds of formula VII or pharmaceutically acceptable salts thereof:

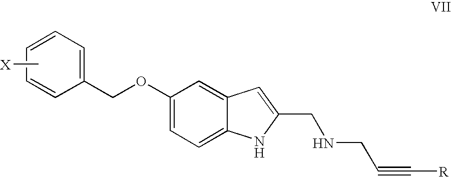

wherein:

R is selected from H and Me; and,

X is selected from H, 2-Cl, 3-Cl, 4-Cl, 2-F, 3-F, 4-F, 2-Me, 3-Me, 4-Me, 2CF₃, 3CF₃, and 4-CF₃.

[20] In another embodiment, the MAO-B inhibitor is selected from the group of compounds described in Bioorganic and Medicinal Chemistry Letters 6, 115 (1996) and U.S. Pat. No. 5,380,755 or pharmaceutically acceptable salts thereof. Compounds from these documents include compounds of formula VIII, IX, and X, or pharmaceutically acceptable salts thereof:

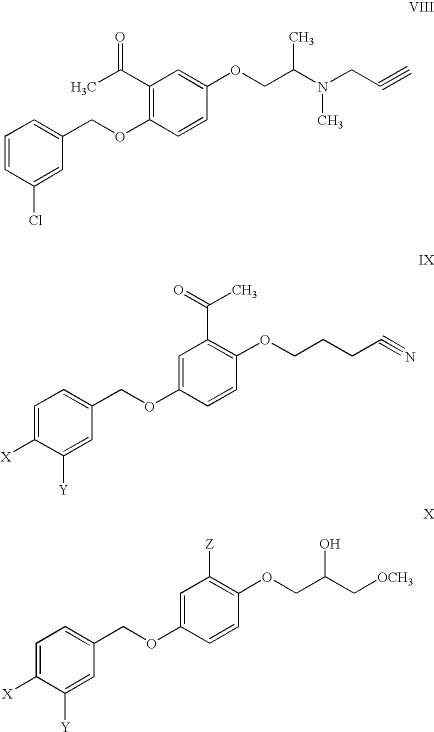

wherein:

X and Y are selected from H, Cl, F, CH₃ and CF₃; and,

Z is selected from —COCH₃ and CHO.

[21] In another embodiment, the MAO-B inhibitor is selected from the group of compounds described U.S. Pat. No. 4,454,158 or pharmaceutically acceptable salts thereof. Compounds from this publication include compounds of formula XI or pharmaceutically acceptable salts thereof:

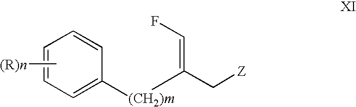

wherein:

R is selected from C₁₋₈ alkyl, Cl-₁₋₈ alkoxy, OH, halogen, CF₃, NO₂, C₁₋₆ alkylcarbonyl, benzoyl, phenyl, 1-naphthyl, 2-naphthyl, 1-indenyl, 2-indenyl, 3-indenyl, 1-fluorenyl, 2-fluorenyl, 9-fluorenyl, 1-piperidinyl, 2-piperidinyl, 3-piperidinyl, 2-pyrrolyl, 3-pyrrolyl; 2-thienyl, 3-thienyl, 2-furanyl, 3-furanyl, 2-indolyl, 3-indolyl, 2-thianaphthenyl, 3-thianaphthenyl, 2-benzofuranyl, and 3-benzofuranyl;

Z is NH₂ or OH;

m is selected from 0, 1, 2, and 3; and,

n is selected from 0, 1, 2, and 3.

[22] Examples of compounds of formula XI include:
(Z)-or (E)-(p-fluorophenethyl)-3-fluoroallylamine,
(Z)-or (E)-2-(2′-methoxy)phenyl-3-fluoroallylamine,
(Z)-or (E)-2-(3′-methoxy)phenyl-3-fluoroallylamine,
(Z)-or (E)-2-(4′-methoxy)phenyl-3-fluoroallylamine,
(Z)-or (E)-2-(3′-hydroxy)phenyl-3-fluoroallylamine,
(Z)-or (E)-N-ethyl 2-(3′-methoxy)phenyl-3-fluoroallylamine,
(Z)-or (E)-2-(3′,4′-dimethoxy)phenyl-3-fluoroallylamine,
(Z)-or (E)-N-ethyl 2-(3′,4′-dimethoxy)phenyl-3-fluoroallylamine,
(Z)-or (E)-2-(4′-chloro)phenyl-3-fluoroallylamine,
(Z)-or (E)-2-(3′-trifluoromethyl)phenyl-3-fluoroallylamine.
(Z)-or (E)-2-(.alpha.-naphthyl)-3-fluoroallylamine,
(Z)-or (E)-2-(.beta.-naphthyl)-3-fluoroallylamine,
(E)-2-(2′-methoxy)phenyl-3-fluoroallyl alcohol,
(E)-2-(3′-methoxy)phenyl-3-fluoroallyl alcohol,
(E)-2-(4′-methoxy)phenyl-3-fluoroallyl alcohol,
(E)-2-(3′,4′-dimethoxy)phenyl-3-fluoroallyl alcohol,
(E)-2-(2′-methoxy)benzyl-3-fluoroallyl alcohol,
(E)-2-(3′-methoxy)benzyl-3-fluoroallyl alcohol,
(E)-2-(4′-methoxy)benzyl-3-fluoroallyl alcohol,
(E)-2-phenyl-3-fluoroallyl alcohol,
(E)-2-benzyl-3-fluoroallyl alcohol,
(E)-2-(3′,4′-dimethoxy)benzyl-3-fluoroallyl alcohol,
Z)-or (E)-2-(3′-methoxyphenyl)-3-fluoroallylamine, and
(Z)-or (E)-2-(3′,4′-dimethoxyphenyl)-3-fluoroallylamine;
   or pharmaceutically acceptable salts thereof.

[23] In another embodiment, the MAO-B inhibitor is selected from the group of compounds described U.S. Pat. No. 4,764,522 or pharmaceutically acceptable salts thereof. Compounds from this publication include compounds of formula XII or pharmaceutically acceptable salts thereof:

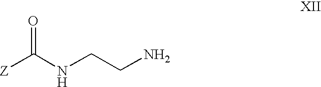

wherein:

Z is aryl or is selected from a 5- or 6-membered heteroaryl shown below:

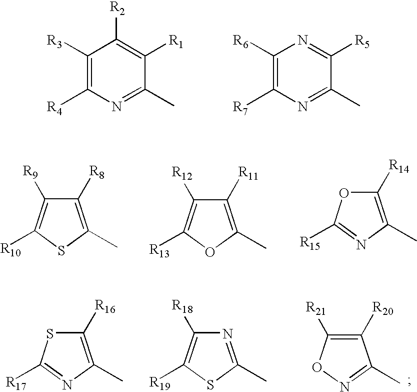

at least two of R₁, R₂, R₃, and R₄ are H and the remaining two are independently selected from H, halogen, NO₂, NH₂, OH, C₁₋₆ alkoxy, C₁₋₆ alkyl, phenyloxy, and phenylmethyloxy, the phenyl group of phenyloxy and phenylmethyloxy being optionally substituted with a group selected from halogen, C₁₋₆ alkyl, C₁₋₆ alkoxy, NO₂, and OH;

R₅, R₆, and R₇ are independently selected from H and halogen;

R₈, R₉ and R₁₀ are independently selected from H, halogen, and C₁₋₆ alkyl, provided that at least one of R₈, R₉, and R₁₀ is other than H;

R₁₁, R₁₂, and R₁₃ are independently selected from H and halogen, provided that at least one of R₁₁, R₁₂ and R₁₃ are other than H;

R₁₄, R₁₅, R₁₆, R₁₈ and R₁₉ are selected from H, halogen, and C₁₋₆ alkyl;

R₁₇ is selected from H and halogen; and,

R₂₀ and R₂₁ are selected from H and C₁₋₆ alkyl.

[24] Examples of compounds of formula XII include:
N-(2-aminoethyl)-4-methoxypyridine-2-carboxamide,
N-(2-aminoethyl)thiazole-2-carboxamide,
N-(2-aminoethyl)-4-bromopyridine-2-carboxamide,
N-(2-aminoethyl)-4-chloropyridine-2-carboxamide,
N-(2-aminoethyl)-2-chlorothiazole-4-carboxamide,
N-(2-aminoethyl)-5-methylisoxazole-3-carboxamide,
N-(2-aminoethyl)-6-bromopyridine-2-carboxamide,
N-(2-aminoethyl)-6-chloropyridine-2-carboxamide,
N-(2-aminoethyl)-5-bromothiazole-4-carboxamide,
N-(2-aminoethyl)-3-aminopyridine-2-carboxamide,
N-(2-aminoethyl)pyridine-2-carboxamide,
N-(2-aminoethyl)-5-chloropyridine-2-carboxamide,
N-(2-aminoethyl)-2-chlorothiazole-4-carboxamide hydrochloride,
N-(2-aminoethyl)-3-aminopyridine-2-carboxamide dihydrochloride,
N-(2-aminoethyl)pyridine-2-carboxamide dihydrochloride, and
N-(2-aminoethyl)-5-chloropyridine-2-carboxamide hydrochloride,
   or pharmaceutically acceptable salts thereof.

[25] In another embodiment, the MAO-B inhibitor is selected from the group of compounds described U.S. Pat. No. 5,238,962 or pharmaceutically acceptable salts thereof. Compounds from this publication include compounds of formula XIII or pharmaceutically acceptable salts thereof:

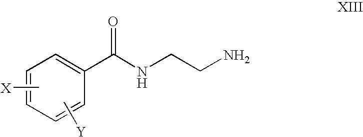

wherein:

X and Y are independently selected from H, Cl, F, Br, C₁₋₆ alkyl, C₁₋₆ alkoxy, CF₃, —CN, sulfamoyl, mono(C₁₋₆ alkyl)sulfamoyl, and di(C₁₋₆ alkyl)sulfamoyl;

provided that Y is other than H when X is 3-Br;

alternatively, X and Y, when on adjacent carbon atoms, together form a methylenedioxy group.

[26] Examples of compounds of formula XIII include:
N-(2-aminoethyl)-p-chlorobenzamide,
N-(2-aminoethyl)-p-fluorobenzamide,
N-(2-aminoethyl)-p-bromobenzamide,
N-(2-aminoethyl)-3,4-dichlorobenzamide, and
N-(2-aminoethyl)-2,4-dichlorobenzamide,
   or pharmaceutically acceptable salts thereof.

[27] In another embodiment, the MAO-B inhibitor is selected from the group of compounds described in U.S. Pat. No. 5,169,868 or pharmaceutically acceptable salts thereof. Compounds from this publication include compounds of formula XIV or pharmaceutically acceptable salts thereof:

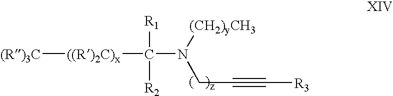

wherein:

R₁ is selected from H and C₁₋₆ alkyl;

R₂ is selected from H and C₁₋₆ alkyl;

R₃ is selected from H and C₁₋₆ alkyl;

R′ is selected from H and halogen;

R″ is selected from H and halogen;

x is selected from 1, 2, 7, 8, 9, 10, 11, 12, and 13;

y is selected from 0, 1, 2, 3, 4, and 5; and,

z is selected from 1, 2, 3, 4, and 5.

[28] Examples of compounds of formula XIV include:
N-(2-propyl)-N-methylpropargylamine-HCl;
N-(2-butyl)-N-methylpropargylamine-HCl;
N-(1-butyl)-N-methylpropargylamine-HCl;
N-(2-heptyl)-N-methylpropargylamine-HCl;
N-(1-heptyl)-N-methylpropargylamine-HCl;
N-(2-pentyl)-N-methylpropargylamine-HCl;
N-(1-pentyl)-N-methylpropargylamine-oxalate;
N-(2-decyl)-N-methylpropargylamine-HCl;
N-(2-dodecyl)-N-methylpropargylamine-HCl; and,
R(−)-N-(2-butyl)-N-methylpropargylamine-oxalate,
   or pharmaceutically acceptable salts thereof.

[29] In another embodiment, the MAO-B inhibitor is selected from the group of compounds described in U.S. Pat. No. 5,326,770 or pharmaceutically acceptable salts thereof. Compounds from this publication include compounds of formula XV or pharmaceutically acceptable salts thereof:

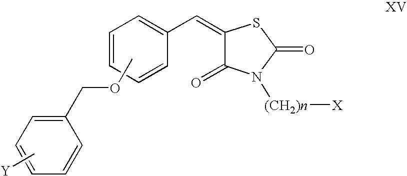

wherein:

X is —CN or —SCN;

Y is selected from H, halogen, C₁₋₆ alkyl, C₁₋₆ alkoxy, and CF₃; and,

n is selected from 1, 2, 3, 4, 5, and 6.

[30] Examples of compounds of formula XV include:
2,4-dioxo-5-[3-(phenylmethoxy)-phenylmethylene]-4-thiazolidinebutanenitrile, and
2,4-dioxo-5-[3-(phenylmethoxy)-phenylmethylene]-4-thiazolidinepentanenitrile,
   or pharmaceutically acceptable salts thereof.

[31] In another embodiment, the MAO-B inhibitor is selected from the group of compounds described U.S. Pat. No. 5,574,055 or pharmaceutically acceptable salts thereof. Compounds from this publication include compounds of formula XVI or pharmaceutically acceptable salts thereof:

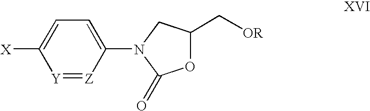

wherein:

R is selected from H and C₁₋₆ alkyl;

X is selected from C₂₋₈ cycloalkenyl, bicyclo[2.2. 1]hept-2-yl optionally substituted by phenyl-2-oxo-5-methoxymethyloxazolidinyl; bicyclo[2.2. 1]-hept-5-en-2-yl; adamantyl; C₃₋₆ cycloalkyl; and piperidinyl;

X is optionally mono-or multiply-substituted by halogen, NH₂, C₁₋₆ alkyl, —CN, O, hydroxyimino, ethylenedioxy, —OR₁, ═CR₂R₃, —(CH₂)ₙR_{4,} —COR₅, and —NR₆R₇;

R₂ is selected from H and C₁₋₆ alkyl;

R₃ is selected from H, —CN, C₁₋₆ alkyl, phenyl, and CO₂—C₁₋₆ alkyl;

R₄ is selected from —CN, NH₂, —NHCOCH₃, —C(O)C₆H₄-halogen, phenyl, and OH;

R₅ is selected from C₁₋₆ alkyl, —CH═CHC₆H₅, —C₆H₄—CF₃, —OC(CH₃)₃, and C₁₋₆ alkoxy;

R₆ is selected from H and COCH₃;

R₇ is selected from COCH₃, benzyl, and —(CH₂)ₙNHCOC₆H₄-halogen; and,

n is selected from 1, 2, and 3.

[32] Examples of compounds of formula XVI include:
(RS)-3-(4-Cyclohexyl-phenyl)-5-hydroxymethyl-oxazolidin-2-one;
(RS)-3-(4-cyclohexylphenyl)-5-methoxymethyl-oxazolidin-2-one;
(R)-3-(4-cyclohexyl-phenyl)-5-methoxymethyl-oxazolidin-2-one;
(RS)-3-[4-(4-oxocyclohexyl)-phenyl]-5-methoxymethyl-oxazolidin-2-one;
(RS)-3-[4-(trans-4-hydroxy-cyclohexyl)-phenyl]-5-methoxymethyl-oxazolidin-2-one;
(RS)-3-[4-(4-hydroxy-imino-cyclohexyl)-phenyl]-5-methoxymethyl-oxazolidin-2-one;
(R)-3-[4-trans-4-hydroxy-cyclohexyl)-phenyl]-5-methoxymethyl-oxazolidin-2-one;
(RS)-3-[4-(trans-4-methoxy-cyclohexyl)-phenyl]-5-methoxymethyl-oxazolidin-2-one;
(R)-3-[4-(4-oxo-cyclohexyl)-phenyl]-5-methoxymethyl-oxazolidin-2-one;
(RS)-3-[trans-4-[4-(5-methoxymethyl-2-oxo-oxazolidin-3-yl)-phenyl]-cyclohexyloxy]-propionitrile;
(RS)-trans-4-[4-(5-methoxymethyl-2-oxo-oxazolidin-3-yl)-phenyl]-cyclohexyl ester;
(RS)-3-[4-(cis- or trans-4-hydroxymethyl-cyclohexyl)-phenyl]-5-methoxymethyl -oxazolidin-2-one;
(RS)-3-[4-(cis- or trans-4-hydroxy-4-methyl-cyclohexyl)-phenyl]-5-methoxymethyl -oxazolidin-2-one.
(RS)-[trans-4-[4-(5-methoxymethyl-2-oxo-oxazolidin-3-yl)-phenyl]-cyclohexyl]-acetonitrile;
(R)-[trans-4-[4-(5-methoxymethyl-2-oxo-oxazolidin-3-yl)-phenyl]-cyclohexyl]-acetonitrile;
(RS)-4-[4-(5-methoxymethyl-2-oxo-oxazolidin-3-yl)-phenyl]-1-(1-oxo-3-phenyl-2(E) -propenyl)-piperidine;
(RS)-3-(trans-4-[4-(5-methoxymethyl-2-oxo-oxazolidin-3-yl)-phenyl]-cyclohexyloxy]-ethanamine;
(R)-[trans-4-[4-(5-methoxymethyl-2-oxo-oxazolidin-3-yl)-phenyl]-cyclohexyloxy]-acetonitrile;
(RS)-trans-4-[4-(5-methoxymethyl-2-oxo-oxazolidin-3-yl)-phenyl]-cyclohexyloxy]-acetonitrile; and,
(R)-3-[4-[trans-4-(3-amino-propoxy)-cyclohexyl]-phenyl]-5-methoxymethyl-oxazolidin-2-one;
   or pharmaceutically acceptable salts thereof.

[33] In another embodiment, the MAO-B inhibitor is selected from the group of compounds described U.S. Pat. No. 5,843,975 or pharmaceutically acceptable salts thereof. Compounds from this publication include compounds of formula XVII or stereoisomers or pharmaceutically acceptable salts thereof:

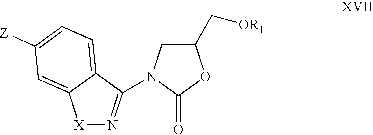

wherein:

X is selected from O, S, and NR;

R₁ is selected from H and C₁₋₄ alkyl;

Z is selected from H, Me, OR₃, —CH═CH—R_{4,} and —CH₂CH₂R₄;

R₃ is selected from H and a benzyl group, which is optionally substituted by a group selected from halogen, NO₂, —OCH₂O—, CH₃OC₂CH₂—, butyl, 4,4,4-trifluorobutyl, 4,4,4-trifluoro-3-hydroxybutyl, and 4,4,4-trifluorobut-2-enyl group; and,

R₄ is selected from phenyl, 3,3,3-trifluoropropyl, and 3,3,3-trifluoro-2-hydroxypropyl.

[34] Examples of compounds of formula XVII include:

(S)-5-Methoxymethyl-3->6-(4,4,4-trifluorobutoxy)-1,2-benzisoxazol-3-yl!oxazolidin-2-one or a pharmaceutically acceptable salt thereof.

[35] In another embodiment, the MAO-B inhibitor is selected from the group of compounds described U.S. Pat. No. 5,965,591 or pharmaceutically acceptable salts thereof. Compounds from this publication include compounds of formula XVIII or pharmaceutically acceptable salts thereof:

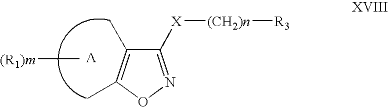

wherein:

R₁ is selected from H; halogen; C₁₋₆ alkyl; C₁₋₄ alkyl substituted by a halogen atom or a C₁₋₄ alkoxy; C₁₋₆ alkoxy; halogeno-C₁₋₆ alkoxy; OH; C₁₋₆ alkylthio; NH₂; C₁₋₆—NH; (C₁₋₆ alkyl)₂N; C₁₋₆ alkanoyl; C₁₋₆ alkanoyl-NH; C₁₋₆ alkanoyloxy; C₁₋₆ alkoxy-carbonyl; CO₂H; (C₁₋₆ alkylthio)thiocarbonyl; carbamoyl; mono-C₁₋₆ alkylcarbamoyl; di-C₁₋₆ alkylcarbamoyl; NO₂; and, —CN;

R₃ is NH₂;

m is 1;

n is selected from 1, 2, 3, 4, 5, and 6;

Ring A is a phenyl ring fused with the isoxazole ring or a naphthyl ring fused with the isoxazole ring; and,

X is selected from O and S.

[36] Examples of compounds of formula XVIII include:
3-(2-aminoethoxy)-1,2-benzisoxazole,
3-(2-aminoethoxy)-5-fluoro-1,2-benzisoxazole,
3-(2-aminoethylthio)-5-fluoro-1,2-benzisoxazole,
3-(2-aminoethoxy)-5-chloro-1,2-benzisoxazole,
3-(2-aminoethylthio)-5-chloro-1,2-benzisoxazole,
3-(2-aminoethoxy)-6-chloro-1,2-benzisoxazole,
3-(2-aminoethoxy)-7-chloro-1,2-benzisoxazole,
3-(2-aminoethoxy)-5-bromo-1,2-benzisoxazole,
3-(2-aminoethoxy)-5-methyl-1,2-benzisoxazole,
3-(2-aminoethoxy)-5-methyl-1,2-benzisoxazole,
3-(2-aminoethoxy)-6-methoxy-1,2-benzisoxazole,
3-(2-aminoethoxy)-7-methyl-1,2-benzisoxazole,
3-(2-aminoethoxy)-5-methoxy-1,2-benzisoxazole,
3-(2-aminoethylthio)-5-methoxy-1,2-benzisoxazole,
3-(2-aminoethoxy)-5-difluoromethoxy-1,2-benzisoxazole,
3-(2-aminoethoxy)-5-methoxycarbonyl-1,2-benzisoxazole,
3-(2-aminoethoxy)-5-nitro-1,2-benzisoxazole,
3-(2-aminoethylthio)-5-nitro-1,2-benzisoxazole,
3-(2-aminoethoxy)-4-cyano-1,2-benzisoxazole,
3-(2-aminoethoxy)-4-carbamoyl-1,2-benzisoxazole hydrochloride,
3-(2-aminoethylthio)-5-amino-1,2-benzisoxazole dihydrochloride,
3-(2-aminoethoxy)-1,2-naphtho[2,3-e]isoxazole hydrochloride,
3-(2-aminoethoxy)-5-methylamino-1,2-benzisoxazole dihydrochloride,
3-(2-aminoethoxy)-5-dimethylamino-1,2-benzisoxazole dihydrochloride,
3-(2-aminoethoxy)-7-carboxy-1,2-benzisoxazole hydrochloride,
3-(2-aminoethoxy)-5-hydroxy-1,2-benzisoxazole hydrochloride, and
3-(2-aminoethoxy)-5-acetoxy-1,2-benzisoxazole hydrochloride,
   or pharmaceutically acceptable salts thereof.

[37] In another embodiment, the MAO-B inhibitor is selected from the group of compounds described U.S. Pat. No. 6,005,116 or pharmaceutically acceptable salts thereof. Compounds from this publication include compounds of formula XIX or pharmaceutically acceptable salts thereof:

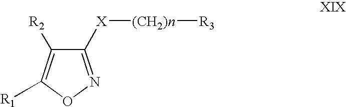

wherein:

X is selected from O and S;

R₁ is selected from C₆₋₁₄aryl substituted with 0-3 substituents or a 5-6-membered aromatic heterocyclic group substituted with 0-3 substituents and consisting of carbon atoms and 1-2 heteroatoms independently selected from nitrogen, oxygen and sulfur atoms;

the substituents for R₁ are independently selected from halogen; C₁₋₆ alkyl; C₁₋₆ alkyl substituted with a halogen or a C₁₋₆ alkoxy; C₁₋₆ alkoxy; C₆₋₁₄ aryl, C₇₋₁₈ aralkyl; C₆₋₁₄ aryloxy; and C₇₋₁₈ aralkyloxy, wherein the aralkyloxy group is substituted with 0-3 substituents independently selected from halogen; C₁₋₆ alkyl; C₁₋₆ alkoxy; —CN; NO₂; OH; C₁₋₇ alkanoyl; C₁₋₇ alkanoyloxy; C₂₋₇ alkoxycarbonyl; NH₂; a carbamoyl; a mono(C₁₋₆ alkyl)carbamoyl; a di(C₁₋₆ alkyl)carbamoyl, and a mono C₇₋₁₅ arylcarbonylamino substituted with 0-3 substituents selected from a halogen, C₁₋₆ alkyl, and C₁₋₆ alkoxy;

R₂ is selected from H; halogen; C₁₋₆ alkyl substituted with a halogen or C₁₋₆ alkoxy; C₂₋₆ alkenyl; C₂₋₆ alkynyl; C₃₋₁₀ cycloalkyl; C₃₋₁₀ cycloalkenyl; C₁₋₆ CO₂H; C₁₋₇ alkanoyl; C₂₋₇ alkoxycarbonyl; carbamoyl; mono-C₁₋₆ alkyl-carbamoyl; and, di-C₁₋₆ alkyl-carbamoyl;

R₃ is selected from NH₂; C₁₋₆ alkyl-NH; (C₁₋₆ alkyl)₂N; C₁₋₇ alkanoylNH; C₂₋₇ alkoxycarbonyl-NH; C₇₋₁₅ arylcarbonyl-NH-substituted with 0-3 substituents independently selected from halogen, C₁₋₆ alkyl, and C₁₋₆ alkoxy; and a 5-6-membered saturated heterocyclic group (attached through a ring nitrogen atom), which consists of carbon atoms, one nitrogen atom, and an additional nitrogen or oxygen atom; and,

n is selected from 2, 3, 4, 5, and 6.

[38] Examples of compounds of formula XIX include:
3-(2-aminoethoxy)-5-phenylisoxazole,
3-(2-aminoethoxy)-4-chloro-5-phenylisoxazole,
3-(2-aminoethoxy)-4-ethyl-5-phenylisoxazole,
3-(2-aminoethoxy)-5-phenyl-4-propylisoxazole,
3-(2-aminoethoxy)-4-isopropyl-5-phenylisoxazole,
3-(2-aminoethoxy)-4-isobutyl-5-phenylisoxazole,
3-(2-aminoethoxy)-5-(2-chlorophenyl)-4-isopropylisoxazole,
3-(2-aminoethoxy)-5-(4-chlorophenyl)isoxazole,
3-(2-aminoethoxy)-5-(4-chlorophenyl)-4-isopropylisoxazole,
3-(2-aminoethoxy)-5-(2,4-dichlorophenyl)-4-isopropylisoxazole,
3-(2-aminoethoxy)-5-(2-furyl)-4-isopropylisoxazole,
3-(2-aminoethoxy)-5-(2-thienyl)isoxazole,
3-(2-aminoethoxy)-4-chloro-5-(2-thienyl)isoxazole,
3-(2-aminoethoxy)-4-isopropyl-5-(2-thienyl)isoxazole, and
4-allyl-3-(2-aminoethoxy)-5-phenylisoxazole,
   and pharmaceutically acceptable salts thereof.

[39] In another embodiment, the MAO-B inhibitor is selected from the group of compounds described U.S. Pat. No. 6,660,736 or pharmaceutically acceptable salts thereof. Compounds from this publication include compounds of formula XX or pharmaceutically acceptable salts thereof:

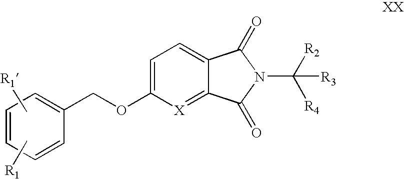

wherein:

X is selected from N and CH;

R₁ and R₁′ are independently selected from H, halogen, C₁₋₆ alkyl, halo C_{1 6} alkyl, —CN, C₁₋₆ alkoxy, C₁₋₆ haloalkoxy, and CF₃;

R₂ is selected from H, (CH₂)ₙCN, (CH₂)ₚOR₄, (CH₂)ₙCON(R₄)₂, (CH₂)ₙCO₂R₄, CHR₅(CH₂)ₙOR_{6,} (CH₂)ₙN(R₄)₂, (CH₂)ₙNHCOR₄, and (CH₂)ₙNHCOOR₄;

R₃ is selected from H, alkyl, (CH₂)ₙO—C₁₋₆ alkyl, (CH₂)ₙS—C₁₋₆ alkyl, (CH₂)ₙS(O)—C₁₋₆ alkyl, benzyl, and —CN;

R₄ is independently selected from H and alkyl;

R₅ is selected from H, C₁₋₆ alkyl, —CN, and CONH₂; and,

R₆ is selected from H and C₁₋₆ alkyl.

[40] Examples of compounds of formula XX include:
2-[5-(4-fluoro-benzyloxy)-1,3-dioxo-1,3-dihydro-isoindol-2-yl]-acetamide,
(S)-2-[5-(4-fluoro-benzyloxy)-1,3-dioxo-1,3-dihydro-isoindol-2-yl]-propionamide,
(S)-2-[5-(4-fluoro-benzyloxy)-1,3-dioxo-1,3-dihydro-isoindol-2-yl]-3-hydroxy-propionamide,
(R)-2-[5-(4-fluoro-benzyloxy)-1,3-dioxo-1,3-dihydro-isoindol-2-yl]-propionamide,
2-[5-(3-fluoro-benzyloxy)-1,3-dioxo-1,3-dihydro-isoindol-2-yl]-propionamide,
2-[5-(3-fluoro-benzyloxy)-1,3-dioxo-1,3-dihydro-isoindol-2-yl]-acetamide,
2-[5-(3-fluoro-benzyloxy)-1,3-dioxo-1,3-dihydro-isoindol-2-yl]-3-hydroxy-propionamide,
N-{2-[5-(4-fluoro-benzyloxy)-1,3-dioxo-1,3-dihydro-isoindol-2-yl]-ethyl}-acetamide,
2-(2-amino-ethyl)-5-(4-fluoro-benzyloxy)-isoindole-1,3-dione,
5-(4-fluoro-benzyloxy)-2-piperidin-4-yl-isoindole-1,3-dione,
5-(4-fluoro-benzyloxy)-2-(2-hydroxy-ethyl)-isoindole-1,3-dione,
5-(4-fluoro-benzyloxy)-2-(2-methoxy-ethyl)-isoindole-1,3-dione,
5-(3-fluoro-benzyloxy)-2-(2-methoxy-ethyl)-isoindole-1,3-dione,
(S)-5-(4-fluoro-benzyloxy)-2-(2-methoxy-1-methyl-ethyl)-isoindole-1,3-dione,
(S)-5-(3-fluoro-benzyloxy)-2-(2-methoxy-1-methyl-ethyl)-isoindole-1,3-dione,
(S)-5-(2-fluoro-benzyloxy)-2-(2-methoxy-1-methyl-ethyl)-isoindole-1,3-dione,
(S)-2-(2-methoxy-1-methyl-ethyl)-5-(4-trifluoromethyl-benzyloxy)-isoindole-1,3-dione,
(S)-5-(4-bromo-benzyloxy)-2-(2-methoxy-1-methyl-ethyl)-isoindole-1,3-dione,
(S)-5-(3,4-difluoro-benzyloxy)-2-(2-methoxy-1-methyl-ethyl)-isoindole-1,3-dione,
5-(3-fluoro-benzyloxy)-2-(2-hydroxy-ethyl)-isoindole-1,3-dione,
5-(4-fluoro-benzyloxy)-2-(3,3,3-trifluoro-2-hydroxy-propyl)-isoindole-1,3-dione,
5-(3,5-bis-trifluoromethyl-benzyloxy)-2-(2-methoxy-1-methyl-ethyl)-isoindole-1,3-dione,
2-(2-ethylsulfanyl-ethyl)-5-(4-fluoro-benzyloxy)-isoindole-1,3-dione,
(S)-2-[5-(4-fluoro-benzyloxy)-1,3-dioxo-1,3-dihydro-isoindol-2-yl]-thiopropionamide,
2-(2-ethylsulfanyl-ethyl)-5-(3-fluoro-benzyloxy)-isoindole-1,3-dione,
5-(4-fluoro-benzyloxy)-1,3-dioxo-1,3-dihydro-isoindol-2-yl]-acetonitrile, and
[5-(3-fluoro-benzyloxy)-1,3-dioxo-1,3-dihydro-isoindol-2-yl]-acetonitrile,
   or pharmaceutically acceptable salts thereof.

[41] In another embodiment, the MAO-B inhibitor is selected from the group of compounds described U.S. Pat. No. 6,667,327 or pharmaceutically acceptable salts thereof. Compounds from this publication include compounds of formula XXI or pharmaceutically acceptable salts thereof:

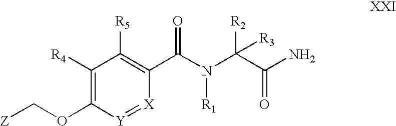

wherein:

X and Y are independently selected from N and CR₆;

Z is selected from C₁₋₆-haloalkyl, aryl, aryl substituted by one or more substituents selected from C₁₋₆ alkyl, halogen, C₁₋₆ haloalkyl, C₁₋₆ alkoxy, and —CN;

R₁ is selected from H and C₁₋₆ alkyl;

R₂ is selected from H and C₁₋₆ alkyl;

R₃ is selected from H and C₁₋₆ alkyl;

R₄ is selected from H and C₁₋₆ alkyl;

R₅ is selected from H and C₁₋₆ alkyl; and,

R₆ is selected from H and C₁₋₆ alkyl.

[42] Examples of compounds of formula XXI include:
5-(3-fluoro-benzyloxy)-pyridine-2-carboxylic acid carbamoylmethyl-amide,
5-(4-fluoro-benzyloxy)-pyridine-2-carboxylic acid carbamoylmethyl-amide,
5-(3,4-difluoro-benzyloxy)-pyridine-2-carboxylic acid carbamoylmethyl-amide,
(S)-5-(3-fluoro-benzyloxy)-pyridine-2-carboxylic acid (1-carbamoyl-ethyl)-amide,
(S)-5-(4-fluoro-benzyloxy)-pyridine-2-carboxylic acid (1-carbamoyl-ethyl)-amide,
(S)-5-(3,4-difluoro-benzyloxy)-pyridine-2-carboxylic acid (1-carbamoyl-ethyl)-amide,
6-Benzyloxy-N-carbamoylmethyl-nicotinamide,
N-Carbamoylmethyl-6-(3-fluoro-benzyloxy)-nicotinamide,
N-Carbamoylmethyl-6-(4-fluoro-benzyloxy)-nicotinamide,
(S)-6-Benzyloxy-N-(1-carbamoyl-ethyl)-nicotinamide,
(S)-N-(1-Carbamoyl-ethyl)-6-(3-fluoro-benzyloxy)-nicotinamide, and
(S)-N-(1-Carbamoyl-ethyl)-6-(4-fluoro-benzyloxy)-nicotinamide,
   or pharmaceutically acceptable salts thereof.

[43] In another embodiment, the MAO-B inhibitor is selected from the group of compounds described U.S. Pat. No. 6,762,320 or pharmaceutically acceptable salts thereof. Compounds from this publication include compounds of formula XXII or pharmaceutically acceptable salts thereof:

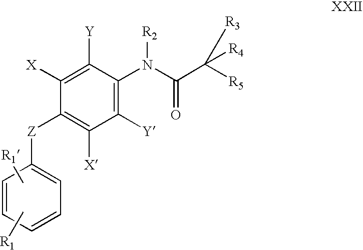

wherein:

R₁ and R₁′ are independently selected from H, halogen, C₁₋₆ alkyl, C₁₋₆ haloalkyl, —CN, C₁₋₆ alkoxy, C₁₋₆ haloalkoxy, CF₃, OH, and CHO;

X and Y are independently selected from H, halogen, C₁₋₆ alkyl, C₁₋₆ haloalkyl, —CN, C₁₋₆ alkoxy, C₁₋₆ haloalkoxy, and CF₃;

X′ and Y′ are independently selected from H, halogen, C₁₋₆ alkyl, C₁₋₆ haloalkyl, —CN, C₁₋₆ alkoxy, C₁₋₆ haloalkoxy, and CF₃;

R₂ is selected from H and C₁₋₆ alkyl;

R₃ and R₄ are independently selected from H, C₁₋₆ alkyl, C₁₋₆ alkoxy, and —CO₂—C₁₋₆ alkyl;

alternatively, R₃ and R_{4,} together with the C-atom to which they are attached, form a C₁₋₇-cycloalkyl ring;

R₅ is selected from CONR₆R₇, CO₂—C₁₋₆ alkyl, —CN, N(R)₂, and NHC(O)R;

R₆ and R₇ are independently selected from H, C₁₋₆ alkyl, NH₂, and OH;

R is H or C₁₋₆ alkyl;

Z is selected from —CHRO—, —OCHR—, —CH₂S—, —SCH₂—, —CH₂CH₂—, —CH═CH—, and —C═C—; and,

n is selected from 0, 1, 2, and 3.

[44] Examples of compounds of formula XXII include:
N-[4-(3-fluoro-benzyloxy)-phenyl]-malonamic acid methyl ester;
N-[3-fluoro-4-(3-fluoro-benzyloxy)-phenyl]-malonamic acid methyl ester;
N-[4-(4-fluoro-benzyloxy)-phenyl]-malonamic acid methyl ester;
N-[2-fluoro-4-(3-fluoro-benzyloxy)-phenyl]-malonamic acid methyl ester;
N-[4-(2,4-difluoro-benzyloxy)-phenyl]-malonamic acid methyl ester;
N-[4-(2-fluoro-benzyloxy)-phenyl]-malonamic acid methyl ester;
N-[4-(2,4,5-trifluoro-benzyloxy)-phenyl]-malonamic acid methyl ester;
N-[2-fluoro-4-(4-fluoro-benzyloxy)-phenyl]-malonamic acid methyl ester;
N-[4-(3,5-bis-trifluoromethyl-benzyloxy)-2-fluoro-phenyl]-malonamic acid methyl ester;
N-[4-(3-fluoro-benzyloxy)-3-methyl-phenyl]-malonamic acid methyl ester;
N-[3-chloro-4-(3-fluoro-benzyloxy)-phenyl]-malonamic acid methyl ester;
cyclopropane-1,1-dicarboxylic acid amide [4-(3-fluoro-benzyloxy)-phenyl]-amide;
N-[4-(3-fluoro-benzyloxy)-phenyl]-malonamide;
N-[4-(3-fluoro-benzyloxy)-phenyl]-2-methyl-malonamide;
N-[3-fluoro-4-(3-fluoro-benzyloxy)-phenyl]-malonamide;
N-[4-(4-fluoro-benzyloxy)-phenyl]-malonamide;
N-[4-(2,4-difluoro-benzyloxy)-phenyl]-malonamide;
N-[4-(2,4,5-trifluoro-benzyloxy)-phenyl]-malonamide;
N-[4-(2-fluoro-benzyloxy)-phenyl]-malonamide;
N-(4-benzyloxy-phenyl)-malonamide;
N-[4-(4-chloro-benzyloxy)-phenyl]-malonamide;
N-[4-(3-fluoro-benzyloxy)-2-hydroxy-phenyl]-malonamide;
N-[2-fluoro-4-(4-fluoro-benzyloxy)-phenyl]-malonamide;
N-[4-(3-fluoro-benzyloxy)-3 -methyl-phenyl]-malonamide;
N-[3-chloro-4-(3-fluoro-benzyloxy)-phenyl]-malonamide;
cyclopropane-1,1-dicarboxylic acid amide [2-fluoro-4-(4-fluoro-benzyloxy)-phenyl]-amide;
2-Acetylamino-N-[2-fluoro-4-(4-fluoro-benzyloxy)-phenyl]-acetamide;
2-Acetylamino-N-[2-fluoro-4-(3-fluoro-benzyloxy)-phenyl]-acetamide;
N-[2-Fluoro-4-(4-fluoro-benzyloxy)-phenyl]-2-formylamino-acetamide;
N-[2-Fluoro-4-(3-fluoro-benzyloxy)-phenyl]-2-formylamino-acetamide;
11) 2-amino-N-[2-fluoro-4-(4-fluoro-benzyloxy)-phenyl]-acetamide;
14) N-{4-[2-(4-fluoro-phenyl)-vinyl]-phenyl}-malonamic acid methyl ester;
N-{4-[2-(3-fluoro-phenyl)-vinyl]-phenyl}-malonamide;
N-{4-[2-(4-fluoro-phenyl)-vinyl]-phenyl}-malonamide;
N-{4-[2-(3-fluoro-phenyl)-vinyl]-phenyl}-malonamic acid methyl ester.
N-[4-(3-Fluoro-benzyloxy)-phenyl]-2-methyl-malonamic acid methyl ester;
N-[4-(3-Fluoro-benzyloxy)-phenyl]-2-methoxy-malonamic acid methyl ester;
N-[4-(3-Fluoro-benzyloxy)-2-trifluoromethyl-phenyl]-malonamic acid methyl ester;
N-[4-(3-Fluoro-benzyloxy)-phenyl]-N-methyl-malonamic acid methyl ester;
N-[4-(4-Trifluoromethyl-benzyloxy)-phenyl]-malonamic acid methyl ester;
N-(4-Benzyloxy-phenyl)-malonamic acid methyl ester;
N-[2-Fluoro-4-(4-trifluoromethyl-benzyloxy)-phenyl]-malonamic acid methyl ester;
N-[2-Fluoro-4-(4-fluoro-benzyloxy)-phenyl]-malonamic acid ethyl ester;
N-[4-(3-Fluoro-benzyloxy)-3-formyl-phenyl]-malonamic acid methyl ester;
N-[4-(3-Fluoro-benzyloxy)-3-methoxy-phenyl]-malonamic acid methyl ester; and
N-[2-Fluoro-4-(4-fluoro-benzyloxy)-phenyl]-2,2-dimethyl-malonamic acid methyl ester.
N-[4-(3-Fluoro-phenoxymethyl)-phenyl]-malonamic acid methyl ester;
N-[4-(3-Fluoro-benzylsulfanyl)-phenyl]-malonamic acid methyl ester;
2-[4-(3-Fluoro-benzyloxy)-phenylcarbamoyl]-malonic acid dimethyl ester;
N-{4-[2-(4-Fluoro-phenyl)-vinyl]-phenyl}-malonamic acid methyl ester;
N-{4-[2-(3-Fluoro-phenyl)-vinyl]-phenyl}-malonamic acid methyl ester;
N-{4-[2-(4-Fluoro-phenyl)-ethyl]-phenyl}-malonamic acid methyl ester;
N-{4-[2-(3-Fluoro-phenyl)-ethyl]-phenyl}-malonamic acid methyl ester;
N-{4-[2-(4-Methoxy-phenyl)-vinyl]-phenyl}-malonamic acid methyl ester; N-{4-[2-(4-Chloro-phenyl)-ethyl]-phenyl}-malonamic acid methyl ester.
N-[4-(3-Fluoro-benzyloxy)-phenyl]-2,2-dimethyl-malonamide;
N-[4-(4-Trifluoromethyl-benzyloxy)-phenyl]-malonamide;
N-[2-Fluoro-4-(3-fluoro-benzyloxy)-phenyl]-malonamide;
N-[2,5-Difluoro-4-(3-fluoro-benzyloxy)-phenyl]-malonamide;
N-[4-(3-Fluoro-benzyloxy)-phenyl]-N′-hydroxy-malonamide;
N-[4-(3,5 -Bis-trifluoromethyl-benzyloxy)-2-fluoro-phenyl]-malonamide;
N-[2-Fluoro-4-(4-trifluoromethyl-benzyloxy)-phenyl]-malonamide;
N-[4-(3-Fluoro-benzyloxy)-3 -methoxy-phenyl]-malonamide;
N-[4-(3-Fluoro-benzylsulfanyl)-phenyl]-malonamide;
N-{4-[1-(3-Fluoro-phenyl)-ethoxy]-phenyl}-malonamide;
N-[4-(3-Fluoro-phenoxymethyl)-phenyl]-malonamide;
2-Ethyl-N-[4-(3 -fluoro-benzyloxy)-phenyl]-malonamide;
N-{4-[2-(4-Fluoro-phenyl)-vinyl]-phenyl}-malonamide;
N-{4-[2-(3-Fluoro-phenyl)-vinyl]-phenyl}-malonamide;
N-{4-[2-(4-Fluoro-phenyl)-ethyl]-phenyl}-malonamide;
N-{4-[2-(4-Chloro-phenyl)-ethyl]-phenyl}-malonamide.
2-Cyano-N-[4-(3-fluoro-benzyloxy)-phenyl]-acetamide;
N-[4-(3-Fluoro-benzyloxy)-phenyl]-2-hydrazinocarbonyl-acetamide;
Cyclopropane-1,1-dicarboxylic acid amide [4-(3-fluoro-phenoxymethyl)-phenyl]-amide;
2-Amino-N-[2-fluoro-4-(4-fluoro-benzyloxy)-phenyl]-acetamide (1:1) hydrochloride;
(R)-2-Amino-N-[2-fluoro-4-(4-fluoro-benzyloxy)-phenyl]-propionamide;
2-Amino-N-[2-fluoro-4-(4-fluoro-benzyloxy)-phenyl]-propionamide;
1-[2-Fluoro-4-(4-fluoro-benzyloxy)-phenylcarbamoyl]-2S-methyl-propyl-ammonium chloride;
(R)-2-Acetylamino-N-[2-fluoro-4-(4-fluoro-benzyloxy)-phenyl]-propionamide;
(R)-N-[2-Fluoro-4-(4-fluoro-benzyloxy)-phenyl]-2-formylamino-propionamide;
2-Amino-N-[2-fluoro-4-(3-fluoro-benzyloxy)-phenyl]-acetamide hydrochloride (1:1);
2-Amino-N-[2-fluoro-4-(4-trifluoromethyl-benzyloxy)-phenyl]-acetamide (1:1) hydrochloride;
2-Amino-N-[4-(3,5-bis-trifluoromethyl-benzyloxy)-2-fluoro-phenyl]-acetamide (1:1) hydrochloride;
2-Acetylamino-N-[4-(3,5-bis-trifluoromethyl-benzyloxy)-2-fluoro-phenyl]-acetamide; and,
2-Amino-N-[4-(3-fluoro-benzyloxy)-phenyl]-acetamide hydrochloride;
   or pharmaceutically acceptable salts thereof.

[45] In another embodiment, the MAO-B inhibitor is selected from the group of compounds described in U.S. Pat. No. 6,846,818 or pharmaceutically acceptable salts thereof. Compounds from this publication include compounds of formulas XXIII, XXIV, and XXV, or pharmaceutically acceptable salts thereof:

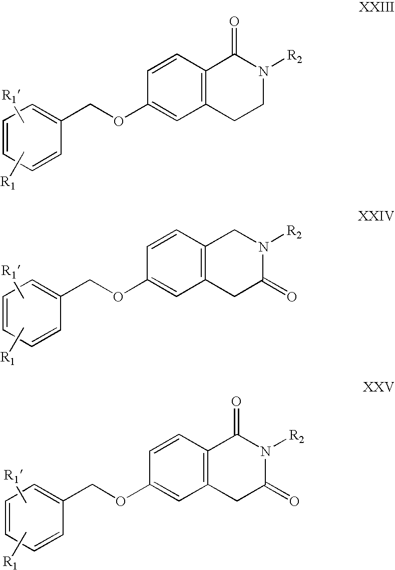

wherein:

R₁ is selected from H, halogen, C₁₋₆ alkyl, C₁₋₆ haloalkyl, —CN, C₁₋₆ alkoxy, C₁₋₆ haloalkoxy, and CF₃;

R₁′ is selected from H, halogen, C₁₋₆ alkyl, C₁₋₆ haloalkyl, —CN, C₁₋₆ alkoxy, C₁₋₆ haloalkoxy, and CF₃;

R₂ is selected from H and CR₃R₄R₅;

R₃ is selected from (CH₂)ₙCO—NR₆R₇, (CH₂)ₙCN, (CH₂)ₚOR₈, (CH₂)ₙNR₆R₇, (CH₂)ₙNHCOR₉, (CH₂)ₚSR_{8,} and (CH₂)ₚSOR₉;

R₄ is selected from H, C₁₋₆ alkyl, (CH₂)ₚOR₈, (CH₂)ₚSR₈, and benzyl;

R₅ is selected from H, C₁₋₆ alkyl, (CH₂)ₚOR₈, (CH₂)ₚSR₈, and benzyl;

R₆ and R₇ are independently selected from H and C₁₋₆ alkyl;

R₈ is selected from H and C₁₋₆ alkyl;

R₉ is C₁₋₆ alkyl;

n is selected from 0, 1, and 2; and,

p is selected from 1 and 2.

[46] Examples of compounds of formulas XXIII, XXIV, and XXV include:
2-[6-(3-fluoro-benzyloxy)-1-oxo-3,4-dihydro-1H-isoquinolin-2-yl]-acetamide,
2-[6-(3-fluoro-benzyloxy)-1-oxo-3,4-dihydro-1H-isoquinolin-2-yl]-propionamide
2-[6-(4-fluoro-benzyloxy)-1-oxo-3,4-dihydro-1H-isoquinolin-2-yl]-propionamide,
2-[6-(3,4-difluoro-benzyloxy)-1-oxo-3,4-dihydro-1H-isoquinolin-2-yl]-propionamide,
2-[6-(3-fluoro-benzyloxy)-1-oxo-3,4-dihydro-1H-isoquinolin-2-yl]-propionamide;
2-(R)-[6-(3-fluoro-benzyloxy)-1-oxo-3,4-dihydro-1H-isoquinolin-2-yl]-propionamide,
2-(R)-[6-(4-fluoro-benzyloxy)-1-oxo-3,4-dihydro-1H-isoquinolin-2-yl]-propionamide,
2-(S)-[6-(4-fluoro-benzyloxy)-1-oxo-3,4-dihydro-1H-isoquinolin-2-yl]-propionamide,
2-(S)-[6-(4-fluoro-benzyloxy)-1-oxo-3,4-dihydro-1H-isoquinolin-2-yl]-3-hydroxy-propionamide,
2-(R)-[6-(2,6-difluoro-benzyloxy)-1-oxo-3,4-dihydro-1H-isoquinolin-2-yl]-propionamide;
2-[6-(3-fluoro-benzyloxy)3,4-dihydro-1H-isoquinolin-2-yl]-propionamide,
2-[6-(4-fluoro-benzyloxy)3,4-dihydro-1H-isoquinolin-2-yl]-acetamide,
2-[6-(3-fluoro-benzyloxy)-3,4-dihydro-1H-isoquinolin-2-yl]-acetamide,
2-[6-(4-fluoro-benzyloxy)3,4-dihydro-1H-isoquinolin-2-yl]-propionamide;
2-(R)-[6-(4-fluoro-benzyloxy)-1,3-dioxo-3,4-dihydro-1H-isoquinolin-2-yl]-propionamide,
2-(S)-[6-(4-fluoro-benzyloxy)-1,3-dioxo-3,4-dihydro-1H-isoquinolin-2-yl]-propionamide;
2-(S)-[6 (4-fluoro-benzyloxy)-3-oxo-3,4-dihydro-1H-isoquinolin-2-yl]-propionamide,
2(R)-[6-(4-fluoro-benzyloxy)-3-oxo-3,4-dihydro-1H-isoquinolin-2-yl]-propionamide
2-[1-oxo-6-(4-trifluoromethyl-benzyloxy)-3,4-dihydro-1H-isoquinolin-2-yl]-propionamide;
2-[6-(2-Fluoro-benzyloxy)-1-oxo-3,4-dihydro-1H-isoquinolin-2-yl]-propionamide;
2(R)-[6-(2,6-Difluoro-benzyloxy)-1-oxo-3,4-dihydro-1H-isoquinolin-2-yl]-propionamide;
2(R)-[6-(2-Fluoro-benzyloxy)-1-oxo-3,4-dihydro-1H-isoquinolin-2-yl]-propionamide;
2(R)[6-(2,3-Difluoro-benzyloxy)-1-oxo-3,4-dihydro-1H-isoquinolin-2-yl]-propionamide;
2(R)[6-(3-Fluoro-benzyloxy)-1-oxo-3,4-dihydro-1H-isoquinolin-2-yl]-propionamide;
2(S)-[6-(3-Fluoro-benzyloxy)-1-oxo-3,4-dihydro-1H-isoquinolin-2-yl]-propionamide; and
2(S)-[6-(3,4-Difluoro-benzyloxy)-1-oxo-3,4-dihydro-1H-isoquinolin-2-yl]-propionamide;
6-(3-Fluoro-benzyloxy)-3,4-dihydro-2H-isoquinolin-1-one;
2-[6-(4-Fluoro-benzyloxy)-1-oxo-3,4-dihydro-1H-isoquinolin-2-yl]-acetamide;
2(R)-[6-(3-Cyano-benzyloxy)-1-oxo-3,4-dihydro-1H-isoquinolin-2-yl]-propionamide
2-[1-oxo-6-(4-trifluoromethyl-benzyloxy)-3,4-dihydro-1H-isoquinolin-2-yl]-propionamide;
2-[6-(2-Fluoro-benzyloxy)-1-oxo-3,4-dihydro-1H-isoquinolin-2-yl]-propionamide;
2(R)-[6-(2,6-Difluoro-benzyloxy)-1-oxo-3,4-dihydro-1H-isoquinolin-2-yl]-propionamide;
2(R)-[6-(2-Fluoro-benzyloxy)-1-oxo-3,4-dihydro-1H-isoquinolin-2-yl]-propionamide;
2(R)[6-(2,3-Difluoro-benzyloxy)-1-oxo-3,4-dihydro-1H-isoquinolin-2-yl]-propionamide;
2(R)[6-(3-Fluoro-benzyloxy)-1-oxo-3,4-dihydro-1H-isoquinolin-2-yl]-propionamide;
2(S)-[6-(3-Fluoro-benzyloxy)-1-oxo-3,4-dihydro-1H-isoquinolin-2-yl]-propionamide;
2(S)-[6-(3,4-Difluoro-benzyloxy)-1-oxo-3,4-dihydro-1H-isoquinolin-2-yl]-propionamide;
6-(3-Fluoro-benzyloxy)-3,4-dihydro-2H-isoquinolin-1-one;
2-[6-(4-Fluoro-benzyloxy)-1-oxo-3,4-dihydro-1H-isoquinolin-2-yl]-acetamide;
2(R)-[6-(3-Cyano-benzyloxy)-1-oxo-3,4-dihydro-1H-isoquinolin-2-yl]-propionamide
2(R)-[6-(3,5-Difluoro-benzyloxy)-1-oxo-3,4-dihydro-1H-isoquinolin-2-yl]-propionamide;
3-[6-(4-Fluoro-benzyloxy)-3,4-dihydro-1H-isoquinolin-2-yl]-propionamide;
3-[6-(3-Fluoro-benzyloxy)-3,4-dihydro-1H-isoquinolin-2-yl]-propionamide;
2(R)-[(3,4-Difluoro-benzyloxy)-1-oxo-3,4-dihydro-1H-isoquinolin-2-yl]-propionamide;
2-[6-(3-Chloro-benzyloxy)-1-oxo-3,4-dihydro-1H-isoquinolin-2-yl]-propionamide;
3-[6-(3-Fluoro-benzyloxy)-1-oxo-3,4-dihydro-1-isoquinolin-2-yl]-propionamide;
2-(S)-[6-(4-Fluoro-benzyloxy)-1-oxo-3,4-dihydro-1H-isoquinolin-2-yl]-4-methylsulfanyl-butyramide;
2-(R)-[6-(4-Fluoro-benzyloxy)-1-oxo-3,4-dihydro-1H-isoquinolin-2-yl]-3-hydroxy-propionamide;
2-(S)-[6-(4-Fluoro-benzyloxy)-1-oxo-3,4-dihydro-1H-isoquinolin-2-yl]-4-methyl-pentanoic acid amide;
2-(S)-[6-(4-Fluoro-benzyloxy)-1-oxo-3,4-dihydro-1H-isoquinolin-2-yl]-butyramide;
2-(R)-[6-(4-Fluoro-benzyloxy)-1-oxo-3,4-dihydro-H-isoquinolin-2-yl]-3-phenyl1-propionamide;
2-(S)-[6-(4-Fluoro-benzyloxy)-1-oxo-3,4-dihydro-1H-isoquinolin-2-yl]-3-methyl-butyramide;
2-(S)-[6-(4-Fluoro-benzyloxy)-1-oxo-3,4-dihydro-1H-isoquinolin-2-yl]-3-phenyl-propionamide;
2-[6-(4-Fluoro-benzyloxy)-1-oxo-3,4-dihydro-1H-isoquinolin-2-yl]-isobutyramide;
6-(3-Fluoro-benzyloxy)-2-(2-hydroxy-1-methyl-ethyl)-3,4-dihydro-2H-isoquinolin-1-one;
6-(3-Fluoro-benzyloxy)-2-(2-hydroxy-ethyl)-3,4-dihydro-2H-isoquinolin-1-one;
6-(3-Fluoro-benzyloxy)-2-(2-methoxy-1-methyl-ethyl)-3,4-dihydro-2H-isoquinolin-1-one;
6-(3-Fluoro-benzyloxy)-2-(2-methoxy-ethyl)-3,4-dihydro-2H-isoquinolin-1-one;
2-(2-Ethoxy-ethyl)-6-(3-fluoro-benzyloxy)-1,2,3,4-tetrahydro-isoquinoline;
6-(4-Fluoro-benzyloxy)-2-(2-methoxy-ethyl)-1,2,3,4-tetrahydro-isoquinoline;
2-(2-Ethoxy-ethyl)-6-(4-fluoro-benzyloxy)-1,2,3,4-tetrahydro-isoquinoline;
[6-(3-Fluoro-benzyloxy)-1-oxo-3,4-dihydro-1H-isoquinolin-2-yl]-acetonitrile;
3-[6-(3-Fluoro-benzyloxy)-1-oxo-3,4-dihydro-1H-isoquinolin-2-yl]-propionitrile;
6-(4-Fluoro-benzyloxy)-2-(4,4,4-trifluoro-butyl)-1,2,3,4-tetrahydro-isoquinoline;
6-(4-Fluoro-benzyloxy)-2-(tetrahydro-furan-2-ylmethyl)-1,2,3,4-tetrahydro-isoquinoline;
2-[6 (4-Fluoro-benzyloxy)-1-oxo-3,4-dihydro-1H-isoquinolin-2-yl]-propionamide;
2-(R)-[6-(3-Fluoro-benzyloxy)-1-oxy-3,4-dihydro-1H-isoquinolin-2-yl]-propionamide;
2(R)-[6-(4-Fluoro-benzyloxy)-1-oxo-3,4-dihydro-1H-isoquinolin-2-yl]-propionamide; and,
2(S)-[6-(4-Fluoro-benzyloxy)-1-oxo-3,4-dihydro-1H-isoquinolin-2-yl]-propionamide,
   or pharmaceutically acceptable salts thereof.

[47] In another embodiment, the MAO-B inhibitor is selected from the group of compounds described U.S. Pat. No. 6,846,832 or pharmaceutically acceptable salts thereof. Compounds from this publication include compounds of formula XXVI and XXVII, or pharmaceutically acceptable salts thereof:

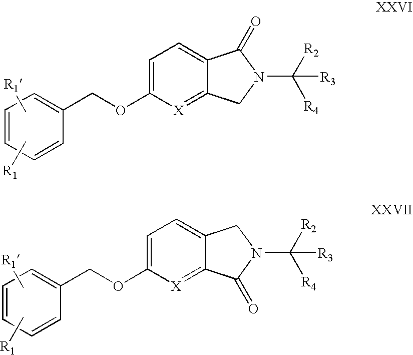

wherein:

X is selected from N and CH;

R₁ and R₁′ are independently selected from H, halogen, C₁₋₆ alkyl, C₁₋₆ haloalkyl, —CN, C₁₋₆ alkoxy, C₁₋₆ haloalkoxy, and CF₃;

R₂ is selected from H, (CH₂)ₙCN, (CH₂)ₙOR₄, (CH₂)ₙCON(R₄)₂, (CH₂)ₙCO₂R₄, CHR₅(CH₂)ₙOR₆, (CH₂)ₙ-isoindole-1,3-dionyl, and (CH₂)ₙN(R₄)₂;

R₃is selected from H, C₁₋₆ alkyl, (CH₂)ₙO—C₁₋₆ alkyl, (CH₂)ₙS—C₁₋₆ alkyl, (CH₂)ₙS(O)—C₁₋₆ alkyl, and benzyl;

R₄ is selected from H and C₁₋₆ alkyl;

R₅ is selected from H, C₁₋₆ alkyl, —CN, and CONH₂;

R₆ is selected from H and C₁₋₆ alkyl; and,

n is selected from 0, 1, and 2.

[48] Examples of compounds of formula XXVI and XXVII include:
2-[5-(3-fluoro-benzyloxy)-1-oxo-1,3-dihydro-isoindol-2-yl]-acetamide,
2-[5-(3-fluoro-benzyloxy)-1-oxo-1,3-dihydro-isoindol-2-yl]-propionamide,
(S)-2-[6-(3-fluoro-benzyloxy)-1-oxo-1,3-dihydro-isoindol-2-yl]-propionamide,
(R)-2-[6-(3-fluoro-benzyloxy)-1-oxo-1,3-dihydro-isoindol-2-yl]-propionamide,
2-[5-(4-fluoro-benzyloxy)-1-oxo-1,3-dihydro-isoindol-2-yl]-acetamide,
2-[1-oxo-5-(4-trifluoromethyl-benzyloxy)-1,3-dihydro-isoindol-2-yl]-acetamide,
5-(3-Fluoro-benzyloxy)-2-(2-methoxy-ethyl)-2,3-dihydro-isoindol-1-one,
2-[6-(3-fluoro-benzyloxy)-1-oxo-1,3-dihydro-isoindol-2-yl]-acetamide,
(R)-2-[6-(3-fluoro-benzyloxy)-1-oxo-1,3-dihydro-isoindol-2-yl]-propionamide,
(S)-2-[1-oxo-6-(4-trifluoromethyl-benzyloxy)-1,3-dihydro-isoindol-2-yl]-propionamide,
(R)-2-[1-oxo-6-(4-trifluoromethyl-benzyloxy)-1,3-dihydro-isoindol-2-yl]-propionamide,
[6-(3-fluoro-benzyloxy)-1-oxo-1,3-dihydro-isoindol-2-yl]-acetic acid methyl ester,
[1-oxo-6-(4-trifluoromethyl-benzyloxy)-1,3-dihydro-isoindol-2-yl]-acetic acid methyl ester,
2-(2-Methoxy-ethyl)-6-(3-fluoro-benzyloxy)-2,3-dihydro-isoindol-1-one,
2-(2-methoxy-ethyl)-6-(4-trifluoromethyl-benzyloxy)-2,3-dihydro-isoindol-1-one,
2-(2-amino-ethyl)-6-(4-trifluoromethyl-benzyloxy)-2,3-dihydro-isoindol-1-one 1:1 hydrochloride, and
2-(2-amino-ethyl)-6-(4-trifluoromethyl-benzyloxy)-2,3-dihydro-isoindol-1-one 1:1 hydrochloride,
   or pharmaceutically acceptable salts thereof.

[49] In another embodiment, the MAO-B inhibitor is selected from the group of compounds described U.S. Pat. No. 6,900,354 or pharmaceutically acceptable salts thereof. Compounds from this publication include compounds of formula XXVIII and XXIX, or pharmaceutically acceptable salts thereof:

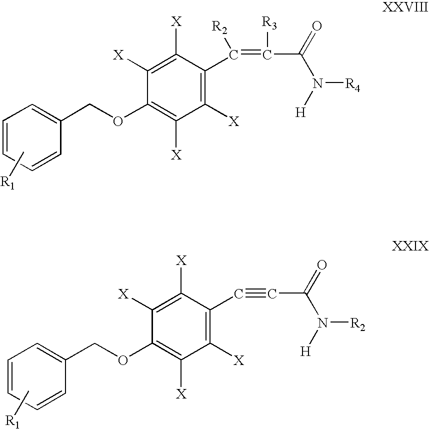

wherein:

X is selected from H and F;

R₁is selected from H, halogen, C₁₋₆ alkyl, C₁₋₆ haloalkyl, —CN, C₁₋₆ alkoxy, and C₁₋₆ haloalkoxy;

R₂ is selected from H and C₁₋₆ alkyl;

R₃is selected from H and C₁₋₆ alkyl; and,

R₄ is selected from H and C₁₋₆ alkyl.

[50] Examples of compounds of formula XXVIII and XXIX include:
N-methyl-3-[4-(4-methyl-benzyloxy)-phenyl]-acrylamide;
3-[4-(3-methoxy-benzyloxy)-phenyl]-N-methyl-acrylamide;
3-[4-(3-fluoro-benzyloxy)-phenyl]-2,N-dimethyl-acrylamide;
3-[4-(3-fluoro-benzyloxy)-phenyl]-N-methyl-acrylamide;
N-methyl-3-[4-(4-trifluoromethyl-benzyloxy)-phenyl]-acrylamide;
3-[4-(3,4-difluoro-benzyloxy)-phenyl]-N-methyl-acrylamide;
3-[4-(4-fluoro-benzyloxy)-phenyl]-N-methyl-acrylamide;
3-[4-(3-fluoro-benzyloxy)-phenyl]-2,N-dimethyl-propionamide;
3-[4-(3,4-difluoro-benzyloxy)-phenyl]-propionamide;
3-[4-(3-fluoro-benzyloxy)-phenyl]-N-methyl-butyramide;
3-[4-(3-Fluoro-benzyloxy)-phenyl]-propynoic acid methylamide;
3-[4-(3-fluoro-benzyloxy)-phenyl]-2-methyl-acrylamide;
3-[4-(3-fluoro-benzyloxy)-phenyl]-2,N-dimethyl-propionamide; and,
3-[4-(3-fluoro-benzyloxy)-phenyl]-propynoic acid amide;
   or pharmaceutically acceptable salts thereof.

[51] In another embodiment, the MAO-B inhibitor is selected from the group of compounds described U.S. Pat. No. 6,903,095 or pharmaceutically acceptable salts thereof. Compounds from this publication include compounds of formula XXX or pharmaceutically acceptable salts thereof:

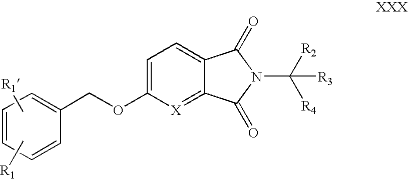

wherein:

X is selected from N and CH;

R₁ and R₁′ are independently selected from H, halogen, C₁₋₆ alkyl, C₁₋₆ haloalkyl, —CN, C₁₋₆ alkoxy, C₁₋₆ haloalkoxy, and CF₃;

R₂ is selected from H, (CH₂)ₙCN, (CH₂)ₙOR₄, (CH₂)ₙCON(R₄)₂, (CH₂)ₙCO₂R₄, CHR₅(CH₂)ₙOR₆, and (CH₂)ₙN(R₄)₂;

R₃is selected from H, C₁₋₆ alkyl, OH, (CH₂)ₙO—C₁₋₆ alkyl, (CH₂)ₙS—C₁₋₆ alkyl, (CH₂)ₙS(O)—C₁₋₆ alkyl, benzyl, and C₁₋₆ haloalkoxy;

R₄ is selected from H and C₁₋₆ alkyl;

R₅ is selected from H, C₁₋₆ alkyl, —CN, and CONH₂;

R₆ is selected from H and C₁₋₆ alkyl; and,

n is selected from 0, 1, 2, and 3.

[52] Examples of compounds of formula XXX include:
(S)-2-[5-(4-Fluoro-benzyloxy)-1,3-dioxo-1,3-dihydro-isoindol-2-yl]-propionamide;
2-(2-Amino-ethyl)-5-(4-fluoro-benzyloxy)-isoindole-1,3-dione 1:1 hydrochloride;
4-[5-(4-Fluoro-benzyloxy)-1,3-dioxo-1,3-dihydro-isoindol-2-yl]-3-hydroxy-butyramide;
(S)-5-(3-Methoxy-benzyloxy)-2-(2-methoxy-1-methyl-ethyl)-isoindole-1,3-dione;
(S)-4-[2-(2-Methoxy-1-methyl-ethyl)-1,3-dioxo-2,3-dihydro-1H-isoindol-5-yloxymethyl]-benzonitrile;
2-(2-Ethanesulfinyl-ethyl)-5-(4-fluoro-benzyloxy)-isoindole-1,3-dione;
(S)-2-(2-Methoxy-1-methyl-ethyl)-5-(4-trifluoromethoxy-benzyloxy)-isoindole-1,3-dione;
5-(4-Fluoro-benzyloxy)-2-thiophen-2-ylmethyl-isoindole-1,3-dione;
2-(2-Ethanesulfinyl-ethyl)-5-(4-fluoro-benzyloxy)-isoindole-1,3-dione;
(5-(4-Fluoro-benzyloxy)-2-(3 ,3 ,3-trifluoro-2-methoxy-propyl)-isoindole-1,3-dione;
(S)-2-[5-(3-Fluoro-benzyloxy)-1,3-dioxo-1,3-dihydro-isoindol-2-yl]-propionamide;
2-(2-Amino-ethyl)-5-(3-fluoro-benzyloxy)-isoindole-1,3-dione 1:1 hydrochloride;
2-[5-(4-fluoro-benzyloxy)-1,3-dioxo-1,3-dihydro-isoindol-2-yl]-acetamide;
(S)-2-[5-(4-fluoro-benzyloxy)-1,3-dioxo-1,3-dihydro-isoindol-2-yl]-propionamide;
(S)-2-[5-(4-fluoro-benzyloxy)-1,3-dioxo-1,3-dihydro-isoindol-2-yl]-3-hydroxy-propionamide;
(R)-2-[5-(4-fluoro-benzyloxy)-1,3-dioxo-1,3-dihydro-isoindol-2-yl]-propionamide;
2-[5-(3-fluoro-benzyloxy)-1,3-dioxo-1,3-dihydro-isoindol-2-yl]-propionamide;
(2-[5-(3-fluoro-benzyloxy)-1,3-dioxo-1,3-dihydro-isoindol-2-yl]-acetamide;
2-[5-(3-fluoro-benzyloxy)-1,3-dioxo-1,3-dihydro-isoindol-2-yl]-3-hydroxy-propionamide;
5-(4-fluoro-benzyloxy)-2-(2-hydroxy-ethyl)-isoindole-1,3-dione;
5-(4-fluoro-benzyloxy)-2-(2-methoxy-ethyl)-isoindole-1,3-dione;
5-(3-fluoro-benzyloxy)-2-(2-methoxy-ethyl)-isoindole-1,3-dione;
(S)-5-(4-fluoro-benzyloxy)-2-(2-methoxy-1-methyl-ethyl)-isoindole-1,3-dione;
(S)-5-(3-fluoro-benzyloxy)-2-(2-methoxy-1-methyl-ethyl)-isoindole-1,3-dione;
(S)-5-(2-fluoro-benzyloxy)-2-(2-methoxy-1-methyl-ethyl)-isoindole-1,3-dione;
(S)-2-(2-methoxy-1-methyl-ethyl)-5-(4-trifluoromethyl-benzyloxy)-isoindole-1,3-dione;
(S)-5-(4-bromo-benzyloxy)-2-(2-methoxy-1-methyl-ethyl)-isoindole-1,3-dione;
(S)-5-(3,4-difluoro-benzyloxy)-2-(2-methoxy-1-methyl-ethyl)-isoindole-1,3-dione;
5-(3-fluoro-benzyloxy)-2-(2-hydroxy-ethyl)-isoindole-1,3-dione;
5-(4-fluoro-benzyloxy)-2-(3,3,3-trifluoro-2-hydroxy-propyl)-isoindole-1,3-dione;
5-(3,5-bis-trifluoromethyl-benzyloxy)-2-(2-methoxy-1-methyl-ethyl)-isoindole-1,3-dione;
2-(2-ethylsulfanyl-ethyl)-5-(4-fluoro-benzyloxy)-isoindole-1,3-dione;
(S)-2-[5-(4-fluoro-benzyloxy)-1,3-dioxo-1,3-dihydro-isoindol-2-yl]-thiopropionamide;
2-(2-ethylsulfanyl-ethyl)-5-(3-fluoro-benzyloxy)-isoindole-1,3-dione;
5-(4-fluoro-benzyloxy)-1,3-dioxo-1,3-dihydro-isoindol-2-yl]-acetonitrile; and,
[5-(3-fluoro-benzyloxy)-1,3-dioxo-1,3-dihydro-isoindol-2-yl]-acetonitrile;
   or pharmaceutically acceptable salts thereof.

[53] In another embodiment, the MAO-B inhibitor is selected from the group of compounds described U.S. Pat. No. 6,951,884 or pharmaceutically acceptable salts thereof. Compounds from this publication include compounds of formula XXXI or pharmaceutically acceptable salts thereof:

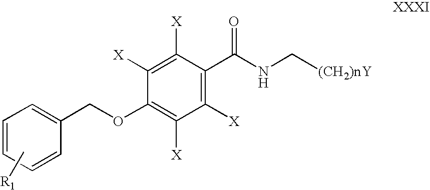

wherein:

X is selected from H and F;

Y is selected from NH₂, —CN, OH, C₁₋₆ alkoxy, and CON(R₂)₂;

R₁is selected from H, halogen, C₁₋₆ alkyl, C₁₋₆ haloalkyl, —CN, C₁₋₆ alkoxy, and C₁₋₆ haloalkoxy; and,

n is selected from 0, 1, and 2.

[54] Examples of compounds of formula XXXI include:
(S)-N-(1-carbamoyl-ethyl)-2-fluoro-4-(3-fluoro-benzyloxy)-benzamide;
2-[4-(3-fluorobenzyloxy)-2-fluoro-benzamido]acetamide;
(S)-N-(1-carbamoyl-2-hydroxy-ethyl)-2-fluoro-4-(3-fluoro-benzyloxy)-benzamide;
(R)-N-(1-carbamoyl-ethyl)-2-fluoro-4-(3-fluoro-benzyloxy)-benzamide;
2-[4-(4-fluorobenzyloxy)-2-fluoro-benzamido]acetamide;
(S)-N-(1-carbamoyl-ethyl)-2-fluoro-4-(4-fluoro-benzyloxy)-benzamide;
S)-2-Fluoro-4-(3-fluoro-benzyloxy)-N-(2-methoxy-1-methyl-ethyl)-benzamide;
2-fluoro-4-(3-fluoro-benzyloxy)-N-(2-methoxy-ethyl)-benzamide;
2-fluoro-4-(3-fluoro-benzyloxy)-N-(2-hydroxy-ethyl)-benzamide;
S)-N-(1-carbamoyl-ethyl)-3-fluoro-4-(4-fluoro-benzyloxy)-benzamide;
2-[4-(4-fluorobenzyloxy)-3-fluoro-benzamido]acetamide;
(S)-N-(1-carbamoyl-2-hydroxy-ethyl)-3-fluoro-4-(4-fluoro-benzyloxy)-benzamide;
2-[4-(3-fluorobenzyloxy)-3-fluoro-benzamido]acetamide;
(S)-N-(1-carbamoyl-ethyl)-3-fluoro-4-(3-fluoro-benzyloxy)-benzamide;
(R)-N-(1-carbamoyl-ethyl)-3-fluoro-4-(3-fluoro-benzyloxy)-benzamide;
(S)-N-(1-carbamoyl-2-hydroxy-ethyl)-3-fluoro-4-(3-fluoro-benzyloxy)-benzamide;
2-[4-(4-trifluoromethylbenzyloxy)-3-fluoro-benzamido]acetamide;
(S)-N-(1-carbamoyl-2-hydroxy-ethyl)-3-fluoro-4-(4-trifluoromethyl-benzyloxy)-benzamide;
(S)-N-(1-carbamoyl-ethyl)-2,6-difluoro-4-(4-fluoro-benzyloxy)-benzamide;
N-carbamoylmethyl-2,6-difluoro-4-(4-fluoro-benzyloxy)-benzamide;
N-cyanomethyl-2,6-difluoro-4-(4-fluoro-benzyloxy)-benzamide;
2,6-difluoro-4-(4-fluoro-benzyloxy)-N-(2-methoxy-ethyl)-benzamide;
(S)-2,6-difluoro-4-(4-fluoro-benzyloxy)-N-(2-hydroxy-1-methyl-ethyl)-benzamide; and,
2,6-difluoro-4-(3-fluoro-benzyloxy)-N-(2-methoxy-ethyl)-benzamide;
   or pharmaceutically acceptable salts thereof.

[55] In another embodiment, the MAO-B inhibitor is selected from the group of compounds described U.S. Pat. No. 5,486,541 or pharmaceutically acceptable salts thereof. Compounds from this publication include compounds of formula XXXII or stereoisomers or pharmaceutically acceptable salts thereof:

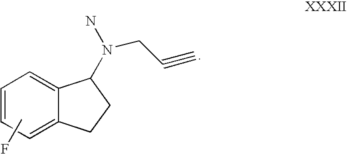

[56] Examples of compounds of formula XXXII include:
4-fluoro-N-propargyl-1-aminoindan;
5-fluoro-N-propargyl-1-aminoindan;
6-fluoro-N-propargyl-1-aminoindan;
(+)-6-fluoro-N-propargyl-1-aminoindan; and,
   or stereoisomers or pharmaceutically acceptable salts thereof.

[57] In another embodiment, the MAO-B inhibitor is selected from the group of compounds described U.S. Pat. No. 4,476,136 or pharmaceutically acceptable salts thereof. Compounds from this publication include compounds of formula XXXIII or pharmaceutically acceptable salts thereof:

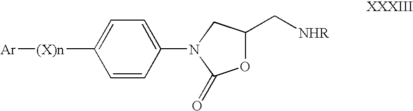

wherein:

X is selected from —CH₂CH₂—, —CH═CH—, —C≡C—, and —CH₂O—, where the CH₂ portion of CH₂O is linked to Ar;

Ar is selected from phenyl; phenyl substituted by a halogen atom or CF₃, and 3-chloro-4-fluoro-phenyl;

R is selected from H, C₁₋₆ alkyl, C₁₋₆ alkenyl, and C₁₋₆ alkynyl; and,

n is selected from 1, 2, and 3.

[58] Examples of compounds of formula XXXIII include:
5-Aminomethyl-3-{4-[2-(3-chloro-phenyl)-ethyl]-phenyl}-oxazolidin-2-one;
5-Aminomethyl-3-{4-[2-(3-chloro-phenyl)-vinyl]-phenyl}-oxazolidin-2-one;
5-Aminomethyl-3-{4-[2-(3-fluoro-phenyl)-ethyl]-phenyl}-oxazolidin-2-one;
3-{4-[2-(3-Fluoro-phenyl)-ethyl]-phenyl}-5-methylaminomethyl-oxazolidin-2-one;
3-{4-[2-(3-Chloro-phenyl)-vinyl]-phenyl}-5-methylaminomethyl-oxazolidin-2-one;
3-[4-(3-Chloro-benzyloxy)-phenyl]-5-propylaminomethyl-oxazolidin-2-one; and,
3-[4-(3-Chloro-benzyloxy)-phenyl]-5-ethynylaminomethyl-oxazolidin-2-one;
   or pharmaceutically acceptable salts thereof.

[59] In another embodiment, the MAO-B inhibitor is selected from the group of compounds described U.S. Pat. No. 4,897,409 or pharmaceutically acceptable salts thereof. Compounds from this publication include compounds of formula XXXIV or stereoisomers or pharmaceutically acceptable salts thereof:

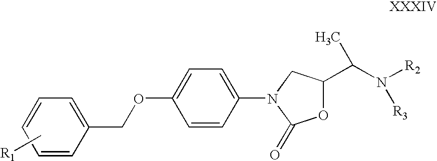

wherein:

R₁is selected from H, C₁₋₄ alkoxy, CF₃, and one or two halogen atoms; and

R₂ and R₃are independently selected from H and C₁₋₄ alkyl.

[60] Examples of compounds of formula XXXIV include:
3-[4-(3-chlorobenzyloxy) phenyl]-5-(1-dimethylaminoethyl)-oxazolidin-2-one;
3-[4-(3-methoxybenzyloxy) phenyl]-5-(1-dimethylaminoethyl)-oxazolidin-2-one;
3-[4-(3-chlorobenzyloxy) phenyl]-5-(1-methylaminoethyl)-oxazolidin-2-one;
3-[4-(3-methoxybenzyloxy) phenyl]-5-(1-methylaminoethyl)-oxazolidin-2-one;
3-[4-(3-chlorobenzyloxy) phenyl]-5-(1-aminoethyl)-oxazolidin-2-one; and,
3-[4-(3-methoxybenzyloxy) phenyl]-5-(1-aminoethyl)-oxazolidin-2-one;
   or stereoisomers or pharmaceutically acceptable salts thereof.

[61] In another embodiment, the MAO-B inhibitor is selected from the group of compounds described in US 2004/0106650, US 2004/0097578, and US 2004/0116707. Compounds from these publications include compounds of formula XXXV or pharmaceutically acceptable salts thereof:

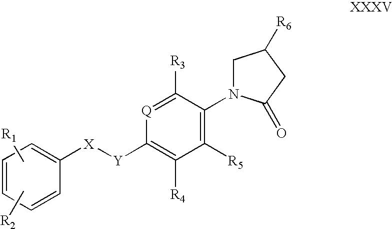

wherein:

Q is selected from N or CR₇;

X-Y is selected from —CH₂CH₂—, —CH═CH—, and —CH₂O—;

R₁and R₂ are selected from H, halogen, —CN, C₁₋₆ alkyl, C₁₋₆ haloalkyl, —CN, C₁₋₆ alkoxy, and C₁₋₆ haloalkoxy;

R₃, R₄, and R₅are selected from H and halogen;

R₇ is selected from H, halogen, and methyl;

R₆ is selected from NHR₉, CONHR, —CN, and CH₂CN; and,

R₉ is selected from C(O)H, C(O)C₁₋₃-alkyl, C(O)-halo-C₁₋₃ alkyl, C(O)OC₁₋₃-alkyl, CONH₂, and SO₂—C₁₋₃-alkyl.

[62] Examples of compounds of formula XXXV include:
(RS)-1-[4-(3-fluoro-benzyloxy)-phenyl]-5-oxo-pyrrolidine-3-carboxylic acid methylamide,
(RS)-[1-[4-(4-fluoro-benzyloxy)-phenyl]-5-oxo-pyrrolidine-3-carboxylic acid methylamide,
(RS)-1-[4-(3-chloro-benzyloxy)-phenyl]-5-oxo-pyrrolidine-3-carboxylic acid methylamide,
(RS)-[1-[4-(3,4-difluoro-benzyloxy)-phenyl]-5-oxo-pyrrolidine-3-carboxylic acid methylamide,
(RS)-[1-[4-(2,6-difluoro-benzyloxy)-phenyl]-5-oxo-pyrrolidine-3-carboxylic acid methylamide,
(RS)-5-oxo-1-[4-(2,4,6-trifluoro-benzyloxy)-phenyl]-pyrrolidine-3-carboxylic acid methylamide,
(RS)-5-oxo-1-[4-(2,4,5-trifluoro-benzyloxy)-phenyl]-pyrrolidine-3-carboxylic acid methylamide,
(RS)-5-oxo-1-[4-(2,3,6-trifluoro-benzyloxy)-phenyl]-pyrrolidine-3-carboxylic acid methylamide,
(RS)-5-oxo-1-[4-(2,3,4-trifluoro-benzyloxy)-phenyl]-pyrrolidine-3-carboxylic acid methylamide,
(RS)-5-oxo-1-[4-(3,4,5-trifluoro-benzyloxy)-phenyl]-pyrrolidine-3-carboxylic acid methylamide.
(RS)-1-[4-(5-fluoro-2-methyl-benzyloxy)-phenyl]-5-oxo-pyrrolidine-3-carboxylic acid methylamide,
(RS)-1-[4-(3-methoxy-benzyloxy)-phenyl]-5-oxo-pyrrolidine-3-carboxylic acid methylamide,
(RS)-1-[4-(2-methoxy-benzyloxy)-phenyl]-5-oxo-pyrrolidine-3-carboxylic acid methylamide,
(RS)-5-oxo-1-[4-(3-trifluoromethoxy-benzyloxy)-phenyl]-pyrrolidine-3-carboxylic acid methylamide,
(RS)-5-oxo-1-[4-(3-trifluoromethyl-benzyloxy)-phenyl]-pyrrolidine-3-carboxylic acid methylamide,
(RS)-1-[4-(3-cyano-benzyloxy)-phenyl]-5-oxo-pyrrolidine-3-carboxylic acid methylamide,
(RS)-1-[4-(3-fluoro-benzyloxy)-3-methyl-phenyl]-5-oxo-pyrrolidine-3-carboxylic acid methylamide,
(RS)-1-[4-(4-fluoro-benzyloxy)-3-methyl-phenyl]-5-oxo-pyrrolidine-3-carboxylic acid methylamide,
(RS)-1-[4-(3-chloro-benzyloxy)-3-methyl-phenyl]-5-oxo-pyrrolidine-3-carboxylic acid methylamide,
(RS)-1-[3-fluoro-4-(3-fluoro-benzyloxy)-phenyl]-5-oxo-pyrrolidine-3-carboxylic acid methylamide,
(RS)-1-[2-fluoro-4-(3-fluoro-benzyloxy)-phenyl]-5-oxo-pyrrolidine-3-carboxylic acid methylamide,
(RS)-1-[2,5-difluoro-4-(3-fluoro-benzyloxy)-phenyl]-5-oxo-pyrrolidine-3-carboxylic acid methylamide
(RS)-1-(4-benzyloxy-phenyl)-5-oxo-pyrrolidine-3-carboxylic acid methylamide,
(R)-1-[4-(3-fluoro-benzyloxy)-phenyl]-5-oxo-pyrrolidine-3-carboxylic acid methylamide,
(S)-1-[4-(3-fluoro-benzyloxy)-phenyl]-5-oxo-pyrrolidine-3-carboxylic acid methylamide,
(R)-1-(4-benzyloxy-phenyl)-5-oxo-pyrrolidine-3-carboxylic acid methylamide,
(S)-1-(4-benzyloxy-phenyl)-5-oxo-pyrrolidine-3-carboxylic acid methylamide,
(R)-1-[4-(4-fluoro-benzyloxy)-phenyl]-5-oxo-pyrrolidine-3-carboxylic acid methylamide,
(R)-1-[4-(3-fluoro-benzyloxy)-phenyl]-5-oxo-pyrrolidine-3-carboxylic acid methylamide,
(R)-1-[4-(3-chloro-benzyloxy)-phenyl]-5-oxo-pyrrolidine-3-carboxylic acid methylamide,
(R)-1-[4-(2,6-difluoro-benzyloxy)-phenyl]-5-oxo-pyrrolidine-3-carboxylic acid methylamide
(R)-5-oxo-1-[4-(2,4,6-trifluoro-benzyloxy)-phenyl]-pyrrolidine-3-carboxylic acid methylamide,
(RS)-1-[4-(3,4-difluoro-benzyloxy)-phenyl]-5-oxo-pyrrolidin-3-yl}-acetonitrile,
(RS)-{1-[4-(3-fluoro-benzyloxy)-phenyl]-5-oxo-pyrrolidin-3-yl}-acetonitrile,
(RS)-[1-(4-benzyloxy-phenyl)-5-oxo-pyrrolidin-3-yl]-acetonitrile,
(RS)-(E)-1-{4-[2-(3-fluoro-phenyl)-vinyl]-phenyl}-5-oxo-pyrrolidine-3-carboxylic acid methylamide,
(RS)-(E)-1-{4-[2-(4-methoxy-phenyl)-vinyl]-phenyl}-5-oxo-pyrrolidine-3-carboxylic acid methylamide,
(RS)-(E)-1-{4-[2-(3-methoxy-phenyl)-vinyl]-phenyl}-5-oxo-pyrrolidine-3-carboxylic acid methylamide,
(RS)-(E)-1-{4-[2-(4-fluoro-phenyl)-vinyl]-phenyl}-5-oxo-pyrrolidine-3-carboxylic acid methylamide
(RS)-1-{4-[2-(3-chloro-phenyl)-ethyl]-phenyl}-5-oxo-pyrrolidine-3-carboxylic acid methylamide,
(RS)-1-{4-[2-(4-chloro-phenyl)-ethyl]-phenyl}-5-oxo-pyrrolidine-3-carboxylic acid methylamide,
(RS)-1-{4-[2-(3-fluoro-phenyl)-ethyl]-phenyl}-5-oxo-pyrrolidine-3-carboxylic acid methylamide,
(RS)-1-{4-[2-(4-fluoro-phenyl)-ethyl]-phenyl}-5-oxo-pyrrolidine-3-carboxylic acid methylamide,
(RS)-1-{4-[2-(3-methoxy-phenyl)-ethyl]-phenyl}-5-oxo-pyrrolidine-3-carboxylic acid methylamide,
(RS)-1-[6-(4-fluoro-benzyloxy)-pyridin-3-yl]-5-oxo-pyrrolidine-3-carboxylic acid methylamide,
(RS)-1-[4-(2-fluoro-benzyloxy)-phenyl]-5-oxo-pyrrolidine-3-carboxylic acid methylamide,
(RS)-N-{1-[4-(3-fluoro-benzyloxy)-phenyl]-5-oxo-pyrrolidin-3-yl}-acetamide,
(S)-N-{1-[4-(3-fluoro-benzyloxy)-phenyl]-5-oxo-pyrrolidin-3-yl}-acetamide,
(R)-N-{1-[4-(3-fluoro-benzyloxy)-phenyl]-5-oxo-pyrrolidin-3-yl}-acetamide,
(RS)-N-{1-[4-(3-fluoro-benzyloxy)-phenyl]-5-oxo-pyrrolidin-3-yl}-formamide,
(S)-N-{1-[4-(3-fluoro-benzyloxy)-phenyl]-5-oxo-pyrrolidin-3-yl}-formamide,
(R)-N-{1-[4-(3-fluoro-benzyloxy)-phenyl]-5-oxo-pyrrolidin-3-yl}-formamide,
(RS)-{1-[4-(3-fluoro-benzyloxy)-phenyl]-5-oxo-pyrrolidin-3-yl}-carbamic acid methyl ester,
(RS)-{1-[4-(3-fluoro-benzyloxy)-phenyl]-5-oxo-pyrrolidin-3-yl}-urea,
(RS)-N-{1-[4-(3-fluoro-benzyloxy)-phenyl]-5-oxo-pyrrolidin-3-yl}-methanesulfonamide,
(S)-2-fluoro-N-{1-[4-(3-fluoro-benzyloxy)-phenyl]-5-oxo-pyrrolidin-3-yl}-acetamide,
(S)-2,2-difluoro-N-{1-[4-(3-fluoro-benzyloxy)-phenyl]-5-oxo-pyrrolidin-3-yl}-acetamide,
(S)-2,2,2-trifluoro-N-{1-[4-(3-fluoro-benzyloxy)-phenyl]-5-oxo-pyrrolidin-3-yl}-acetamide,
(RS)-N-{1-[4-(4-fluoro-benzyloxy)-phenyl]-5-oxo-pyrrolidin-3-yl}-acetamide,
(R)-N-{1-[4-(4-fluoro-benzyloxy)-phenyl]-5-oxo-pyrrolidin-3-yl}-acetamide,
(S)-N-{1-[4-(4-fluoro-benzyloxy)-phenyl]-5-oxo-pyrrolidin-3-yl}-acetamide,
(RS)-N-{1-[4-(4-fluoro-benzyloxy)-phenyl]-5-oxo-pyrrolidin-3-yl}-formamide,
(RS)-N-[1-(4-benzyloxy-phenyl)-5-oxo-pyrrolidin-3-yl]-acetamide,
(RS)-N-{1-[4-(2-fluoro-benzyloxy-phenyl]-5-oxo-pyrrolidin-3-yl}-acetamide,
(RS)-(E)-N-(1-{4-[2-(3-fluoro-phenyl)-vinyl]-phenyl}-5-oxo-pyrrolidin-3-yl)-acetamide,
(RS)-N-(1-{4-[2-(3-fluoro-phenyl)-ethyl]-phenyl}-5-oxo-pyrrolidin-3-yl)-acetamide,
(RS)-N-{1-[6-(4-fluoro-benzyloxy)-pyridin-3-yl]-5-oxo-pyrrolidin-3-yl}-acetamide,
(S)-N-{1-[4-(3-chloro-benzyloxy)-phenyl]-5-oxo-pyrrolidin-3-yl}-acetamide,
(S)-N-{1-[4-(2,6-difluoro-benzyloxy)-phenyl]-5-oxo-pyrrolidin-3-yl}acetamide,
(S)-N-{5-oxo-1-[4-(2,4,6-trifluoro-benzyloxy)-phenyl]-pyrrolidin-3-yl}-acetamide,
(S)-N-{1-[4-(3-methoxy-benzyloxy)-phenyl]-5-oxo-pyrrolidin-3-yl}-acetamide,
(S)-N-{5-oxo-1-[4-(4-trifluoromethyl-benzyloxy)-phenyl]-pyrrolidin-3-yl}-acetamide,
(S)-N-{1-[4-(4-methyl-benzyloxy)-phenyl]-5-oxo-pyrrolidin-3-yl}-acetamide,
(S)-N-{1-[4-(3-cyano-benzyloxy)-phenyl]-5-oxo-pyrrolidin-3-yl}-acetamide.
(RS)-1-(4-benzyloxy-phenyl)-2-oxo-pyrrolidine-3-carbonitrile,
(RS)-1-[4-(3-fluoro-benzyloxy)-phenyl]-2-oxo-pyrrolidine-3-carboxylic acid methylamide,
(RS)-1-[4-(3-fluoro-benzyloxy)-phenyl]-2-oxo-pyrrolidine-3-carboxylic acid amide,
(RS)-1-[4-(4-fluoro-benzyloxy)-phenyl]-2-oxo-pyrrolidine-3-carboxylic acid amide,
(RS)-1-[4-(4-fluoro-benzyloxy)-phenyl]-2-oxo-pyrrolidine-3-carboxylic acid methylamide,
(RS)-2-oxo-1-[4-(4-trifluoromethyl-benzyloxy)-phenyl]-pyrrolidine-3-carboxylic acid amide,
(RS)-2-oxo-1-[4-(4-trifluoromethyl-benzyloxy)-phenyl]-pyrrolidine-3-carboxylic acid methylamide,
(S)-N-[1-(4-benzyloxy-phenyl)-2-oxo-pyrrolidin-3-yl]-acetamide,
(S)-N-[1-(4-benzyloxy-phenyl)-2-oxo-pyrrolidin-3-yl]-methanesulfonamide,
(S)-N-{1-[4-(3-fluoro-benzyloxy)-phenyl]-2-oxo-pyrrolidin-3-yl}-acetamide,
(R)-N-{1-[4-(3-fluoro-benzyloxy)-phenyl]-2-oxo-pyrrolidin-3-yl}-acetamide,
(R)-N-{1-[4-(3-fluoro-benzyloxy)-phenyl]-2-oxo-pyrrolidin-3-yl}-methanesulfonamide,
(S)-N-{1-[4-(3-fluoro-benzyloxy)-phenyl]-2-oxo-pyrrolidin-3-yl}-methanesulfonamide,
(S)-{1-[4-(3-fluoro-benzyloxy)-phenyl]-2-oxo-pyrrolidin-3-yl}-carbamic acid methyl ester,
(R)-N-{1-[4-(3-fluoro-benzyloxy)-phenyl]-2-oxo-pyrrolidin-3-yl}-formamide,
(S)-N-{1-[4-(3-fluoro-benzyloxy)-phenyl]-2-oxo-pyrrolidin-3-yl}-formamide,
(R)-{1-[4-(3-fluoro-benzyloxy)-phenyl]-2-oxo-pyrrolidin-3-yl}-urea,
(S)-{1-[4-(3-fluoro-benzyloxy)-phenyl]-2-oxo-pyrrolidin-3-yl}-urea,
(S)-N-{1-(S)-[4-(4-fluoro-benzyloxy)-phenyl]-2-oxo-pyrrolidin-3-yl}-acetamide,
(S)-N-{1-(S)-[4-(2,6-difluoro-benzyloxy)-phenyl]-2-oxo-pyrrolidin-3-yl}-acetamide, and
(S)-N-{1-[4-(3,4-difluoro-benzyloxy)-phenyl]-2-oxo-pyrrolidin-3-yl}-acetamide,
   or pharmaceutically acceptable salts thereof.

[63] In another embodiment, the MAO-B inhibitor is selected from the group of compounds described in US 2004/0142951. Compounds from this publication include compounds of formula XXXVI or pharmaceutically acceptable salts thereof:

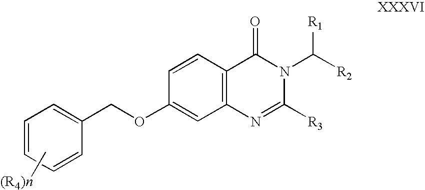

wherein:

R₁is selected from (CH₂)ₙCONR₅R₆, (CH₂)ₙCOOR₇, (CH₂)ₙNR₅R₆, (CH₂)ₙCN, (CH₂)ₙOR₈, and phenyl that is unsubstituted or substituted by 1-3substituents selected from halogen and fluoro-C₁₋₆ alkyl;

R₂ is selected from H, C₁₋₆ alkyl, and C₃₆-cycloalkyl;

R₃is selected from H, C₁₋₆ alkyl, C₃₋₆ cycloalkyl, and benzyl;

R₄ is selected from halogen, cyano, C₁₋₆ alkyl, C₁₋₆ fluorooalkyl, —CN, C₁₋₆ alkoxy, and C₁₋₆ fluorooalkoxy;

R₅and R₆ are independently selected from H and C₁₋₆ alkyl;

R₇ is selected from H and C₁₋₆ alkyl;

R₈ is C₁₋₆ alkyl;

m is selected from 1, 2 and 3; and,

n is selected from 0, 1, 2.

[64] Examples of compounds of formula XXXVI include:
2-[7-(3-fluoro-benzyloxy)-4-oxo-4H-quinazolin-3-yl]-acetamide,
2-[7-(3-fluoro-benzyloxy)-4-oxo-4H-quinazolin-3-yl]-propionamide,
2-[7-(4-fluoro-benzyloxy)-4-oxo-4H-quinazolin-3-yl]-acetamide,
2-[7-(4-fluoro-benzyloxy)-4-oxo-4H-quinazolin-3-yl]-propionamide,
2-[7-(3-fluoro-benzyloxy)-2-methyl-4-oxo-4H-quinazolin-3-yl]-acetamide,
2-[2-cyclopropyl-7-(3-fluoro-benzyloxy)-4-oxo-4H-quinazolin-3-yl]-acetamide,
7-(3-fluoro-benzyloxy)-3-(2-methoxy-ethyl)-3H-quinazolin-4-one,
7-(4-fluoro-benzyloxy)-3-(2-methoxy-ethyl)-3H-quinazolin-4-one,
7-(3-fluoro-benzyloxy)-3-(2-methoxy-ethyl)-2-methyl-3H-quinazolin-4-one,
3-(2-amino-ethyl)-7-(3-fluoro-benzyloxy)-3H-quinazolin-4-one 1:2 hydrochloride,
3-(3-amino-propyl)-7-(3-fluoro-benzyloxy)-3H-quinazolin-4-one 1:2 hydrochloride,
3-(2-amino-ethyl)-7-(4-fluoro-benzyloxy)-3H-quinazolin-4-one 1:1 hydrochloride,
2-[7-(3-fluoro-benzyloxy)-2-methyl-4-oxo-4H-quinazolin-3-yl]-ethyl-ammonium chloride,
[7-(3-fluoro-benzyloxy)-4-oxo-4H-quinazolin-3-yl]-acetic acid ethyl ester; fluoro-[7-(3-fluoro-benzyloxy)-4-oxo-4H-quinazolin-3-yl]-acetic acid ethyl ester;
2-[7-(3-fluoro-benzyloxy)-4-oxo-4H-quinazolin-3-yl]-propionic acid ethyl ester;
[7-(3-fluoro-benzyloxy)-4-oxo-4H-quinazolin-3-yl]-acetic acid tert-butyl ester;
2-[7-(3-fluoro-benzyloxy)-4-oxo-4H-quinazolin-3-yl]-propionic acid tert-butyl ester;
[7-(4-fluoro-benzyloxy)-4-oxo-4H-quinazolin-3-yl]-acetic acid ethyl ester;
2-[7-(4-fluoro-benzyloxy)-4-oxo-4H-quinazolin-3-yl]-propionic acid ethyl ester,
3-(3-fluoro-benzyl)-7-(3-fluoro-benzyloxy)-3H-quinazolin-4-one;
3-[7-(4-fluoro-benzyloxy)-4-oxo-4H-quinazolin-3-yl]-propionamide;
2-[7-(3-fluoro-benzyloxy)-2-isopropyl-4-oxo-4H-quinazolin-3-yl]-acetamide;
[7-(3-fluoro-benzyloxy)-2-isopropyl-4-oxo-4H-quinazolin-3-yl]-acetonitrile;
2-cyclopropyl-7-(3-fluoro-benzyloxy)-3-(2-methoxy-ethyl)-3H-quinazolin-4-one;
[2-cyclopropyl-7-(3-fluoro-benzyloxy)-4-oxo-4H-quinazolin-3-yl]-acetic acid methyl ester; and
2-[2-benzyl-7-(3-fluoro-benzyloxy)-4-oxo-4H-quinazolin-3-yl]-acetamide,
   or pharmaceutically acceptable salts thereof.

[65] In another embodiment, the MAO-B inhibitor is selected from the group of compounds described in US 2005/0107360. Compounds from this publication include compounds of formula XXXVII or pharmaceutically acceptable salts thereof:

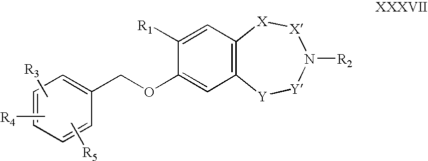

wherein:

R₁is selected from H and methyl;

R₂ is selected from H, C₁₋₃ alkyl, CH₂CONH₂, CH(CH₃)CONH₂, SO₂CH₃, and COR₆;

R₃, R₄, and R₅are independently selected from H, halogen, —CN, C₁₋₃ alkyl, and C₁₋₃ alkoxy;

R₆ is selected from H, methyl, CH₂OCH₃, CONH₂, CH₂CONH₂, OCH₃, NH₂, and NHCH₂CH₃;

X—X′ is selected from —CH₂CH₂—, —CH═CH—, and —CH₂CO—;

Y—Y′ is selected from —CH₂CH₂, —CH═CH—, and —CH₂CO—;

X—X′ is selected from —CH₂—, and Y—Y′ is CH₂CH₂CO—;

provided that:
a. when one of X—X′ and Y—Y′ is —CH₂CH₂— and the other is —CH═CH;
b. when both of X—X′ and Y—Y′ are —CH═CH—, then R₂ is SO₂CH₃ or —COR₆; or
c. when X—X′ and Y—Y′ are —CH₂CO—, then R₂ is H, C₁₋₃ alkyl, CH₂CONH₂, or CH(CH₃)CONH₂.

[66] Examples of compounds of formula XXXVII include:
1-[7-(3-fluoro-benzyloxy)-1,2,4,5-tetrahydro-benzo[d]azepin-3-yl]-ethanone,
1-[7-(3-fluoro-benzyloxy)-1,2,4,5-tetrahydro-benzo[d]azepin-3-yl]-2-methoxy-ethanone,
2-[7-(3-fluoro-benzyloxy)-1,2,4,5-tetrahydro-benzo[d]azepin--3-yl]-2-oxo-acetamide,
3-[7-(3-fluoro-benzyloxy)-1,2,4,5-tetrahydro-benzo-[d]azepin-3-yl]-3-oxo-propionamide,
7-(3-fluoro-benzyloxy)-1,2,4,5-tetrahydro-benzo[d]azepine-3-carboxylic acid methyl ester,
7-(3-fluoro-benzyloxy)-1,2,4,5-tetrahydro-benzo[d]azepine-3-carbaldehyde,
7-(3-fluoro-benzyloxy)-3-methanesulfonyl-2,3,4,5-tetrahydro-1H-benzo[d]azepine,
7-(3-fluoro-benzyloxy)-1,2,4,5-tetrahydro-benzo[d]azepine-3-carboxylic acid amide,
7-(3-fluoro-benzyloxy)-1,2,4,5-tetrahydro-benzo[d]azepine-3-carboxylic acid ethylamide.
2-[7-(3-fluoro-benzyloxy)-1,2,4,5-tetrahydro-benzo[d]azepin-3-yl]-acetamide,
(RS)-2-[7-(3-fluoro-benzyloxy)-1,2,4,5-tetrahydro-benzo[d]azepin-3-yl]-propionamide,
8-(3-fluoro-benzyloxy)-1,3-dihydro-benzo[d]azepin-2-one,
8-(3-fluoro-benzyloxy)-3-methyl-1,3-dihydro-benzo[d]azepin-2-one,
8-(3-fluoro-benzyloxy)-3-methoxyacetyl-1,3-dihydro-benzo[d]azepin-2-one,
3-acetyl-8-(3-fluoro-benzyloxy)-1,3-dihydro-benzo[d]azepin-2-one,
8-(3-fluoro-benzyloxy)-1,3,4,5-tetrahydro-benzo[d]azepin-2-one,
7-(2,3,4-trifluoro-benzyloxy)-1,3,4,5-tetrahydro-benzo[d]azepin-2-one.
7-(2,3,4-trifluoro-benzyloxy)-1,2,4,5-tetrahydro-benzo[c]azepin-3-one,
7-(2,6-difluoro-benzyloxy)-1,3,4,5-tetrahydro-benzo[d]azepin-2-one,
7-(2,6-difluoro-benzyloxy)-1,2,4,5-tetrahydro-benzo[c]azepin-3-one,
7-benzyloxy-1,3,4,5-tetrahydro-benzo[d]azepin-2-one,
7-(3-fluoro-benzyloxy)-1,3,4,5-tetrahydro-benzo[d]azepin-2-one,
7-(3-chloro-benzyloxy)-1,3,4,5-tetrahydro-benzo[d]azepin-2-one,
3-acetyl-7-(3-chloro-benzyloxy)-1,3,4,5-tetrahydro-benzo[d]azepin-2-one, and
7-(3-fluoro-benzyloxy)-1,2,4,5-tetrahydro-benzo[c]azepin-2-one,
   or pharmaceutically acceptable salts thereof.

[67] In another embodiment, the MAO-B inhibitor is selected from the group of compounds described in US 2004/0010038. Compounds from this publication include compounds of formula XXXVIII or pharmaceutically acceptable salts or stereoisomers thereof:

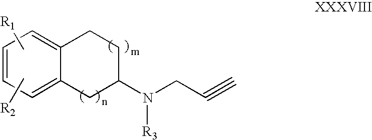

wherein:

R₁is OH or OC(O)R₄;

R₂ is OC(O)R₄ or H;

R₃is H or C₁₋₆ alkyl;

R₄ is selected from the group consisting of C₁₋₆ alkyl, C₆₋₁₂ aryl, C₆₋₁₂ aryl-C₁₋₆ alkylene, and, NR₅R₆;

R₅and R₆ are independently selected from the group consisting of H, C₁₋₈ alkyl, C₆₋₁₂ aryl, C₆₋₁₂ aryl-C₁₋₆ alkylene, and C₆₋₁₂ cycloalkyl, each optionally substitute with a group selected from halogen, C₁₋₆ alkyl, C₁₋₆ alkoxy, —CN, NO₂, and OH;

n is 0 or 1; and,

m is 1or 2.

In another embodiment, the present invention provides a method of treating obesity, comprising: administering to a patient in need thereof a therapeutically effective amount of:
(a) a MAO-B inhibitor according to the present invention or a stereoisomer or pharmaceutically acceptable salt thereof, and,
(b) a second component selected from an appetite suppressant and a gut lipase inhibitor.

In another embodiment, the present invention also provides a method of preventing or reversing the deposition of adipose tissue in a patient in need thereof by the administration of a compound of the present invention. By preventing or reversing the deposition of adipose tissue, compound of the present invention are expected to reduce the incidence or severity of obesity, thereby reducing the incidence or severity of associated co-morbidities.

In another embodiment, the present invention provides a compound of the present invention for use in therapy.

In another embodiment, the present invention provides the use of the present invention for the manufacture of a medicament for the treatment of obesity, diabetes, cardiometabolic disorders, and a combination thereof.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

Definitions

The examples provided in the definitions present in this application are non-inclusive unless otherwise stated. They include but are not limited to the recited examples.

The compounds herein described may have asymmetric centers, geometric centers (e.g., double bond), or both. All chiral, diastereomeric, racemic forms and all geometric isomeric forms of a structure are included, unless the specific stereochemistry or isomeric form is specifically indicated. Compounds of the present invention containing an asymmetrically substituted atom may be isolated in optically active or racemic forms. It is well known in the art how to prepare optically active forms, such as by resolution of racemic forms, by synthesis from optically active starting materials, or through use of chiral auxiliaries. Geometric isomers of olefins, C═N double bonds, or other types of double bonds may be present in the compounds described herein, and all such stable isomers are included in the present invention. Specifically, cis and trans geometric isomers of the compounds of the present invention may also exist and may be isolated as a mixture of isomers or as separated isomeric forms. All processes used to prepare compounds of the present invention and intermediates made therein are considered to be part of the present invention. All tautomers of shown or described compounds are also considered to be part of the present invention.

“Alkyl” includes both branched and straight-chain saturated aliphatic hydrocarbon groups having the specified number of carbon atoms. C₁₋₆ alkyl, for example, includes C₁, C₂, C₃, C₄, C₅, and C₆ alkyl groups. Examples of alkyl include methyl, ethyl, n-propyl, i-propyl, n-butyl, s-butyl, t-butyl, n-pentyl, and s-pentyl.

“Alkenyl” includes the specified number of hydrocarbon atoms in either straight or branched configuration with one or more unsaturated carbon-carbon bonds that may occur in any stable point along the chain, such as ethenyl and propenyl. C₂₋₆ alkenyl includes C₂, C₃, C₄, C₅, and C₆ alkenyl groups.

“Alkynyl” includes the specified number of hydrocarbon atoms in either straight or branched configuration with one or more triple carbon-carbon bonds that may occur in any stable point along the chain, such as ethynyl and propynyl. C₂₋₆ Alkynyl includes C₂, C₃, C₄, C₅, and C₆ alkynyl groups.

“Haloalkyl” includes both branched and straight-chain saturated aliphatic hydrocarbon groups having the specified number of carbon atoms, substituted with 1 or more halogens (for example —CᵥF_{w} where v=1 to 3 and w=1 to (2v+1)). Examples of haloalkyl include trifluoromethyl, trichloromethyl, pentafluoroethyl, and pentachloroethyl.

“Alkanoyl” includes an alkyl group as defined above attached through a carbonyl, wherein the alkyl-carbonyl has the indicated number of carbon atoms. C₁₋₆ alkanoyl includes C₁, C₂, C₃, C₄, and C₅alkyl groups attached to a carbonyl. Examples of alkanoyl include ethanoyl, n-propanoyl, n-butanoyl, and n-pentanoyl.

“Alkoxy” includes an alkyl group as defined above with the indicated number of carbon atoms attached through an oxygen bridge. C₁₋₆ alkoxy includes C₁, C₂, C₃, C₄, C₅, and C₆ alkoxy groups. Examples of alkoxy include methoxy, ethoxy, n-propoxy, i-propoxy, n-butoxy, s-butoxy, t-butoxy, n-pentoxy, and s-pentoxy.

“Haloalkoxy” includes a haloalkyl group as defined above with the indicated number of carbon atoms attached through an oxygen bridge. C₁₋₆ haloalkoxy includes C₁, C₂, C₃, C₄, C₅, and C₆ haloalkoxy groups. Examples of haloalkoxy include trifluoromethoxy, trichloromethoxy, pentafluoroethoxy, and pentachloroethoxy.

“Cycloalkyl” includes the specified number of hydrocarbon atoms in a saturated ring, such as cyclopropyl, cyclobutyl, cyclopentyl, cyclohexyl, cycloheptyl, and cyclooctyl. C₃₋₈ cycloalkyl includes C₃, C₄, C₅, C₆, C₇, and C₈ cycloalkyl groups.

Halo” or “halogen” refers to fluoro, chloro, bromo, and iodo.

The group “C₆H₄” represents a phenylene.

“Aryl” refers to any stable 6, 7, 8, 9, 10, 11, 12, or 13 membered monocyclic, bicyclic, or tricyclic ring, wherein at least one ring, if more than one is present, is aromatic. Examples of aryl include fluorenyl, phenyl, naphthyl, indanyl, adamantyl, and tetrahydronaphthyl.

“Heteroaryl” refers to any stable 5, 6, 7, 8, 9, 10, 11, or 12 membered monocyclic, bicyclic, or tricyclic heterocyclic ring that is aromatic, and which consists of carbon atoms and 1, 2, 3, or 4 heteroatoms independently selected from the group consisting of N, O, and S. If the heteroaryl group is bicyclic or tricyclic, then at least one of the two or three rings must contain a heteroatom, though both or all three may each contain one or more heteroatoms. If the heteroaryl group is bicyclic or tricyclic, then only one of the rings must be aromatic. The N group may be N, NH, or N-substituent, depending on the chosen ring and if substituents are recited. The nitrogen and sulfur heteroatoms may optionally be oxidized (e.g., S, S(O), S(O)₂, and N—O). The heteroaryl ring may be attached to its pendant group at any heteroatom or carbon atom that results in a stable structure. The heteroaryl rings described herein may be substituted on carbon or on a nitrogen atom if the resulting compound is stable.

Examples of heteroaryl includes acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolyl,1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, pyranyl, pyrazinyl, pyrazolyl, pyridazinyl, pyridooxazole, pyridoimidazole, pyridothiazole, pyridinyl, pyridyl, pyrimidinyl, 2H-pyrrolyl, pyrrolyl, quinazolinyl, quinolinyl, 4H-quinolizinyl, quinoxalinyl, quinuclidinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preventing the deposition of adipose tissue covers methods of treating wherein the levels of adipose tissue of a subject remain about the same as prior to being treated in accordance with the present invention (i.e., its pre-administration level) or not more than about 1, 2, 3, 4, 5, 6, 7, 8, 9, or 10% greater than pre-administration level (particularly when the subject is pre-disposed to increasing adipose tissue levels).

Reversing the deposition of adipose tissue covers methods of treating wherein the levels of adipose tissue of a subject are lower than those prior to being treated in accordance with the present invention (i.e., its pre-administration level). Examples of lower include 1, 2, 3, 4, 5, 6, 7, 8, 9, 10, 11, 12, 13, 14, 15, 16, 17, 18, 19, 20% or more lower than pre-administration level.

Mammal and patient covers warm blooded mammals that are typically under medical care (e.g., humans and domesticated animals). Examples of mammals include (a) feline, canine, equine, bovine, and human and (b) human.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p 1445, the disclosure of which is hereby incorporated by reference.

“Therapeutically effective amount” includes an amount of a compound of the present invention that is effective when administered alone or in combination to treat obesity or another indication listed herein. “Therapeutically effective amount” also includes an amount of the combination of compounds claimed that is effective to treat the desired indication. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

Utility

Obesity is defined as having a body mass index (BMI) of 30 or above. The index is a measure of an individual's body weight relative to height. BMI is calculated by dividing body weight (in kilograms) by height (in meters) squared. Normal and healthy body weight is defined as having a BMI between 20 and 24.9. Overweight is defined as having a BMI≧25. Obesity has reached epidemic proportions in the U.S., with 44 million obese Americans, and an additional eighty million deemed medically overweight.

Obesity is a disease characterized as a condition resulting from the excess accumulation of adipose tissue, especially adipose tissue localized in the abdominal area. It is desirable to treat overweight or obese patients by reducing their amount of adipose tissue, and thereby reducing their overall body weight to within the normal range for their sex and height. In this way, their risk for co-morbidities such as diabetes and cardiovascular disease will be reduced. It is also desirable to prevent normal weight individuals from accumulating additional, excess adipose tissue, effectively maintaining their body weights at a BMI<25and preventing the development of co-morbidities. It is also desirable to control obesity, effectively preventing overweight and obese individuals from accumulating additional, excess adipose tissue, reducing the risk of further exacerbating their co-morbidities.

There exist two forms of MAO, designated MAO-A and MAO-B. The two forms differ with respect to substrate and inhibitor specificities and amino acid number and sequence. A preferred substrate for MAO-B is beta-phenylethylamine. In contrast, a preferred substrate for MAO-A is serotonin. Some MAO inhibitors show selectivity for MAO-A or for MAO-B, whereas other MAO inhibitors show little, if any selectivity. For example, the MAO inhibitor clorgyline preferentially inhibits MAO-A; the MAO inhibitor L-selegiline preferentially inhibits MAO-B; and, the MAO inhibitor iproniazid is non-selective (i.e., has a similar affinity for both). Examples of selectivity include a compound having about 2, 3, 4, 5, 6, 7, 8, 9, 10, 20, 30, 40, 50, 100, 200, 300, 400, 500, 600, 700, 800, 900, 1000, or more fold higher affinity for one form of MAO than for the other form. One of ordinary skill in the art recognizes that there can be some difficulty in classifying MAO inhibitors. Some compounds may selectively inhibit one form of MAO in vitro and then lose their selectivity in vivo. Also, selectivity of a compound may vary from species to species or from tissue to tissue. In the context of the present invention, it is desirable to inhibit MAO-B activity in vivo in a mammal. Thus, selectivity and affinity are based on the in vivo activity of the MAO inhibitor and the mammalian species to which it is being or to be administered. Examples of the selectivity of a MAO-B inhibitor of the present invention include (a) at least a 2, 3, 4, 5, 6, 7, 8, 9, 10, 20, 30, 40, 50, to 100-fold greater affinity for MAO-B than MAO-A in the mammalian species (e.g., human) to be treated and (b) at least 100-fold greater affinity for MAO-B than MAO-A in the mammalian species (e.g., human) to be treated.

As is known in the art, some MAO inhibitors such as iproniazid are non-selective in that they inhibit both MAO-A and MAO-B. Such non-selective inhibitors could also be used to practice the present invention is dosed at sufficient levels to inhibit MAO-B activity. Even though MAO-B selective compounds are preferred, one skilled in the art recognizes that even a drug that preferentially inhibits MAO-A, if dosed sufficiently, may also inhibit sufficient MAO-B. Thus, MAO-A inhibitors are also considered to be useful to practice the present invention when administered at a dosage sufficient enough to inhibit MAO-B activity.

The structures of some of the compounds described above are provided below.

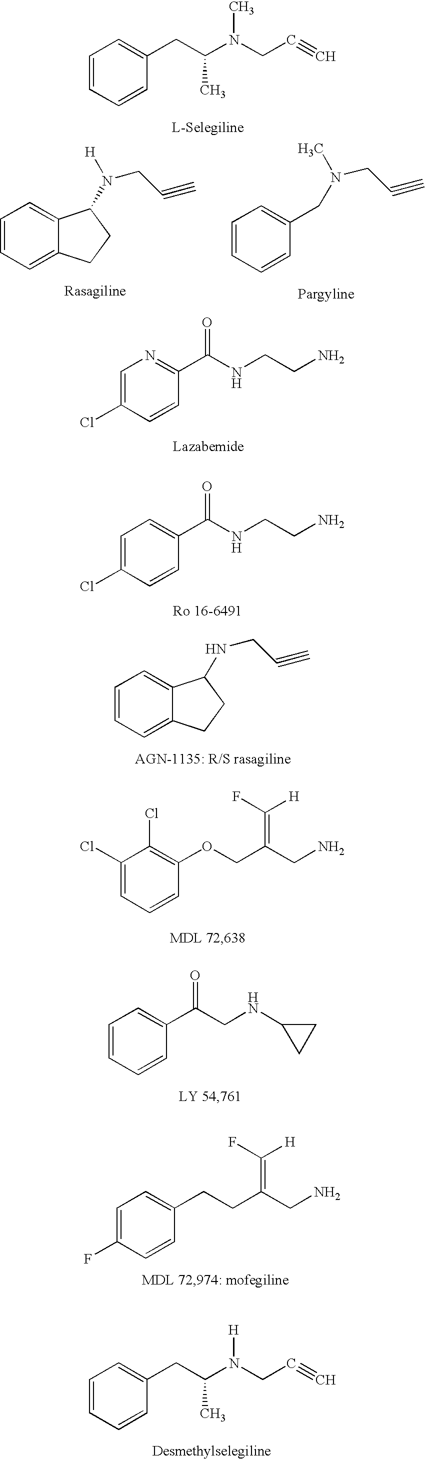

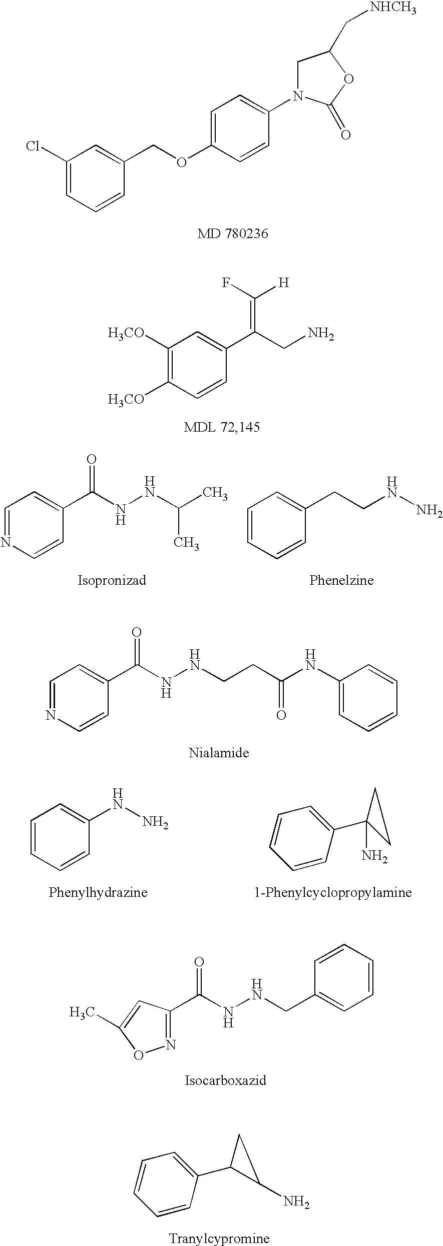

Most methods of treating obesity are dependent on a significant reduction in energy intake, either by a decrease in food intake (e.g., sibutramine) or by inhibition of fat absorption (e.g., orlistat). In the present invention, it can be desirable for adipose tissue to be significantly reduced in the absence of a significant reduction in food intake. The weight loss, as a result of the present invention, comes from the treatment with an MAO-B inhibitor, largely independent of appetite and food intake. Examples of the level of food intake during adipose tissue loss include (a) food intake is maintained, increased or about 0, 1, 2, 3, 4, 5, 6, 7, 8, 9, 10, 11, 12, 13, 14, 15, 16, 17, 18, 19, or 20% below the normal range of the subject prior to being treated in accordance with the present invention (i.e., its pre-administration level); (b) food intake is maintained, increased, or about 0, 1, 2, 3, 4, 5, 6, 7, 8, 9, 10, 11, 12, 13, 14, or 15% below its pre-administration level; (c) food intake is maintained, increased or about 0, 1, 2, 3, 4, 5, 6, 7, 8, 9, or 10% below its pre-administration level; and (d) food intake level is maintained, increased or about 0, 1, 2, 3, 4, or 5% below its pre-administration level.

In some cases, loss of adipose tissue can be accompanied by a concomitant loss of lean muscle mass. This is particularly evident in cancer patients who show a wasting of all body tissue components, including adipose tissue and lean muscle mass. In the present invention, however, it can be desirable for body fat to be significantly reduced in the absence of a significant reduction in lean body mass. Adipose tissue loss comes from treatment with an MAO-B inhibitor, independent of a significant change in lean body mass. Examples of the level of lean body mass during adipose tissue loss include (a) lean body mass is maintained, increased, or is no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% below the normal range of the subject prior to being treated in accordance with the present invention (i.e., its pre-administration level); (b) lean body mass is maintained, increased, or is no more than about 1, 2, 3, 4, 5, 6, 7, 8, 9, 10, 11, 12, 13, 14, or 15% below pre-administration levels; (c) lean body mass is maintained, increased, or is no more than about 1, 2, 3, 4, 5, 6, 7, 8, 9, or 10% below pre-administration levels; and (d) lean body mass is maintained, increased, or is no more than about 1, 2, 3, 4, or 5% below pre-administration levels.

In some cases, loss of adipose tissue can be accompanied by a concomitant loss of water mass. This is particularly evident with diet regimens that promote dehydration. In the present invention, it can be desirable for body fat to be significantly reduced in the absence of a significant reduction in water mass. In other words, adipose tissue loss comes from treatment with an MAO-B inhibitor, independent of a significant change in water mass. Examples of the level of water mass during adipose tissue loss include (a) water mass is maintained, increased, or is no more than about 1, 2, 3, 4, 5, 6, 7, 8, 9, 10, 11, 12, 13, 14, 15, 16, 17, 18, 19, 20, 21, 22, 23, 24, 25, 26, 27 , 28, 29, or 30% below the normal range of the subject prior to being treated in accordance with the present invention (i.e., its pre-administration level); (b) water mass is maintained, increased, or is no more than about 1, 2, 3, 4, 5, 6, 7, 8, 9, 10, 11, 12, 13, 14, or 15% below pre-administration levels; (c) water mass is maintained, increased, or is no more than about 1, 2, 3, 4, 5, 6, 7, 8, 9, or 10% below pre-administration levels; and (d) water mass is maintained, increased, or is no more than about 1, 2, 3, 4, or 5% below pre-administration levels.

Sibutramine and orlistat are currently marketed for use in the treatment of obesity. Although these two compounds share in common the same overall goal of reducing body weight secondary to reducing the amount of calories that reach the systemic circulation, they achieve weight loss through entirely different mechanisms. Sibutramine, a CNS appetite suppressant, inhibits the neuronal reuptake of serotonin and noradrenaline. Orlistat inhibits gut lipase enzymes that are responsible for breaking down ingested fat.

The mechanism of action of MAO-B inhibitors is believed to be entirely different from appetite suppressants, gut lipase inhibitors, and other agents with similar indications (e.g., serotonin agonists, leptin, and fatty acid synthase inhibitors). Co-administration of a MAO-B inhibitor together with one or more other agents that are useful for treating the indications described above (e.g., obesity, diabetes, cardiometabolic disorders, and a combination thereof) is expected to be beneficial, by producing, for example, either additive or synergistic effects. Examples of additional agents include an appetite suppressant and a lipase inhibitor. Therefore, the present invention provides a method of treating obesity, diabetes, and/or cardiometabolic disorders, comprising administering a therapeutically effective amount of a compound of the present invention and a second component selected from an appetite suppressant (e.g., sibutramine, phentermine, fenfluramine, rimonabant, SLV319, BVT933, APD356, P57) and a gut lipase inhibitor (e.g., orlistat).

MAO-B inhibitors are expected to promote weight loss without appreciably reducing caloric intake. Co-administration of an MAO-B inhibitor together with an appetite suppressant is expected to produce either additive or synergistic effects on weight loss. Similarly, co-administration of an MAO-B inhibitor together with a lipase inhibitor is expected to produce either additive or synergistic effects on weight loss.

The ability of compounds to inhibit MAOs can be determined using the method of R. Uebelhack et al., Pharmacopsychiatry 31, 1988, p 187-192 (as described below).

Preparation of platelet-rich plasma and platelets. Venous blood from healthy subjects was collected between 8 and 8.30 a.m. after an overnight fast into EDTA-containing vacutainer tubes (11.6 mg EDTA /ml blood). After centrifugation of the blood at 250×g for 15minutes at 20° C., the supernatant platelet-rich plasma (PRP) was collected and the number of platelets in PRP counted with a cell counter (MOIAB, Hilden, Germany). 2 ml of PRP was spun at 1500×g for 10 min to yield a platelet pellet. The pellet was washed three times with ice-cold saline, resuspended in 2 ml Soerensen phoshate buffer, pH 7.4 and stored at −18° C. for one day.

MAO assay. Fresh PRP or frozen platelet suspension (100 μL) was generally preincubated for 10 min in the absence or presence of drugs at 37° C. in 100 μL of 0.9% NaCl solution or phosphate buffer pH 7.4, respectively, at 37° C. 50 μL of 2-phenylethylamine-[ethyl-1-14C]hydrochloride (PEA) solution (specific activity 56 Ci/mol, Amersham) was then added in a final concentration of 5 μM, and the incubation was continued for 30 min. The reaction was terminated by the addition of 50 μL of 4M HClO4. The reaction product of MAO, phenylacetaldehyde, was extracted into 2 mL of n-hexane. An aliquot of the organic phase was added to scintillator cocktail and the radioactivity was determined using a liquid scintillation counter. Product formation was linear with time for at least 60 min with appropriate platelet numbers. Blank values were obtained by including 2 mM pargyline in the incubation mixtures. All assays were performed in duplicate.

The ability of compounds to inhibit MAO activity can also be determined using the following method. cDNA's encoding human MAO-B can be transiently transfected into EBNA cells using the procedure described by E.-J. Schlaeger and K. Christensen (Transient Gene Expression in Mammalian Cells Grown in Serum-free Suspension Culture; Cytotechnology, 15: 1-13, 1998). After transfection, cells are homogeneized by means of a Polytron homogeneiser in 20 mM Tris HCl buffer, pH 8.0, containing 0.5 mM EGTA and 0.5 mM phenylmethanesulfonyl fluoride. Cell membranes are obtained by centrifugation at 45,000×g and, after two rinsing steps with 20 mM Tris HCl buffer, pH 8.0, containing 0.5 mM EGTA, membranes are eventually re-suspended in buffer and aliquots stored at −80° C. until use.

MAO-B enzymatic activity can be assayed using a spectrophotometric assay adapted from the method described by M. Zhou and N. Panchuk-Voloshina (A One-Step Fluorometric Method for the Continuous Measurement of Monoamine Oxidase Activity, Analytical Biochemistry, 253: 169-174, 1997). Briefly, membrane aliquots are incubated in 0. 1 M potassium phosphate buffer, pH 7.4, for 30 min at 37° C. with or without various concentrations of the compounds. After incubation, the enzymatic reaction is started by the addition of the MAO substrate tyramine together with 1 U/ml horse-radish peroxidase (Roche Biochemicals) and 80 μM N-acetyl-3,7,-dihydroxyphenoxazine (Amplex Red, Molecular Probes). The samples are further incubated for 30 min at 37° C. in a final volume of 200 μl and absorbance is determined at a wavelength of 570 nm using a SpectraMax plate reader (Molecular Devices). Background (non-specific) absorbance is determined in the presence of 10 μM L-deprenyl for MAO-B. IC₅₀ values are determined from inhibition curves obtained using nine inhibitor concentrations in duplicate, by fitting data to a four parameter logistic equation.

Compounds of the present invention are considered to be MAO-B inhibitors if they have an IC₅₀ value less than or equal to 10 μM. Additional examples of desirable activity levels of MAO-B inhibitors useful in the present invention include (a) an IC₅₀ value of 1 μM or lower, (b) an IC₅₀ value of 0.1 μM or lower, (c) an IC₅₀ value of 0.01 μM or lower, (d) an IC₅₀ value of 0.001 μM or lower, and (e) an IC₅₀ value of 0.0001 μM or lower.

In the present invention, MAO-B inhibitor(s) can be administered enterally, parenterally, orally, and transdermally. One skilled in this art is aware that the routes of administering the compounds of the present invention may vary significantly. In addition to other oral administrations, sustained release compositions may be favored. Other examples of routes include injections (e.g., intravenous, intramuscular, and intraperitoneal); subcutaneous; subdermal implants; buccal, sublingual, topical, rectal, vaginal, and intranasal administrations. Bioerodible, non-bioerodible, biodegradable, and non-biodegradable systems of administration may also be used. Another example of a route of administration is via a transdermal patch (e.g., twice daily, once daily, and once weekly). Transdermal patches, as is known in the art, can have a number of components to aid in drug delivery and a number layers (e.g., backing, adhesive drug layer, and release liner).

If a solid composition in the form of tablets is prepared, the main active ingredient can be mixed with a pharmaceutical vehicle, examples of which include silica, starch, lactose, magnesium stearate, and talc. The tablets can be coated with sucrose or another appropriate substance or they can be treated so as to have a sustained or delayed activity and so as to release a predetermined amount of active ingredient continuously. Gelatin capsules can be obtained by mixing the active ingredient with a diluent and incorporating the resulting mixture into soft or hard gelatin capsules. A syrup or elixir can contain the active ingredient in conjunction with a sweetener, which is preferably calorie-free, an antiseptic (e.g., methylparaben and/or propylparaben), a flavoring, and an appropriate color. Water-dispersible powders or granules can contain the active ingredient mixed with dispersants or wetting agents or with suspending agents such as polyvinylpyrrolidone, as well as with sweeteners or taste correctors. Rectal administration can be effected using suppositories, which are prepared with binders melting at the rectal temperature (e.g., cocoa butter and/or polyethylene glycols). Parenteral administration can be effected using aqueous suspensions, isotonic saline solutions, or injectable sterile solutions, which contain pharmacologically compatible dispersants and/or wetting agents (e.g., propylene glycol and/or polyethylene glycol). The active ingredient can also be formulated as microcapsules or microspheres, optionally with one or more carriers or additives. The active ingredient can also be presented in the form of a complex with a cyclodextrin, for example α-, β-, or γ-cyclodextrin, 2-hydroxypropyl-β-cyclodextrin, and/or methyl-β-cyclodextrin.

The dose of the MAO inhibitor administered daily will vary on an individual basis and to some extent may be determined by the severity of the disease being treated (e.g., obesity). The dose of the MAO inhibitor will also vary depending on the MAO inhibitor administered. Examples of dosages include about from 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 15, 20, 25, 30, 35, 40, 45, 50, 55, 60, 65, 70, 75, 80, 85, 90, 95, to 100 mg/kg of mammal body weight. If the MAO inhibitor administered is L-selegiline, then a dose of from approximately 0.5, 1, 2, 3, 4, 5, 6, 7, 8, 9, 10, 20, 30, 40, 50, 60, 70, 80, 90, to 100 mg/day may be administered. The MAO inhibitor can be administered in a single dose or in a number of smaller doses over a period of time; for example, a 15 mg/day dose of L-selegiline can be administered in three smaller 5 mg doses over the course of the day. The length of time during which the MAO inhibitor is administered varies on an individual basis, and can continue until the desired results are achieved (i.e., reduction of body fat, or prevention of a gain in body fat). Therapy could, therefore, last from 1 day to weeks, to months, to years depending upon the subject being treated, the desired results, and how quickly the subject responds to treatment in accordance with the present invention.

A possible example of a tablet of the present invention is as follows.

| Ingredient | mg/Tablet |
|---|---|
| Active ingredient | 100 |
| Powdered lactose | 95 |
| White corn starch | 35 |
| Polyvinylpyrrolidone | 8 |
| Na carboxymethylstarch | 10 |
| Magnesium stearate | 2 |
| Tablet weight | 250 |

A possible example of a capsule of the present invention is as follows.

| Ingredient | mg/Tablet |
|---|---|
| Active ingredient | 50 |
| Crystalline lactose | 60 |
| Microcrystalline cellulose | 34 |
| Talc | 5 |
| Magnesium stearate | 1 |
| Capsule fill weight | 150 |

In the above capsule, the active ingredient has a suitable particle size. The crystalline lactose and the microcrystalline cellulose are homogeneously mixed with one another, sieved, and thereafter the talc and magnesium stearate are admixed. The final mixture is filled into hard gelatin capsules of suitable size.

A possible example of an injection solution of the present invention is as follows.

| Ingredient | mg/Tablet |
|---|---|
| Active substance | 1.0 mg |
| 1 N HCl | 20.0 μl |
| acetic acid | 0.5 mg |
| NaCl | 8.0 mg |
| Phenol | 10.0 mg |
| 1 N NaOH | q.s. ad pH 5 |
| H₂O | q.s. ad 1 mL |

### EXAMPLES

The effect of MAO-B inhibitor in preventing the deposition of adiposity is illustrated in the following example. The unit of mg/kg refers to milligrams of substance administered per kilogram of mammal body weight.

### Example 1

Single-housed rats weighing 230-350 grams at the start of the experiment were provided free access to laboratory rodent chow pellets and tap water. Rats were divided into two groups, eight rats per group, matched for food intake and body weight over a two week baseline period. One group of rats was administered 10.0 mg/kg of L-selegiline hydrochloride orally by gavage once daily. The second group of rats (control rats) was similarly administered drug vehicle (0.25% methyl cellulose solution). Body weight and food intake were measured once a week for 14 weeks.

At the end of the experiment, rats were killed by exposure to carbon dioxide. Blood was collected by cardiac puncture, and rats were shaved and eviscerated. Retroperitoneal white adipose tissue, epidydimal white adipose tissue, adrenal gland, liver, kidney and testes were rapidly dissected and weighed. Plasma leptin levels were measured using a commercial radioimmunoassay kit.

Terminal carcass composition was measured. Shaved, eviscerated rats were coarsely ground and dried to a constant weight at 70° C. Dehydrated carcasses were finely ground in a blender. Lipid was extracted from a homogeneous sample with petroleum ether. Water and lipid contents were determined gravimetrically, and the remaining carcass component was termed fat free dry weight.

Compared to control rats, rats dosed with L-selegiline had a 14% lower weight gain over the course of the 14 week study (approximately 34 g less) than their untreated counterparts. Importantly, however, food intake was comparable between the two groups, indicating that the reduced weight gain was not a result of any CNS-mediated appetite-suppressant effects. Analysis of individual tissue and organ weights at the conclusion of the study revealed that the reduced weight gain was due almost exclusively to a selective reduction in fat tissue. Two distinct abdominal fat pads, epidydimal and retroperitoneal, weighed significantly less (23% and 27%, respectively) than corresponding fat pads removed from control rats. In sharp contrast, non-fat organs such as the adrenal gland, liver, kidney, and testes did not weigh less in the rats treated with L-selegiline.

Assessment of terminal whole carcass composition confirmed this finding. Compared to control rats, total body fat was reduced by 30% (or 32 grams) in rats dosed with L-selegiline. This accounts for 94% of the overall 34 gram weight loss. There was no reduction in whole carcass water content or lean body mass. Plasma leptin levels, a biomarker for overall adiposity, were significantly reduced (41%) in rats dosed with L-selegiline. Thus, the adipose tissue gain in the L-selegiline treated rats was 94% less than that of the untreated, control rats.

The invention has been described above in detail for the specific MAO inhibitor L-selegiline. It is understood that the invention is equally applicable to other MAO inhibitors. Accordingly, the foregoing description should not be read as pertaining only to the specific MAO inhibitor described, but rather should read consistent with and as support for the following claims that are to have their fullest fair scope.

What is claimed is:

1. A method for treating a disease, comprising: administering to a patient in need thereof a therapeutically effective amount of a MAO-B inhibitor or a pharmaceutically acceptable salt form thereof, wherein the disease is selected from obesity, diabetes, cardiometabolic disorders, and a combination thereof.

2. The method of claim 1, wherein the inhibitor is selected from: L-selegiline; desmethylselegiline; Rasagiline; Pargyline; Lazabemide; RO-16-6491, AGN 1135; MDL 72,974; MDL 72,145; MDL 72,638; LY 54761; MD 780236; Iproniazid; Phenelzine; Nialamide; Phenylhydrazine; 1-Phenylcyclopropylamine; Isocarboxazid; Tranylcypromine; and EVT 301, or a pharmaceutically acceptable salt thereof.

3. The method of claim 2, wherein the inhibitor is selected from: L-selegiline; Rasagiline; Lazabemide; and Pargyline, or a pharmaceutically acceptable salt thereof.

4. The method of claim 3, wherein the inhibitor is L-selegiline, or a pharmaceutically acceptable salt thereof.

5. The method of claim 3, wherein the inhibitor is Rasagiline, or a pharmaceutically acceptable salt thereof.

6. The method of claim 3, wherein the inhibitor is Lazabemide, or a pharmaceutically acceptable salt thereof

7. The method of claim 3, wherein the inhibitor is Pargyline, or a pharmaceutically acceptable salt thereof.

8. The method of claim 1, wherein the cardiometabolic disorder is selected from hypertension, dyslipidemias, high blood pressure, and insulin resistance.

9. The method of claim 1, wherein the inhibitor is of formula I or a pharmaceutically acceptable salt thereof: 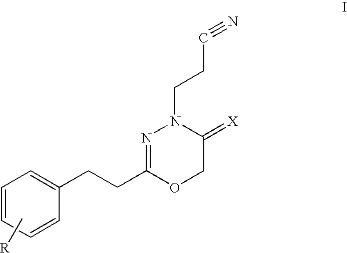
wherein:
X is O or S; and,
R is selected from H, or 3-Me, 4-Me, 3-Cl, 4-Cl, 3-OMe, 4-OMe, 3-NO₂, and 4-NO₂.

10. The method of claim 9, wherein:
X is O; and,
R is selected from H, 3-OMe, 4-OMe, 3-Me, and 3-Cl.

11. The method of claim 1, wherein the inhibitor is of formula II or IIa or apharmaceutically acceptable salt thereof: 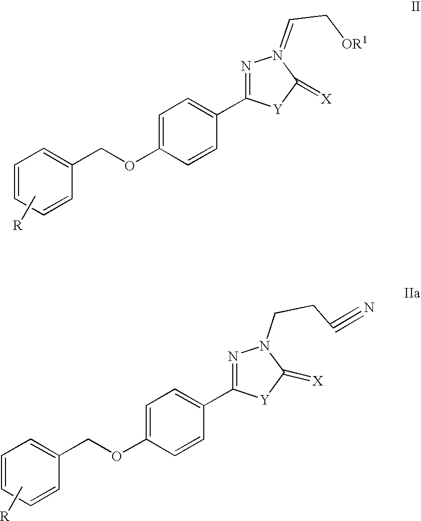
wherein:
X and Y are independently selected from O and S;
R is selected from H, CF₃, halogen, Me, NO₂, and OMe; and,
R¹ is selected from H and C₁₋₄ alkyl.

12. The method of claim 11, wherein:
X and Y are each O; and,
R is selected from H, 3-OMe, 4-OMe, 3-Me, 4-Me, 3-Cl, and 4-Cl.

13. The method of claim 11, wherein:
X is S;
Y is O; and,
R is H.

14. The method of claim 1, wherein the inhibitor is of formula III or a pharmaceutically acceptable salt thereof: 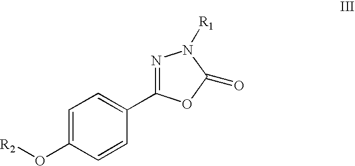
wherein:
R₁ is selected from H and a C₁₋₄ alkyl group, which is substituted with one or more groups selected from OH, phenoxy, C₁₋₄ alkoxy, C₁₋₄ alkylthio, SH, C₁₋₄ alkoxy-C₁₋₄ alkoxy, di-C₁₋₄ alkylamino, N-C₁₋₄ alkyl-N-propynylamino, and a C₃₋₄ alkynyl; and,
R₂ is a C₁₋₈ alkyl group, which is substituted with one or more groups selected from halogen, OH, 1-imidazolyl, 3-tetrahydropyranyl, and trifluoro-C₃₋₅-alkenyl.

15. The method of claim 14, wherein the compound is selected from:
5-[4-(4,4,4-trifluorobutoxy)phenyl]-3-methoxyethyl-1,3,4-oxadiazol-2(3H)-one;
5-[4-(4,4,4-trifluorobutoxy)phenyl]-3-hydroxyethyl-1,3,4-oxadiazol-2(3H)-one;
5-[4-(4,4,4-trifluorobutoxy)phenyl]-3-methylthioethyl-1,3,4-oxadiazol-2(3H )-one;
5-[4-(4,4,4-trifluoro-2-butenyloxy)phenyl]-3-methoxyethyl-1,3,4-oxadiazol-2(3H)-one;
5-[4-(4,4,4-trifluoro-3(R)-hydroxybutoxy)phenyl]-3-methoxyethyl-1,3,4-oxadiazol-2(3H) -one; and,
5-[4-(tetrahydropyran-3-ylmethoxy)phenyl]-3-methoxyethyl-1,3,4-oxadiazol-2(3H)-one, or pharmaceutically acceptable salts thereof.

16. The method of claim 1, wherein the inhibitor is of formula IV or a pharmaceutically acceptable salt thereof: 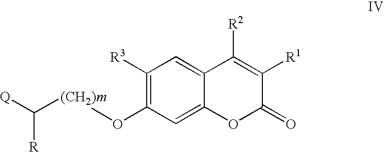
wherein:
Q is phenyl substituted with 0-3 groups selected from halogen, C₁₋₆ alkyl, and C₁₋₆ alkoxy;
alternatively, Q is phenyl substituted with one group selected from NO₂, —CN, and trifluoromethyl;
alternatively, Q is selected from a naphthyl ring and 5-10 membered heteroaryl consisting of carbon atoms and 1-3 heteroatoms selected from O, N, and S(O)ₚ, wherein the naphthyl and heteroaryl are substituted with 0-2 groups selected from halogen, C1-6 alkyl, C₁₋₆ alkoxy, C₁₋₄ alkoxy-C₁₋₄ alkylene, C₃₋₈ cycloalkyl, C₁₋₄ alkyl-C₃₋₈ cycloalkylene, benzyl, and 5-6 membered heteroaryl consisting of carbon atoms and 1-3 heteroatoms selected from O, N, and S(O)ₚ,
R is selected from H and C₁₋₆ alkyl;
R¹ is selected from H, halogen, and C₁₋₆ alkyl;
R² is selected from H, halogen, and C₁₋₆ alkyl;
R³ is selected from H, halogen, and C₁₋₆ alkyl;
m is selected from 0, 1, 2, 3, and 4; and,
p is selected from 0, 1, and 2.

17. The method of claim 16, wherein: 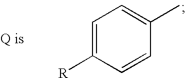
R is selected from H, Me, and isopropyl;
alternatively, Q is selected from the following:

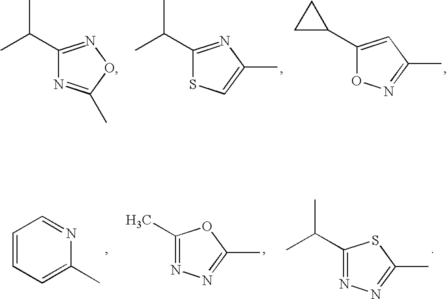

18. The method of claim 16, wherein the inhibitor is selected from:
3,4-Dimethyl-7-(4-isopropylphenyl)-methoxycoumarin;
3,4-Dimethyl-7-(2-naphthyl)-methoxycoumarin;
7-(4-tert-Butylphenyl)-methoxy-3,4-dimethylcoumarin;
3,4-Dimethyl-7-(2-methylphenyl)-methoxycoumarin;
3,4-Dimethyl-7-(3 -methylphenyl)-methoxycoumarin;
3,4-Dimethyl-7-(4-methylphenyl)-methoxycoumarin;
3,4-Dimethyl-7-(2,5 -dimethylphenyl)-methoxycoumarin;
7-(4-Methoxyphenyl)-methoxy-3,4-dimethylcoumarin;
7-(4-Trifluoromethylphenyl)-methoxy-3,4-dimethylcoumarin;
7-(3-Trifluoromethylphenyl)-methoxy-3,4-dimethylcoumarin;
6-Ethyl-3,4-dimethyl-7-(2-phenyl)-ethoxycoumarin;
7-(4-Isopropylphenyl)-methoxycoumarin;
4-Methyl-7-(4-isopropylphenyl)-methoxycoumarin;
3-Methyl-7-(4-isopropylphenyl)-methoxycoumarin;
3-Chloro-4-methyl-7-(4-isopropylphenyl)-methoxycoumarin;
7-(3-Phenylpropoxy)-coumarin;
3,4-Dimethyl-7-(3 -phenylpropoxy)-coumarin;
6-Chloro-3,4-dimethyl-7-(4-isopropylphenyl)-methoxycoumarin;
7-(2-Benzylthiazol-4-yl)-methoxy-3,4-dimethylcoumarin;
7-(2-Isopropylthiazol-4-yl)-methoxy-3,4-dimethylcoumarin;
7-(3-Cyclopentylisoxazol-5-yl)-methoxy-3,4-dimethylcoumarin; 7-[3-(1-Methoxyethyl)-isoxazol-5-yl]-methoxy-3,4-dimethylcoumarin;
7-(2-Cyclopropylthiazol-4-yl)-methoxy-3,4-dimethylcoumarin;
6-Ethyl-7-(5-isopropyl-1-methylpyrazol-3-yl)-methoxy-3,4-dimethylcoumarin;
6-Ethyl-3,4-dimethyl-7-(2-methyl-1,3,4-thiadiazol-5-yl)-methoxycoumarin;
7-(3-Cyclohexylisoxazol-5-yl)-methoxy-3,4-dimethylcoumarin;
7-(2-tert-Butylthiophen-5-yl)-methoxy-3,4-dimethylcoumarin;
3,4-Dimethyl-7-(2-cyclopropyl-1,3,4-thiadiazol-5-yl)-methoxycoumarin;
3,4-Dimethyl-7-[3-(1-methylcyclopropyl)-isoxazol-5-yl]-methoxycoumarin;
3,4-Dimethyl-7-[3-(tetrahydrofuran-3-yl)-isoxazol-5-yl]-methoxycoumarin;
3,4-Dimethyl-7-(3-cyclopentylisoxazol-5-yl)-methoxycoumarin;
3,4-Dimethyl-7-(3-cyclohexylisoxazol-5-yl)-methoxycoumarin;
6-Chloro-3,4-dimethyl-7-(2-pyridinyl)-methoxycoumarin;
3,6-Dichloro-4-methyl-7-(5-methyl-1,3,4-thiadiazol-2-yl)-methoxycoumarin; and,
3,6-Dichloro-4-methyl-7-(2-isopropylthiazol-4-yl)-methoxycoumarin,
or pharmaceutically acceptable salts thereof.

19. The method of claim 1, wherein the inhibitor is of formula V and VI or a pharmaceutically acceptable salt thereof: 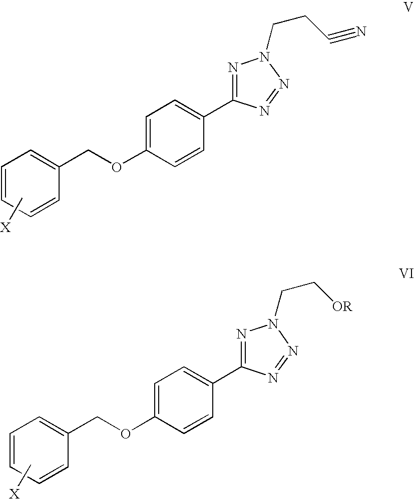
wherein:
X is selected from H, halogen, Me, OMe, CF₃, and phenyl; and,
R is selected from H and C₁₋₄ alkyl.

20. The method of claim 19, wherein X is selected from H, 2-Cl, 3-Cl, 4-Cl, 2-F, 3-F, 4-F, 2-Me, 3-Me, 4-Me, 2-CF₃, 3-CF₃, and 4-CF₃.

21. The methed of claim 1, wherein the inibitor is of formula VII or a pharmaceutically acceptable salt thereof: 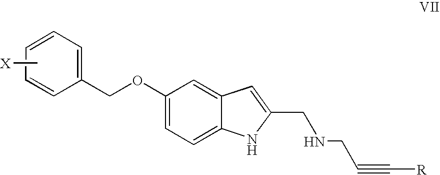
wherein:
R is selected from H and Me; and,
X is selected from H 2-Cl, 3-Cl, 4-Cl, 2F, 3-F, 4-F, 2-Me, 3-Me, 4-Me, 2-CF₃, 3-CF₃, and 4-CF₃.

22. The method of claim 1, wherein the inhibitor is of formula VIII, IX, or X, or a pharmaceutically acceptable salt thereof: 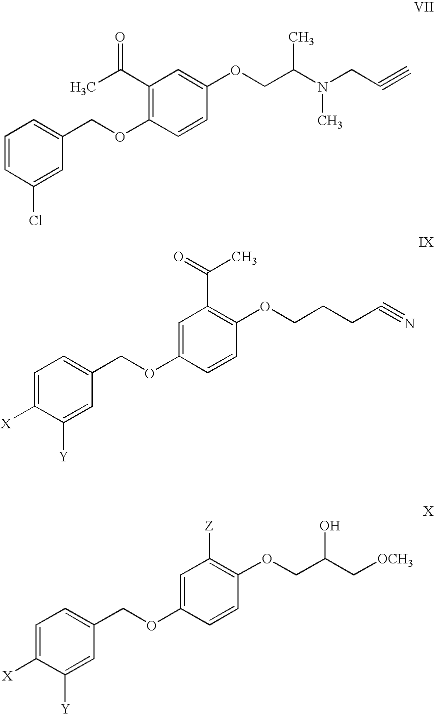
wherein:
X and Y are selected from H, Cl, F, CH₃ and CF₃; and,
Z is selected from —COCH₃ and CHO.

23. The method of claim 1, wherein the inhibitor is of formula XI or a pharmaceutically acceptable salt thereof: 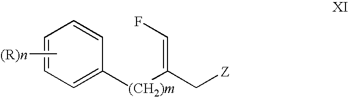
wherein:
R is selected from C₁₋₈ alkyl, C₁₋₈ alkoxy, OH, halogen, CF₃, NO₂, C₁₋₆ alkylcarbonyl, benzoyl, phenyl, 1-naphthyl, 2-naphthyl, 1-indenyl, 2-indenyl, 3-indenyl, 1-fluorenyl, 2-fluorenyl, 9-fluorenyl, 1-piperdinyl, 2-piperdinyl, 3-piperidinyl, 2-pyrrolyl, 3-pyrrolyl; 2-thienyl, 3-thienyl, 2-furanyl, 3-furanyl, 2-indolyl, 3-indolyl, 2-thianaphthenyl, 3-thianaphthenyl, 2-benzofuranyl, and 3-benzofuranyl;
Z is NH₂ or OH;
m is selected from 0, 1, 2, and 3; and,
n is selected from 0, 1, 2, and 3.

24. The method of claim 23, wherein the inhibitor is selected from:
(Z)-or (E)-(p-fluorophenethyl)-3-fluoroallylamine,
(Z)-or (E)-2-(2′-methoxy)phenyl-3-fluoroallylamine,
(Z)-or (E)-2-(3′-methoxy)phenyl-3-fluoroallylamine,
(Z)-or (E)-2-(4′-methoxy)phenyl-3-fluoroallylamine,
(Z)-or (E)-2-(3′-hydroxy)phenyl-3-fluoroallylamine,
(Z)-or (E)-N-ethyl2-(3′-methoxy)phenyl-3-fluoroallylamine,
(Z)-or (E)-2-(3′,4′-dimethoxy)phenyl-3-fluoroallylamine,
(Z)-or (E)-N-ethyl2-(3′,4′-dimethoxy)phenyl-3-fluoroallylamine,
(Z)-or (E)-2-(4′-chloro)phenyl-3-fluoroallylamine,
(Z)-or (E)-2-(3′-trifluoromethyl)phenyl-3-fluoroallylamine.
(Z)-or (E)-2-(.alpha.-naphthyl)-3-fluoroallylamine,
(Z)-or (E)-2-(.beta.-naphthyl)-3-fluoroallylamine,
(E)-2-(2′-methoxy)phenyl-3-fluoroallyl alcohol,
(E)-2-(3′-methoxy)phenyl-3-fluoroallyl alcohol,
(E)-2-(4′-methoxy)phenyl-3-fluoroallyl alcohol,
(E)-2-(3′,4′-dimethoxy)phenyl-3-fluoroallyl alcohol,
(E)-2-(2′-methoxy)benzyl-3-fluoroallyl alcohol,
(E)-2-(3′-methoxy)benzyl-3-fluoroallyl alcohol,
(E)-2-(4′-methoxy)benzyl-3-fluoroallyl alcohol,
(E)-2-phenyl-3-fluoroallyl alcohol,
(E)-2-benzyl-3-fluoroallyl alcohol,
(E)-2-(3′,4′-dimethoxy)benzyl-3-fluoroallyl alcohol,
Z)-or (E)-2-(3′-methoxyphenyl)-3-fluoroallylamine, and
(Z)-or (E)-2-(3′,4′-dimethoxyphenyl)-3-fluoroallylamine;
or pharmaceutically acceptable salts thereof.

25. The method of claim 1, wherein the inhibitor is of formula XII or a pharmaceutically acceptable salt thereof: 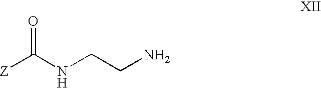
wherein:
Z is aryl or is selected from a 5-or 6-membered heteroaryl shown below: 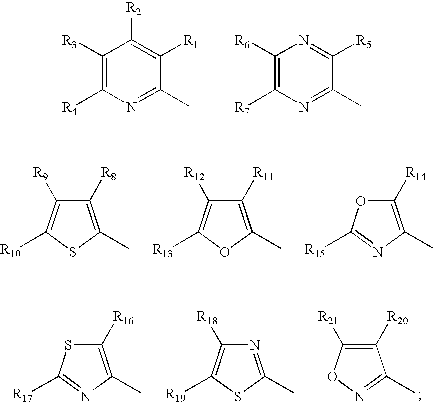
at least two of R₁, R₂, R₃, and R₄ are H and the remaining two are independently selected from H, halogen, NO₂, NH₂, OH, C₁₋₆ alkoxy, C₁₋₆ alkyl, phenyloxy, and phenylmethyloxy, the phenyl group of phenyloxy and phenylmethyloxy being optionally substituted with a group selected from halogen, C₁₋₆ alkyl, C₁₋₆ alkoxy, NO₂, and OH;
R₅, R₆, and R₇ are independently selected from H and halogen;
R₈, R₉ and R₁₀ are independently selected from H, halogen, and C₁₋₆ alkyl, provided that at least one of R₈, R₉, and R₁₀ is other than H;
R₁₁, R₁₂, and R₁₃ are independently selected from H and halogen, provided that at least one of R₁₁, R₁₂ and R₁₃ are other than H;
R₁₄, R₁₅, R₁₆, R₁₈ and R₁₉ are selected from H, halogen, and C₁₋₆ alkyl;
R₁₇ is selected from H and halogen; and,
R₂₀ and R₂₁ are selected from H and C₁₋₆ alkyl.

26. The method of claim 25, wherein the inhibitor is selected from:
N-(2-aminoethyl)-4-methoxypyridine-2-carboxamide,
N-(2-aminoethyl)thiazole-2-carboxamide,
N-(2-aminoethyl)-4-bromopyridine-2-carboxamide,
N-(2-aminoethyl)-4-chloropyridine-2-carboxamide,
N-(2-aminoethyl)-2-chlorothiazole-4-carboxamide,
N-(2-aminoethyl)-5-methylisoxazole-3-carboxamide,
N-(2-aminoethyl)-6-bromopyridine-2-carboxamide,
N-(2-aminoethyl)-6-chloropyridine-2-carboxamide,
N-(2-aminoethyl)-5-bromothiazole-4-carboxamide,
N-(2-aminoethyl)-3-aminopyridine-2-carboxamide,
N-(2-aminoethyl)pyridine-2-carboxamide,
N-(2-aminoethyl)-5-chloropyridine-2-carboxamide,
N-(2-aminoethyl)-2-chlorothiazole-4-carboxamide hydrochloride,
N-(2-aminoethyl)-3-aminopyridine-2-carboxamide dihydrochloride,
N-(2-aminoethyl)pyridine-2-carboxamide dihydrochloride, and
N-(2-aminoethyl)-5-chloropyridine-2-carboxamide hydrochloride,
or pharmaceutically acceptable salts thereof.

27. The method of claim 1, wherein the inhibitor is of formula XIII or a pharmaceutically acceptable salt thereof: 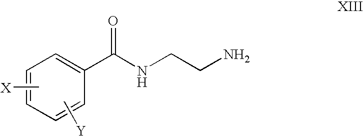
wherein:
X and Y are independently selected from H, Cl, F, Br, C₁₋₆ alkyl, C₁₋₆ alkoxy, CF₃, —CN, sulfamoyl, mono(C₁₋₆ alkyl)sulfamoyl, and di(C₁₋₆ alkyl)sulfamoyl;
provided that Y is other than H when X is 3-Br;
alternatively, X and Y, when on adjacent carbon atoms, together form a methylenedioxy group.

28. The method of claim 27, wherein the inhibitor is selected from:
N-(2-aminoethyl)-p-chlorobenzamide,
N-(2-aminoethyl)-p-fluorobenzamide,
N-(2-aminoethyl)-p-bromobenzamide,
N-(2-aminoethyl)-3,4-dichlorobenzamide, and
N-(2-aminoethyl)-2,4-dichlorobenzamide,
or pharmaceutically acceptable salts thereof.

29. The method of claim 1, wherein the inhibitor is of formula XIV or a pharmaceutically acceptable salt thereof: 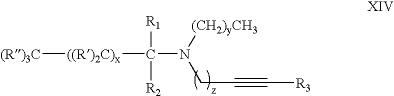
wherein: 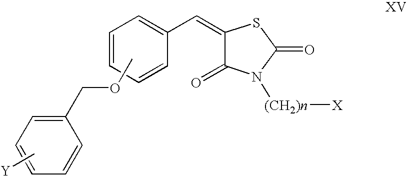
R₁ is selected from H and C₁₋₆ alkyl;
R₂ is selected from H and C₁₋₆ alkyl;
R₃ is selected from H and C₁₋₆ alkyl;
R′ is selected from H and halogen;
R″ is selected from H and halogen;
x is selected from 1, 2, 7, 8, 9, 10, 11, 12, and 13;
y is selected from 0, 1, 2, 3, 4, and 5; and,
z is selected from 1, 2, 3, 4, and 5.

30. The method of claim 29, wherein the inhibitor is selected from:
N-(2-propyl)-N-methylpropargylamine-HCl;
N-(2-butyl)-N-methylpropargylamine-HCl;
N-(1-butyl)-N-methylpropargylamine-HCl;
N-(2-heptyl)-N-methylpropargylamine-HCl;
N-(1-heptyl)-N-methylpropargylamine-HCl;
N-(2-pentyl)-N-methylpropargylamine-HCl;
N-(1-pentyl)-N-methylpropargylamine-oxalate;
N-(2-decyl)-N-methylpropargylamine-HCl;
N-(2-dodecyl)-N-methylpropargylamine-HCl; and,
R(-)-N-(2-butyl)-N-methylpropargylamine-oxalate,
or pharmaceutically acceptable salts thereof.

31. The method of claim 1, wherein the inhibitor is of formula XV or a pharmaceutically acceptable salt thereof: t,0800
wherein:
X is —CN or —SCN;
Y is selected from H, halogen, C₁₋₆ alkyl, C₁₋₆ alkoxy, and CF₃; and,
n is selected from 1, 2, 3, 4, 5, and 6.

32. The method of claim 31, wherein the inhibitor is selected from:
2,4-dioxo-5-[3-(phenylmethoxy)-phenylmethylene]-4-thiazolidinebutanenitrile, and 2,4-dioxo-5-[3-(phenylmethoxy)-phenylmethylene]-4-thiazolidinepentanenitrile,
or pharmaceutically acceptable salts thereof.

33. The method of claim 1, wherein the inhibitor is of formula XVI or a pharmaceutically acceptable salt thereof: 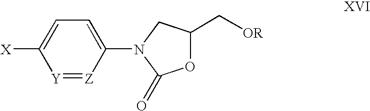
wherein:
R is selected from H and C₁₋₆ alkyl;
X is selected from C₂₋₈ cycloalkenyl, bicyclo[2.2.1]hept-2-yl optionally substituted by phenyl-2-oxo-5-methoxymethyloxazolidinyl; bicyclo[2.2.1]-hept-5-en-2-yl; adamantyl; C₃₋₆ cycloalkyl; and piperidinyl;
X is optionally mono-or multiply-substituted by halogen, NH₂, C₁₋₆ alkyl, —CN, O, hydroxyimino, ethylenedioxy, —OR₁, ═CR₂R₃, —(CH₂)ₙR₄, —COR₅, and —NR₆R₇;
R₂ is selected from H and C₁₋₆ alkyl;
R₃ is selected from H, —CN, C₁₋₆ alkyl, phenyl, and CO₂—C₁₋₆ alkyl;
R₄ is selected from —CN, NH₂, —NHCOCH₃, —C(O)C₆H₄-halogen, phenyl, and OH;
R₅ is selected from C₁₋₆ alkyl, —CH═CHC₆H₅, —C₆H₄—CF₃, —OC(CH₃)₃, and C₁₋₆ alkoxy;
R₆ is selected from H and COCH₃;
R₇ is selected from COCH₃, benzyl, and —(CH₂)ₙNHCOC₆H₄-halogen; and,
n is selected from 1, 2, and 3.

34. The method of claim 33, wherein the inhibitor is selected from:
(RS)-3-(4-Cyclohexyl-phenyl)-5-hydroxymethyl-oxazolidin-2-one;
(RS)-3-(4-cyclohexylophenyl)-5-methoxymethyl-oxazolidin-2-one;
(R)-3-(4-cyclohexyl-phenyl)-5-methoxymethyl-oxazolidin-2-one;
(RS)-3-[4-(4-oxocyclohexyl)-phenyl]-5-methoxymethyl-oxazolidin-2-one;
(RS)-3-[4-(trans-4-hydroxy-cyclohexyl)-phenyl]-5-methoxymethyl-oxazolidin-2-one;
(RS)-3-[4-(4-hydroxy-imino-cyclohexyl)-phenyl]-5-methoxymethyl-oxazolidin-2-one;
(R)-3-[4-trans-4-hydroxy-cyclohexyl)-phenyl]-5-methoxymethyl-oxazolidin-2-one;
(RS)-3-[4-(trans-4-methoxy-cyclohexyl)-phenyl]-5-methoxymethyl-oxazolidin-2-one;
(R)-3-[4-(4-oxo-cyclohexyl)-phenyl]-5-methoxymethyl-oxazolidin-2-one;
(RS)-3-[trans-4-[4-(5-methoxymethyl-2-oxo-oxazolidin-3-yl)-phenyl]-cyclohexyloxy]-propionitrile;
(RS)-trans-4-[4-(5-methoxymethyl-2-oxo-oxazolidin-3-yl)-phenyl]-cyclohexyl ester;
(RS)-3-[4-(cis-or-trans-4-hydroxymethyl-cyclohexyl)-phenyl]-5-methoxymethyl-oxazolidin-2-one;
(RS)-3-[4-(cis-or trans-4-hydroxy-4-methyl-cyclohexyl)-phenyl]-5-methoxymethyl-oxazolidin-2-one.
(RS)-[trans-4-[4-(5-methoxymethyl-2-oxo-oxazolidin-3-yl)-phenyl]-cyclohexyl]-acetonitrile;
(R)-[trans-4-[4-(5-methoxymethyl-2-oxo-oxazolidin-3-yl)-phenyl]-cyclohexyl]-acetonitrile;
(RS)-4-[4-(5-methoxymethyl-2-oxo-oxazolidin-3-yl)-phenyl]-1-(1-oxo-3-phenyl-2-(E)-propenyl)-piperidine;
(RS)-3-(trans-4-[4-(5-methoxymethyl-2-oxo-oxazolidin-3-yl)-phenyl]-cyclohexyloxy]-ethanamine;
(R)-[trans-4-[4-(5-methoxymethyl-2-oxo-oxazolidin-3-yl)-phenyl]-cyclohexyloxy]-acetonitrile;
(RS)-trans-4-[4-(5-methoxymethyl-2-oxo-oxazolidin-3-yl)-phenyl]-cyclohexyloxy]-acetonitrile; and,
(R)-3-[4-[trans-4-(3-amino-propoxy)-cyclohexyl]-phenyl]-5-methoxymethyl-oxazolidin-2-one;
or pharmaceutically acceptable salts thereof.

35. The method of claim 1, wherein the inhibitor is of formula XVII or stereoisomers or a pharmaceutically acceptable salt thereof: 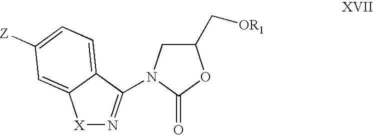
wherein:
X is selected from O, S, and NR;
R₁ is selected from H and C₁₋₄ alkyl;
Z is selected from H, Me, OR₃, —CH═CH—R₄, and —CH₂CH₂R₄;
R₃ is selected from H and a benzyl group, which is optionally substituted by a group selected from halogen, NO₂, —OCH₂O—, CH₃OC₂CH₂—, butyl, 4,4,4-trifluorobutyl, 4,4,4-trifluoro-3-hydroxybutyl, and 4,4,4-trifluorobut-2-enyl group; and,
R₄ is selected from phenyl, 3,3,3-trifluoropropyl, and 3,3,3-trifluoro-2-hydroxypropyl.

36. The method of claim 35, wherein the inhibitor is selected from:
(S)-5-Methoxymethyl-3->6-(4,4,4-trifluorobutoxy)-1,2-benzisoxazol-3-yl!oxazolidin-2-one
or a pharmaceutically acceptable salt thereof.

37. The method of claim 1, wherein the inhibitor is of formula XVIII or a pharmaceutically acceptable salt thereof: 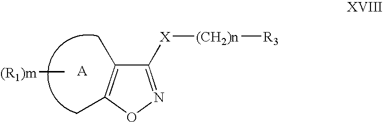
wherein:
R₁ is selected from H; halogen; C₁₋₆ alkyl; C₁₋₄ alkyl substituted by a halogen atom or a C₁₋₄ alkoxy; C₁₋₆ alkoxy; halogeno-C₁₋₆ alkoxy; OH; C₁₋₆ alkylthio; NH₂; C₁₋₆—NH; (C₁₋₆ alkyl)₂N; C₁₋₆ alkanoyl; C₁₋₆ alkanoyl-NH; C₁₋₆ alkanoyloxy; C₁₋₆ alkoxy-carbonyl; CO₂H; (C₁₋₆ alkylthio)thiocarbonyl; carbamoyl; mono-C₁₋₆ alkylcarbamoyl; di-C₁₋₆ alkylcarbamoyl; NO₂; and, —CN;
R₃ is NH₂;
m is 1;
n is selected from 1, 2, 3, 4, 5, and 6;
Ring A is a phenyl ring fused with the isoxazole ring or a naphthyl ring fused with the isoxazole ring; and,
X is selected from O and S.

38. The method of claim 37, wherein the inhibitor is selected from:
3-(2-aminoethoxy)-1,2-benzisoxazole,
3-(2-aminoethoxy)-5-fluoro-1,2-benzisoxazole,
3-(2-aminoethylthio)-5-fluoro-1,2-benzisoxazole,
3-(2-aminoethoxy)-5-chloro-1,2-benzisoxazole,
3-(2-aminoethylthio)-5-chloro-1,2-benzisoxazole,
3-(2-aminoethoxy)-6-chloro-1,2-benzisoxazole,
3-(2-aminoethoxy)-7-chloro-1,2-benzisoxazole,
3-(2-aminoethoxy)-5-bromo-1,2-benzisoxazole,
3-(2-aminoethoxy)-5-methyl-1,2-benzisoxazole,
3-(2-aminoethoxy)-5-methyl-1,2-benzisoxazole,
3-(2-aminoethoxy)-6-methoxy-1,2-benzisoxazole,
3-(2-aminoethoxy)-7-methyl-1,2-benzisoxazole,
3-(2-aminoethoxy)-5-methoxy-1,2-benzisoxazole,
3-(2-aminoethylthio)-5-methoxy-1,2-benzisoxazole,
3-(2-aminoethoxy)-5-difluoromethoxy-1,2-benzisoxazole,
3-(2-aminoethoxy)-5-methoxycarbonyl-1,2-benzisoxazole,
3-(2-aminoethoxy)-5-nitro-1,2-benzisoxazole,
3-(2-aminoethylthio)-5-nitro-1,2-benzisoxazole,
3-(2-aminoethoxy)-4-cyano-1,2-benzisoxazole,
3-(2-aminoethoxy)-4-carbamoyl-1,2-benzisoxazole hydrochloride,
3-(2-aminoethylthio)-5-amino-1,2-benzisoxazole dihydrochloride,
3-(2-aminoethoxy)-1,2-naphtho[2,3-e]isoxazole hydrochloride,
3-(2-aminoethoxy)-5-methylamino-1,2-benzisoxazole dihydrochloride,
3-(2-aminoethoxy)-5-dimethylamino-1,2-benzisoxazole dihydrochloride,
3-(2-aminoethoxy)-7-carboxy-1,2-benzisoxazole hydrochloride,
3-(2-aminoethoxy)-5-hydroxy-1,2-benzisoxazole hydrochloride, and
3-(2-aminoethoxy)-5-acetoxy-1,2-benzisoxazole hydrochloride,
or pharmaceutically acceptable salts thereof.

39. The method of claim 1, wherein the inhibitor is of formula XIX or a pharmaceutically acceptable salt thereof: 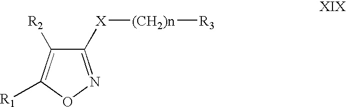
wherein:
X is selected from O and S;
R₁ is selected from C₆₋₁₄.aryl substituted with 0-3 substituents or a 5-6-membered aromatic heterocyclic group substituted with 0-3 substituents and consisting of carbon atoms and 1-2 heteroatoms independently selected from nitrogen, oxygen and sulfur atoms;
the substituents for R₁ are independently selected from halogen; C₁₋₆ alkyl; C₁₋₆ alkyl substituted with a halogen or a C₁₋₆ alkoxy; C₁₋₆ alkoxy; C₆₋₁₄ aryl, C₇₋₁₈ aralkyl; C₆₋₁₄ aryloxy; and C₇₋₁₈ aralkyloxy, wherein the aralkyloxy group is substituted with 0-3 substituents independently selected from halogen; C₁₋₆ alkyl; C₁₋₆ alkoxy; —CN; NO₂; OH; C₁₋₇ alkanoyl; C₁₋₇ alkanoyloxy; C₂₋₇ alkoxycarbonyl; NH₂; a carbamoyl; a mono(C₁₋₆ alkyl)carbamoyl; a di(C₁₋₆ alkyl)carbamoyl, and a mono C₇₋₁₅ arylcarbonylamino substituted with 0-3 substituents selected from a halogen, C₁₋₆ alkyl, and C₁₋₆ alkoxy;
R₂ is selected from H; halogen; C₁₋₆ alkyl substituted with a halogen or C₁₋₆ alkoxy; C₂₋₆ alkenyl; C₂₋₆ alkynyl; C₃₋₁₀ cycloalkyl; C₃₋₁₀ cycloalkenyl; C₁₋₆ alkoxy; —CN; CO₂H; C₁₋₇ alkanoyl; C₂₋₇ alkoxycarbonyl; carbamoyl; mono-C₁₋₆ alkyl-carbamoyl; and, di-C₁₋₆ alkyl-carbamoyl;
R₃ is selected from NH₂; C₁₋₆ alkyl-NH; (C₁₋₆ alkyl)₂N; C₁₋₇ alkanoylNH; C₂₋₇ alkoxycarbonyl-NH; C₇₋₁₅ arylcarbonyl-NH-substituted with 0-3 substituents independently selected from halogen, C₁₋₆ alkyl, and C₁₋₆ alkoxy; and a 5-6-membered saturated heterocyclic group (attached through a ring nitrogen atom), which consists of carbon atoms, one nitrogen atom, and an additional nitrogen or oxygen atom; and,
n is selected from 2, 3, 4, 5, and 6.

40. The method of claim 39, wherein the inhibitor is selected from:
3-(2-aminoethoxy)-5-phenylisoxazole,
3-(2-aminoethoxy)-4-chloro-5-phenylisoxazole,
3-(2-aminoethoxy)-4-ethyl-5-phenylisoxazole,
3-(2-aminoethoxy)-5-phenyl-4-propylisoxazole,
3-(2-aminoethoxy)-4-isopropyl-5-phenylisoxazole,
3-(2-aminoethoxy)-4-isobutyl-5-phenylisoxazole,
3-(2-aminoethoxy)-5-(2-chlorophenyl)-4-isopropylisoxazole,
3-(2-aminoethoxy)-5-(4-chlorophenyl)isoxazole,
3-(2-aminoethoxy)-5-(4-chlorophenyl)-4-isopropylisoxazole,
3-(2-aminoethoxy)-5-(2,4-dichlorophenyl)-4-isopropylisoxazole,
3-(2-aminoethoxy)-5-(2-furyl)-4-isopropylisoxazole,
3-(2-aminoethoxy)-5-(2-thienyl)isoxazole,
3-(2-aminoethoxy)-4-chloro-5-(2-thienyl)isoxazole,
3-(2-aminoethoxy)-4-isopropyl-5-(2-thienyl)isoxazole, and
4-allyl-3-(2-aminoethoxy)-5-phenylisoxazole,
and pharmaceutically acceptable salts thereof.

41. The method of claim 1, wherein the inhibitor is of formula XX or a pharmaceutically acceptable salt thereof: 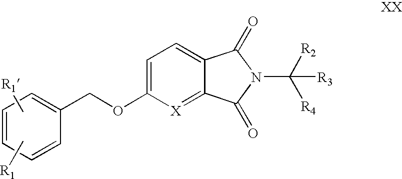
wherein:
X is selected from N and CH;
R₁ and R₁′ are independently selected from H, halogen, C₁₋₆ alkyl, halo C_{1 6} alkyl, —CN, C₁-6 alkoxy, C₁₋₆ haloalkoxy, and CF₃;
R₂ is selected from H, (CH₂)ₙCN, (CH₂)ₚOR₄, (CH₂)ₙCON(R₄)₂, (CH₂)ₙCO₂R₄, CHR₅(CH₂)ₙOR₆, (CH₂)ₙN(R₄)₂, (CH₂)ₙNHCOR₄, and (CH₂)ₙNHCOOR₄;
R₃ is selected from H, alkyl, (CH₂)ₙO—C₁₋₆ alkyl, (CH₂)S—C₁₋₆ alkyl, (CH₂)ₙS(O)—C₁₋₆ alkyl, benzyl, and —CN;
R₄ is independently selected from H and alkyl;
R₅ is selected from H, C₁₋₆ alkyl, —CN, and CONH₂; and,
R6 is selected from H and C₁₋₆ alkyl.

42. The method of claim 41, wherein the inhibitor is selected from:
2-[5-(4-fluoro-benzyloxy)-1,3-dioxo-1,3-dihydro-isoindol-2-yl]-acetamide,
(S)-2-[5-(4-fluoro-benzyloxy)-1,3-dioxo-1,3-dihydro-isoindol-2-yl]-propionamide,
(S)-2-[5-(4-fluoro-benzyloxy)-1,3-dioxo-1,3-dihydro-isoindol-2-yl]-3-hydroxy-propionamide,
(R)-2-[5-(4-fluoro-benzyloxy)-1,3-dioxo-1,3-dihydro-isoindol-2-yl]-propionamide,
2-[5-(3-fluoro-benzyloxy)-1,3-dioxo-1,3-dihydro-isoindol-2-yl]-propionamide,
2-[5-(3-fluoro-benzyloxy)-1,3-dioxo-1,3-dihydro-isoindol-2-yl]-acetamide,
2-[5-(3-fluoro-benzyloxy)-1,3-dioxo-1,3-dihydro-isoindol-2-yl]-3-hydroxy-propionamide,
N-{2-[5-(4-fluoro-benzyloxy)-1,3-dioxo-1,3-dihydro-isoindol-2-yl]-ethyl}-acetamide,
2-(2-amino-ethyl)-5-(4-fluoro-benzyloxy)-isoindole-1,3-dione,
5-(4-fluoro-benzyloxy)-2-piperidin-4-yl-isoindole-1,3-dione,
5-(4-fluoro-benzyloxy)-2-(2-hydroxy-ethyl)-isoindole-1,3-dione,
5-(4-fluoro-benzyloxy)-2-(2-methoxy-ethyl)-isoindole-1,3-dione,
5-(3-fluoro-benzyloxy)-2-(2-methoxy-ethyl)-isoindole-1,3-dione,
(S)-5-(4-fluoro-benzyloxy)-2-(2-methoxy-1-methyl-ethyl)-isoindole-1,3-dione,
(S)-5-(3-fluoro-benzyloxy)-2-(2-methoxy-1-methyl-ethyl)-isoindole-1,3-dione,
(S)-5-(2-fluoro-benzyloxy)-2-(2-methoxy-1-methyl-ethyl)-isoindole-1,3-dione,
(S)-2-(2-methoxy-1-methyl-ethyl)-5-(4-trifluoromethyl-benzyloxy)-isoindole-1,3-dione,
(S)-5-(4-bromo-benzyloxy)-2-(2-methoxy-1-methyl-ethyl)-isoindole-1,3-dione,
(S)-5-(3,4-difluoro-benzyloxy)-2-(2-methoxy-1-methyl-ethyl)-isoindole-1,3-dione,
5-(3-fluoro-benzyloxy)-2-(2-hydroxy-ethyl)-isoindole-1,3-dione,
5-(4-fluoro-benzyloxy)-2-(3,3,3-trifluoro-2-hydroxy-propyl)-isoindole-1,3-dione,
5-(3,5-bis-trifluoromethyl-benzyloxy)-2-(2-methoxy-1-methyl-ethyl)-isoindole-1,3-dione,
2-(2-ethylsulfanyl-ethyl)-5-(4-fluoro-benzyloxy)-isoindole-1,3-dione,
(S)-2-[5-(4-fluoro-benzyloxy)-1,3-dioxo-1,3-dihydro-isoindol-2-yl]-thiopropionamide,
2-(2-ethylsulfanyl-ethyl)-5-(3-fluoro-benzyloxy)-isoindole-1,3-dione,
5-(4-fluoro-benzyloxy)-1,3-dioxo-1,3-dihydro-isoindol-2-yl]-acetonitrile, and
[5-(3-fluoro-benzyloxy)-1,3-dioxo-1,3-dihydro-isoindol-2-yl]-acetonitrile,
or pharmaceutically acceptable salts thereof.

43. The method of claim 1, wherein the inhibitor is of formula XXI or a pharmaceutically acceptable salt thereof: 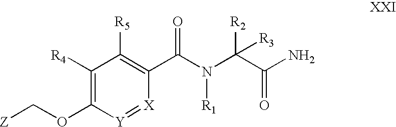
wherein:
X and Y are independently selected from N and CR₆;
Z is selected from C₁₋₆-haloalkyl, aryl, aryl substituted by one or more substituents selected from C₁₋₆ alkyl, halogen, C₁₋₆ haloalkyl, C₁₋₆ alkoxy, and —CN;
R₁ is selected from H and C₁₋₆ alkyl;
R₂ is selected from H and C₁₋₆ alkyl;
R₃ is selected from H and C₁₋₆ alkyl;
R₄ is selected from H and C₁₋₆ alkyl;
R₅ is selected from H and C₁₋₆ alkyl; and,
R₆ is selected from H and C₁₋₆ alkyl.

44. The method of claim 43, wherein the inhibitor is selected from:
5-(3-fluoro-benzyloxy)-pyridine-2-carboxylic acid carbamoylmethyl-amide,
5-(4-fluoro-benzyloxy)-pyridine-2-carboxylic acid carbamoylmethyl-amide,
5-(3,4-difluoro-benzyloxy)-pyridine-2-carboxylic acid carbamoylmethyl-amide,
(S)-5-(3-fluoro-benzyloxy)-pyridine-2-carboxylic acid (1-carbamoyl-ethyl)-amide,
(S)-5-(4-fluoro-benzyloxy)-pyridine-2-carboxylic acid (1-carbamoyl-ethyl)-amide,
(S)-5-(3,4-difluoro-benzyloxy)-pyridine-2-carboxylic acid (1-carbamoyl-ethyl)-amide,
6-Benzyloxy-N-carbamoylmethyl-nicotinamide,
N-Carbamoylmethyl-6-(3-fluoro-benzyloxy)-nicotinamide,
N-Carbamoylmethyl-6-(4-fluoro-benzyloxy)-nicotinamide,
(S)-6-Benzyloxy-N-(1-carbamoyl-ethyl)-nicotinamide,
(S)-N-(1-Carbamoyl-ethyl)-6-(3-fluoro-benzyloxy)-nicotinamide, and
(S)-N-(1-Carbamoyl-ethyl)-6-(4-fluoro-benzyloxy)-nicotinamide,
or pharmaceutically acceptable salts thereof.

45. The method of claim 1, wherein the inhibitor is of formula XXII or a pharmaceutically acceptable salt thereof: 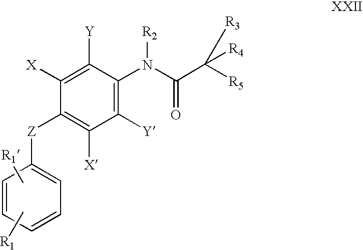
wherein:
R₁ and R₁′ are independently selected from H, halogen, C₁₋₆ alkyl, C₁₋₆ haloalkyl, —CN, C₁₋₆ alkoxy, C₁₋₆ haloalkoxy, CF₃, OH, and CHO;
X and Y are independently selected from H, halogen, C₁₋₆ alkyl, C₁₋₆ haloalkyl, —CN, C₁₋₆ alkoxy, C₁₋₆ haloalkoxy, and CF₃;
X′ and Y′ are independently selected from H, halogen, C₁₋₆ alkyl, C₁₋₆ haloalkyl, —CN, C₁₋₆ alkoxy, C₁₋₆ haloalkoxy, and CF₃;
R₂ is selected from H and C₁₋₆ alkyl;
R₃ and R₄ are independently selected from H, C₁₋₆ alkyl, C₁₋₆ alkoxy, and —CO₂—C₁₋₆ alkyl;
alternatively, R₃ and R4, together with the C-atom to which they are attached, form a C₁₋₇-cycloalkyl ring;
R₅ is selected from CONR₆R₇, CO₂—C₁₋₆ alkyl, —CN, N(R)₂, and NHC(O)R;
R₆ and R₇ are independently selected from H, C₁₋₆ alkyl, NH₂, and OH;
R is H or C₁₋₆ alkyl;
Z is selected from —CHRO—, —OCHR—, —CH₂S—, —SCH₂—, —CH₂CH₂—, —CH═CH—, and —C═C—; and,
n is selected from 0, 1, 2, and 3.

46. The method of claim 45, wherein the inhibitor is selected from:
N-[4-(3-fluoro-benzyloxy)-phenyl]-malonamic acid methyl ester;
N-[3-fluoro-4-(3-fluoro-benzyloxy)-phenyl]-malonamic acid methyl ester;
N-[4-(4-fluoro-benzyloxy)-phenyl]-malonamic acid methyl ester;
N-[2-fluoro-4-(3-fluoro-benzyloxy)-phenyl]-malonamic acid methyl ester;
N-[4-(2,4-difluoro-benzyloxy)-phenyl]-malonamic acid methyl ester;
N-[4-(2-fluoro-benzyloxy)-phenyl]-malonamic acid methyl ester;
N-[4-(2,4,5-trifluoro-benzyloxy)-phenyl]-malonamic acid methyl ester;
N-[2-fluoro-4-(4-fluoro-benzyloxy)-phenyl]-malonamic acid methyl ester;
N-[4-(3,5-bis-trifluoromethyl-benzyloxy)-2-fluoro-phenyl]-malonamic acid methyl ester;
N-[4-(3-fluoro-benzyloxy)-3-methyl-phenyl]-malonamic acid methyl ester;
N-[3-chloro-4-(3-fluoro-benzyloxy)-phenyl]-malonamic acid methyl ester;
cyclopropane-1,1-dicarboxylic acid amide [4-(3-fluoro-benzyloxy)-phenyl]-amide;
N-[4-(3-fluoro-benzyloxy)-phenyl]-malonamide;
N-[4-(3-fluoro-benzyloxy)-phenyl]-2-methyl-malonamide;
N-[3-fluoro-4-(3-fluoro-benzyloxy)-phenyl]-malonamide;
N-[4-(4-fluoro-benzyloxy)-phenyl]-malonamide;
N-[4-(2,4-difluoro-benzyloxy)-phenyl]-malonamide;
N-[4-(2,4,5-trifluoro-benzyloxy)-phenyl]-malonamide;
N-[4-(2-fluoro-benzyloxy)-phenyl]-malonamide;
N-(4-benzyloxy-phenyl)-malonamide;
N-[4-(4-chloro-benzyloxy)-phenyl]-malonamide;
N-[4-(3-fluoro-benzyloxy)-2-hydroxy-phenyl]-malonamide;
N-[2-fluoro-4-(4-fluoro-benzyloxy)-phenyl]-malonamide;
N-[4-(3-fluoro-benzyloxy)-3-methyl-phenyl]-malonamide;
N-[3-chloro-4-(3-fluoro-benzyloxy)-phenyl]-malonamide;
cyclopropane-1,1-dicarboxylic acid amide [2-fluoro-4-(4-fluoro-benzyloxy)-phenyl]-amide;
2-Acetylamino-N-[2-fluoro-4-(4-fluoro-benzyloxy)-phenyl]-acetamide;
2-Acetylamino-N-[2-fluoro-4-(3-fluoro-benzyloxy)-phenyl]-acetamide;
N-[2-Fluoro-4-(4-fluoro-benzyloxy)-phenyl]-2-formylamino-acetamide;
N-[2-Fluoro-4-(3-fluoro-benzyloxy)-phenyl]-2-formylamino-acetamide;
11) 2-amino-N-[2-fluoro-4-(4-fluoro-benzyloxy)-phenyl]-acetamide;
14) N-{4-[2-(4-fluoro-phenyl)-vinyl]-phenyl}-malonamic acid methyl ester;
N-{4-[2-(3-fluoro-phenyl)-vinyl]-phenyl}-malonamide;
N-{4-[2-(4-fluoro-phenyl)-vinyl]-phenyl}-malonamide;
N-{4-[2-(3-fluoro-phenyl)-vinyl]-phenyl}-malonamic acid methyl ester.
N-[4-(3-Fluoro-benzyloxy)-phenyl]-2-methyl-malonamic acid methyl ester;
N-[4-(3-Fluoro-benzyloxy)-phenyl]-2-methoxy-malonamic acid methyl ester;
N-[4-(3-Fluoro-benzyloxy)-2-trifluoromethyl-phenyl]-malonamic acid methyl ester;
N-[4-(3-Fluoro-benzyloxy)-phenyl]-N-methyl-malonamic acid methyl ester;
N-[4-(4-Trifluoromethyl-benzyloxy)-phenyl]-malonamic acid methyl ester;
N-(4-Benzyloxy-phenyl)-malonamic acid methyl ester;
N-[2-Fluoro-4-(4-trifluoromethyl-benzyloxy)-phenyl]-malonamic acid methyl ester;
N-[2-Fluoro-4-(4-fluoro-benzyloxy)-phenyl]-malonamic acid ethyl ester;
N-[4-(3-Fluoro-benzyloxy)-3-formyl-phenyl]-malonamic acid methyl ester;
N-[4-(3-Fluoro-benzyloxy)-3-methoxy-phenyl]-malonamic acid methyl ester; and
N-[2-Fluoro-4-(4-fluoro-benzyloxy)-phenyl]-2,2-dimethyl-malonamic acid methyl ester.
N-[4-(3-Fluoro-phenoxymethyl)-phenyl]-malonamic acid methyl ester;
N-[4-(3-Fluoro-benzylsulfanyl)-phenyl]-malonamic acid methyl ester;
2-[4-(3-Fluoro-benzyloxy)-phenylcarbamoyl]-malonic acid dimethyl ester;
N-{4-[2-(4-Fluoro-phenyl)-vinyl]-phenyl}-malonamic acid methyl ester;
N-{4-[2-(3-Fluoro-phenyl)-vinyl]-phenyl}-malonamic acid methyl ester;
N-{4-[2-(4-Fluoro-phenyl)-ethyl]-phenyl}-malonamic acid methyl ester;
N-{4-[2-(3-Fluoro-phenyl)-ethyl]-phenyl}-malonamic acid methyl ester;
N-{4-[2-(4-Methoxy-phenyl)-vinyl]-phenyl}-malonamic acid methyl ester; N-{4-[2-(4-Chloro-phenyl)-ethyl]-phenyl}-malonamic acid methyl ester.
N-[4-(3-Fluoro-benzyloxy)-phenyl]-2,2-dimethyl-malonamide;
N-[4-(4-Trifluoromethyl-benzyloxy)-phenyl]-malonamide;
N-[2-Fluoro-4-(3-fluoro-benzyloxy)-phenyl]-malonamide;
N-[2,5-Difluoro-4-(3-fluoro-benzyloxy)-phenyl]-malonamide;
N-[4-(3-Fluoro-benzyloxy)-phenyl]-N′-hydroxy-malonamide;
N-[4-(3,5-Bis-trifluoromethyl-benzyloxy)-2-fluoro-phenyl]-malonamide;
N-[2-Fluoro-4-(4-trifluoromethyl-benzyloxy)-phenyl]-malonamide;
N-[4-(3-Fluoro-benzyloxy)-3-methoxy-phenyl]-malonamide;
N-[4-(3-Fluoro-benzylsulfanyl)-phenyl]-malonamide;
N-{4-[1-(3-Fluoro-phenyl)-ethoxy]-phenyl}-malonamide;
N-[4-(3-Fluoro-phenoxymethyl)-phenyl]-malonamide;
2-Ethyl-N-[4-(3-fluoro-benzyloxy)-phenyl]-malonamide;
N-{4-[2-(4-Fluoro-phenyl)-vinyl]-phenyl}-malonamide;
N-{4-[2-(3-Fluoro-phenyl)-vinyl]-phenyl}-malonamide;
N-{4-[2-(4-Fluoro-phenyl)-ethyl]-phenyl}-malonamide;
N-{4-[2-(4-Chloro-phenyl)-ethyl]-phenyl}-malonamide.
2-Cyano-N-[4-(3-fluoro-benzyloxy)-phenyl]-acetamide;
N-[4-(3-Fluoro-benzyloxy)-phenyl]-2-hydrazinocarbonyl-acetamide;
Cyclopropane-1,1-dicarboxylic acid amide [4-(3-fluoro-phenoxymethyl)-phenyl]-amide;
2-Amino-N-[2-fluoro-4-(4-fluoro-benzyloxy)-phenyl]-acetamide (1:1) hydrochloride;
(R)-2-Amino-N-[2-fluoro-4-(4-fluoro-benzyloxy)-phenyl]-propionamide;
2-Amino-N-[2-fluoro-4-(4-fluoro-benzyloxy)-phenyl]-propionamide;
1-[2-Fluoro-4-(4-fluoro-benzyloxy)-phenylcarbamoyl]-2S-methyl-propyl-ammonium chloride;
(R)-2-Acetylamino-N-[2-fluoro-4-(4-fluoro-benzyloxy)-phenyl]-propionamide;
(R)-N-[2-Fluoro-4-(4-fluoro-benzyloxy)-phenyl]-2-formylamino-propionamide;
2-Amino-N-[2-fluoro-4-(3-fluoro-benzyloxy)-phenyl]-acetamide hydrochloride (1:1);
2-Amino-N-[2-fluoro-4-(4-trifluoromethyl-benzyloxy)-phenyl]-acetamide (1:1) hydrochloride;
2-Amino-N-[4-(3,5-bis-trifluoromethyl-benzyloxy)-2-fluoro-phenyl]-acetamide (1:1) hydrochloride;
2-Acetylamino-N-[4-(3,5-bis-trifluoromethyl-benzyloxy)-2-fluoro-phenyl]-acetamide; and,
2-Amino-N-[4-(3-fluoro-benzyloxy)-phenyl]-acetamide hydrochloride;
or pharmaceutically acceptable salts thereof.

47. The method of claim 1, wherein the inhibitor is of formulas XXIII, XXIV, and XXV, or a pharmaceutically acceptable salt thereof: 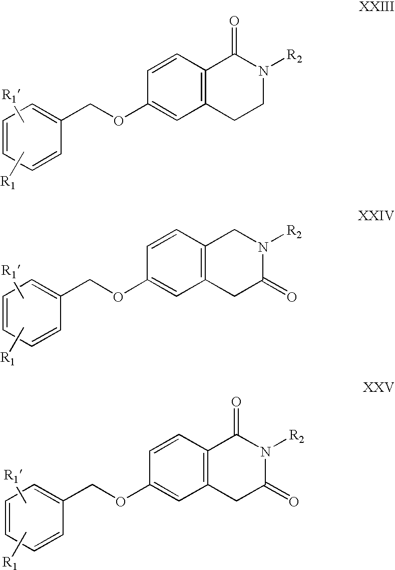
wherein:
R₁ is selected from H, halogen, C₁₋₆ alkyl, C₁₋₆ haloalkyl, —CN, C₁₋₆ alkoxy, C₁₋₆ haloalkoxy, and CF₃;
R₁′ is selected from H, halogen, C₁₋₆ alkyl, C₁₋₆ haloalkyl, —CN, C₁₋₆ alkoxy, C₁₋₆ haloalkoxy, and CF₃;
R₂ is selected from H and CR₃R₄R₅;
R₃ is selected from (CH₂)ₙCO—NR₆R₇, (CH₂)ₙ,CN, (CH₂)ₚOR₈, (CH₂)ₙNR₆R₇, (CH₂)ₙNHCOR₉, (CH₂)ₚSR₈, and (CH₂)ₚSOR₉;
R₄ is selected from H, C₁₋₆ alkyl, (CH₂)ₚOR₈, (CH₂)ₚSR₈, and benzyl;
R₅ is selected from H, C₁₋₆ alkyl, (CH₂)ₚOR₈, (CH₂)ₚSR₈, and benzyl;
R₆ and R₇ are independently selected from H and C₁₋₆ alkyl;
R₈ is selected from H and C₁₋₆ alkyl;
R₉ is C₁₋₆ alkyl;
n is selected from 0, 1, and 2; and,
p is selected from 1 and 2.

48. The method of claim 47, wherein the inhibitor is selected from:
2-[6-(3-fluoro-benzyloxy)-1-oxo-3,4-dihydro-1H-isoquinolin-2-yl]-acetamide,
2-[6-(3-fluoro-benzyloxy)-1-oxo-3,4-dihydro-1H-isoquinolin-2-yl]-propionamide
2-[6-(4-fluoro-benzyloxy)-1-oxo-3,4-dihydro-1H-isoquinolin-2-yl]-propionamide,
2-[6-(3,4-difluoro-benzyloxy)-1-oxo-3,4-dihydro-1H-isoquinolin-2-yl]-propionamide,
2-[6-(3-fluoro-benzyloxy)-1-oxo-3,4-dihydro-1H-isoquinolin-2-yl]-propionamide;
2-(R)-[6-(3-fluoro-benzyloxy)-1-oxo-3,4-dihydro-1H-isoquinolin-2-yl]-propionamide,
2-(R)-[6-(4-fluoro-benzyloxy)-1-oxo-3,4-dihydro-1H-isoquinolin-2-yl]-propionamide,
2-(S)-[6-(4-fluoro-benzyloxy)-1-oxo-3,4-dihydro-1H-isoquinolin-2-yl]-propionamide,
2-(S)-[6-(4-fluoro-benzyloxy)-1-oxo-3,4-dihydro-1H-isoquinolin-2-yl]-3-hydroxy-propionamide,
2-(R)-[6-(2,6-difluoro-benzyloxy)-1-oxo-3,4-dihydro-1H-isoquinolin-2-yl]-propionamide;
2-[6-(3-fluoro-benzyloxy)3,4-dihydro-1H-isoquinolin-2-yl]-propionamide,
2-[6-(4-fluoro-benzyloxy)3,4-dihydro-1H-isoquinolin-2-yl]-acetamide,
2-[6-(3-fluoro-benzyloxy)-3,4-dihydro-1H-isoquinolin-2-yl]-acetamide,
2-[6-(4-fluoro-benzyloxy)3,4-dihydro-1H-isoquinolin-2-yl]-propionamide;
2-(R)-[6-(4-fluoro-benzyloxy)-1,3-dioxo-3,4-dihydro-1H-isoquinolin-2-yl]-propionamide,
2-(S)-[6-(4-fluoro-benzyloxy)-1,3-dioxo-3,4-dihydro-1H-isoquinolin-2-yl]-propionamide;
2-(S)-[6 (4-fluoro-benzyloxy)-3-oxo-3,4-dihydro-1H-isoquinolin-2-yl]-propionamide,
2(R)-[6-(4-fluoro-benzyloxy)-3-oxo-3,4-dihydro-1H-isoquinolin-2-yl]-propionamide
2-[1-oxo-6-(4-trifluoromethyl-benzyloxy)-3,4-dihydro-1H-isoquinolin-2-yl]-propionamide;
2-[6-(2-Fluoro-benzyloxy)-1-oxo-3,4-dihydro-1H-isoquinolin-2-yl]-propionamide;
2(R)-[6-(2,6-Difluoro-benzyloxy)-1-oxo-3,4-dihydro-1H-isoquinolin-2-yl]-propionamide;
2(R)-[6-(2-Fluoro-benzyloxy)-1-oxo-3,4-dihydro-1H-isoquinolin-2-yl]-propionamide;
2(R) [6-(2,3-Difluoro-benzyloxy)-1-oxo-3,4-dihydro-1H-isoquinolin-2-yl]-propionamide;
2(R) [6-(3-Fluoro-benzyloxy)-1-oxo-3,4-dihydro-1H-isoquinolin-2-yl]-propionamide;
2(S)-[6-(3-Fluoro-benzyloxy)-1-oxo-3,4-dihydro-1H-isoquinolin-2-yl]-propionamide; and
2(S)-[6-(3,4-Difluoro-benzyloxy)-1-oxo-3,4-dihydro-1H-isoquinolin-2-yl]-propionamide;
6-(3-Fluoro-benzyloxy)-3,4-dihydro-2H-isoquinolin-1-one;
2-[6-(4-Fluoro-benzyloxy)-1-oxo-3,4-dihydro-1H-isoquinolin-2-yl]-acetamide;
2(R)-[6-(3-Cyano-benzyloxy)-1-oxo-3,4-dihydro-1H-isoquinolin-2-yl]-propionamide
2-[1-oxo-6-(4-trifluoromethyl-benzyloxy)-3,4-dihydro-1H-isoquinolin-2-yl]-propionamide;
2-[6-(2-Fluoro-benzyloxy)-1-oxo-3,4-dihydro-1H-isoquinolin-2-yl]-propionamide;
2(R)-[6-(2,6-Difluoro-benzyloxy)-1-oxo-3,4-dihydro-1H-isoquinolin-2-yl]-propionamide;
2(R)-[6-(2-Fluoro-benzyloxy)-1-oxo-3,4-dihydro-1H-isoquinolin-2-yl]-propionamide;
2(R) [6-(2,3-Difluoro-benzyloxy)-1-oxo-3,4-dihydro-1H-isoquinolin-2-yl]-propionamide;
2(R) [6-(3-Fluoro-benzyloxy)-1-oxo-3,4-dihydro-1H-isoquinolin-2-yl]-propionamide;
2(S)-[6-(3-Fluoro-benzyloxy)-1-oxo-3,4-dihydro-1H-isoquinolin-2-yl]-propionamide;
2(S)-[6-(3,4-Difluoro-benzyloxy)-1-oxo-3,4-dihydro-1H-isoquinolin-2-yl]-propionamide;
6-(3-Fluoro-benzyloxy)-3,4-dihydro-2H-isoquinolin-1-one;
2-[6-(4-Fluoro-benzyloxy)-1-oxo-3,4-dihydro-1H-isoquinolin-2-yl]-acetamide;
2(R)-[6-(3-Cyano-benzyloxy)-1-oxo-3,4-dihydro-1H-isoquinolin-2-yl]-propionamide
2(R)-[6-(3,5-Difluoro-benzyloxy)-1-oxo-3,4-dihydro-1H-isoquinolin-2-yl]-propionamide;
3-[6-(4-Fluoro-benzyloxy)-3,4-dihydro-1H-isoquinolin-2-yl]-propionamide;
3-[6-(3-Fluoro-benzyloxy)-3,4-dihydro-1H-isoquinolin-2-yl]-propionamide;
2(R)-[(3,4-Difluoro-benzyloxy)-1-oxo-3,4-dihydro-1H-isoquinolin-2-yl]-propionamide;
2-[6-(3-Chloro-benzyloxy)-1-oxo-3,4-dihydro-1H-isoquinolin-2-yl]-propionamide;
3-[6-(3-Fluoro-benzyloxy)-1-oxo-3,4-dihydro-1H-isoquinolin-2-yl]-propionamide;
2-(S)-[6-(4-Fluoro-benzyloxy)-1-oxo-3,4-dihydro-1H-isoquinolin-2-yl]-4-methylsulfanyl -butyramide;
2-(R)-[6-(4-Fluoro-benzyloxy)-1-oxo-3,4-dihydro-1H-isoquinolin-2-yl]-3-hydroxy-propionamide;
2-(S)-[6-(4-Fluoro-benzyloxy)-1-oxo-3,4-dihydro-1H-isoquinolin-2-yl]-4-methyl-pentanoic acid amide;
2-(S)-[6-(4-Fluoro-benzyloxy)-1-oxo-3,4-dihydro-1H-isoquinolin-2-yl]-butyramide;
2-(R)-[6-(4-Fluoro-benzyloxy)-1-oxo-3,4-dihydro-H-isoquinolin-2-yl]-3-phenyl-propionamide;
2-(S)-[6-(4-Fluoro-benzyloxy)-1-oxo-3,4-dihydro-1H-isoquinolin-2-yl]-3-methyl-butyramide;
2-(S)-[6-(4-Fluoro-benzyloxy)-1-oxo-3,4-dihydro-1H-isoquinolin-2-yl]-3-phenyl-propionamide;
2-[6-(4-Fluoro-benzyloxy)-1-oxo-3,4-dihydro-1H-isoquinolin-2-yl]-isobutyramide;
6-(3-Fluoro-benzyloxy)-2-(2-hydroxy-1-methyl-ethyl)-3,4-dihydro-2H-isoquinolin-1-one;
6-(3-Fluoro-benzyloxy)-2-(2-hydroxy-ethyl)-3,4-dihydro-2H-isoquinolin-1-one;
6-(3-Fluoro-benzyloxy)-2-(2-methoxy-1-methyl-ethyl)-3,4-dihydro-2H-isoquinolin-1-one;
6-(3-Fluoro-benzyloxy)-2-(2-methoxy-ethyl)-3,4-dihydro-2H-isoquinolin-1-one;
2-(2-Ethoxy-ethyl)-6-(3-fluoro-benzyloxy)-1,2,3,4-tetrahydro-isoquinoline;
6-(4-Fluoro-benzyloxy)-2-(2-methoxy-ethyl)-1,2,3,4-tetrahydro-isoquinoline;
2-(2-Ethoxy-ethyl)-6-(4-fluoro-benzyloxy)-1,2,3,4-tetrahydro-isoquinoline;
[6-(3-Fluoro-benzyloxy)-1-oxo-3,4-dihydro-1H-isoquinolin-2-yl]-acetonitrile;
3 -[6-(3-Fluoro-benzyloxy)-1-oxo-3,4-dihydro-1H-isoquinolin-2-yl]-propionitrile;
6-(4-Fluoro-benzyloxy)-2-(4,4,4-trifluoro-butyl)-1,2,3,4-tetrahydro-isoquinoline;
6-(4-Fluoro-benzyloxy)-2-(tetrahydro-furan-2-ylmethyl)-1,2,3,4-tetrahydro-isoquinoline;
2-[6 (4-Fluoro-benzyloxy)-1-oxo-3,4-dihydro-1H-isoquinolin-2-yl]-propionamide;
2-(R)-[6-(3-Fluoro-benzyloxy)-1-oxy-3,4-dihydro-1H-isoquinolin-2-yl]-propionamide;
2(R)-[6-(4-Fluoro-benzyloxy)-1-oxo-3,4-dihydro-1H-isoquinolin-2-yl]-propionamide; and,
2(S)-[6-(4-Fluoro-benzyloxy)-1-oxo-3,4-dihydro-1H-isoquinolin-2-yl]-propionamide,
or pharmaceutically acceptable salts thereof.

49. The method of claim 1, wherein the inhibitor is of of formula XXVI and XXVII, or a pharmaceutically acceptable salt thereof: 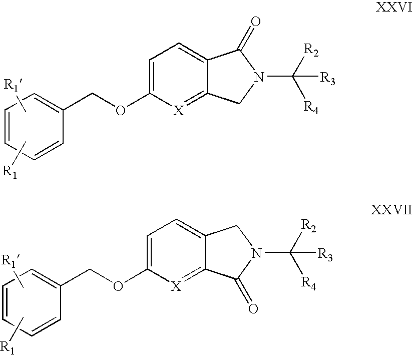
wherein:
X is selected from N and CH;
R₁ and R₁′ are independently selected from H, halogen, C₁₋₆ alkyl, C₁₋₆ haloalkyl, —CN, C₁₋₆ alkoxy, C₁₋₆ haloalkoxy, and CF₃;
R₂ is selected from H, (CH₂)ₙCN, (CH₂)ₙOR₄, (CH₂)ₙCON(R₄)₂, (CH₂)ₙCO₂R₄, CHR₅(CH₂)ₙOR₆, (CH₂)ₙ-isoindole-1,3-dionyl, and (CH₂)ₙN(R₄)₂;
R₃ is selected from H, C₁₋₆ alkyl, (CH₂)ₙO—C₁₋₆ alkyl, (CH₂)ₙS—C₁₋₆ alkyl, (CH₂)ₙS(O)—C₁₋₆ alkyl, and benzyl;
R₄ is selected from H and C₁₋₆ alkyl;
R₅ is selected from H, C₁₋₆ alkyl, —CN, and CONH₂;
R₆ is selected from H and C₁₋₆ alkyl; and,
n is selected from 0, 1, and 2.

50. The method of claim 49, wherein the inhibitor is selected from:
2-[5-(3-fluoro-benzyloxy)-1-oxo-1,3-dihydro-isoindol-2-yl]-acetamide,
2-[5-(3-fluoro-benzyloxy)-1-oxo-1,3-dihydro-isoindol-2-yl]-propionamide,
(S)-2-[6-(3-fluoro-benzyloxy)-1-oxo-1,3-dihydro-isoindol-2-yl]-propionamide,
(R)-2-[6-(3-fluoro-benzyloxy)-1-oxo-1,3-dihydro-isoindol-2-yl]-propionamide,
2-[5-(4-fluoro-benzyloxy)-1-oxo-1,3-dihydro-isoindol-2-yl]-acetamide,
2-[1-oxo-5-(4-trifluoromethyl-benzyloxy)-1,3-dihydro-isoindol-2-yl]-acetamide,
5-(3-Fluoro-benzyloxy)-2-(2-methoxy-ethyl)-2,3-dihydro-isoindol-1-one,
2-[6-(3-fluoro-benzyloxy)-1-oxo-1,3-dihydro-isoindol-2-yl]-acetamide,
(R)-2-[6-(3-fluoro-benzyloxy)-1-oxo-1,3-dihydro-isoindol-2-yl]-propionamide,
(S)-2-[1-oxo-6-(4-trifluoromethyl-benzyloxy)-1,3-dihydro-isoindol-2-yl]-propionamide,
(R)-2-[1-oxo-6-(4-trifluoromethyl-benzyloxy)-1,3-dihydro-isoindol-2-yl]-propionamide,
[6-(3-fluoro-benzyloxy)-1-oxo-1,3-dihydro-isoindol-2-yl]-acetic acid methyl ester,
[1-oxo-6-(4-trifluoromethyl-benzyloxy)-1,3-dihydro-isoindol-2-yl]-acetic acid methyl ester,
2-(2-Methoxy-ethyl)-6-(3-fluoro-benzyloxy)-2,3-dihydro-isoindol-1-one,
2-(2-methoxy-ethyl)-6-(4-trifluoromethyl-benzyloxy)-2,3-dihydro-isoindol-1-one,
2-(2-amino-ethyl)-6-(4-trifluoromethyl-benzyloxy)-2,3-dihydro-isoindol-1-one 1:1 hydrochloride, and
2-(2-amino-ethyl)-6-(4-trifluoromethyl-benzyloxy)-2,3-dihydro-isoindol-1-one 1:1 hydrochloride,
or pharmaceutically acceptable salts thereof.

51. The method of claim 1, wherein the inhibitor is of formula XXVIII and XXIX, or a pharmaceutically acceptable salt thereof: 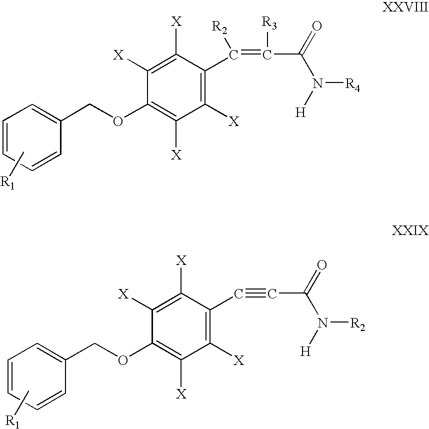
wherein:
X is selected from H and F;
R₁ is selected from H, halogen, C₁₋₆ alkyl, C₁₋₆ haloalkyl, —CN, C₁₋₆ alkoxy, and C₁₋₆ haloalkoxy;
R₂ is selected from H and C₁₋₆ alkyl;
R₃ is selected from H and C₁₋₆ alkyl; and,
R₄ is selected from H and C₁₋₆ alkyl.

52. The method of claim 51, wherein the inhibitor is selected from:
N-methyl-3-[4-(4-methyl-benzyloxy)-phenyl]-acrylamide;
3-[4-(3-methoxy-benzyloxy)-phenyl]-N-methyl-acrylamide;
3-[4-(3-fluoro-benzyloxy)-phenyl]-2,N-dimethyl-acrylamide;
3-[4-(3-fluoro-benzyloxy)-phenyl]-N-methyl-acrylamide;
N-methyl-3-[4-(4-trifluoromethyl-benzyloxy)-phenyl]-acrylamide;
3-[4-(3,4-difluoro-benzyloxy)-phenyl]-N-methyl-acrylamide;
3-[4-(4-fluoro-benzyloxy)-phenyl]-N-methyl-acrylamide;
3-[4-(3-fluoro-benzyloxy)-phenyl]-2,N-dimethyl-propionamide;
3-[4-(3,4-difluoro-benzyloxy)-phenyl]-propionamide;
3-[4-(3-fluoro-benzyloxy)-phenyl]-N-methyl-butyramide;
3-[4-(3-Fluoro-benzyloxy)-phenyl]-propynoic acid methylamide;
3-[4-(3-fluoro-benzyloxy)-phenyl]-2-methyl-acrylamide;
3-[4-(3-fluoro-benzyloxy)-phenyl]-2,N-dimethyl-propionamide; and,
3-[4-(3-fluoro-benzyloxy)-phenyl]-propynoic acid amide;
or pharmaceutically acceptable salts thereof.

53. The method of claim 1, wherein the inhibitor is of formula XXX or a pharmaceutically acceptable salt thereof: 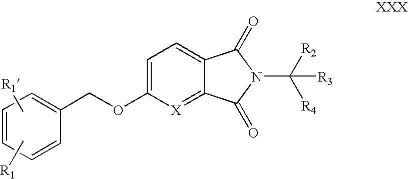
wherein:
X is selected from N and CH;
R₁ and R₁′ are independently selected from H, halogen, C₁₋₆ alkyl, C₁₋₆ haloalkyl, —CN, C₁₋₆ alkoxy, C₁₋₆ haloalkoxy, and CF₃;
R₂ is selected from H, (CH₂)ₙCN, (CH₂)ₙOR₄, (CH₂)ₙCON(R₄)₂, (CH₂)ₙCO₂R₄, CHR₅(CH₂)ₙOR₆, and (CH₂)ₙN(R₄)₂;
R₃ is selected from H, C₁₋₆ alkyl, OH, (CH₂)ₙO—C₁₋₆ alkyl, (CH₂)ₙS—C₁₋₆ alkyl, (CH₂)ₙS(O)—C₁₋₆ alkyl, benzyl, and C₁₋₆ haloalkoxy;
R₄ is selected from H and C₁₋₆ alkyl;
R₅ is selected from H, C₁₋₆ alkyl, —CN, and CONH₂;
R₆ is selected from H and C₁₋₆ alkyl; and,
n is selected from 0, 1, 2, and 3.

54. The method of claim 53, wherein the inhibitor is selected from:
(S)-2-[5-(4-Fluoro-benzyloxy)-1,3-dioxo-1,3-dihydro-isoindol-2-yl]-propionamide;
2-(2-Amino-ethyl)-5-(4-fluoro-benzyloxy)-isoindole-1,3-dione 1:1 hydrochloride;
4-[5-(4-Fluoro-benzyloxy)-1,3-dioxo-1,3-dihydro-isoindol-2-yl]-3-hydroxy-butyramide;
(S)-5-(3-Methoxy-benzyloxy)-2-(2-methoxy-1-methyl-ethyl)-isoindole-1,3-dione;
(S)-4-[2-(2-Methoxy-1-methyl-ethyl)-1,3-dioxo-2,3-dihydro-1H-isoindol-5-yloxymethyl]-benzonitrile;
2-(2-Ethanesulfinyl-ethyl)-5-(4-fluoro-benzyloxy)-isoindole-1,3-dione;
(S)-2-(2-Methoxy-1-methyl-ethyl)-5-(4-trifluoromethoxy-benzyloxy)-isoindole-1,3-dione;
5-(4-Fluoro-benzyloxy)-2-thiophen-2-ylmethyl-isoindole-1,3-dione;
2-(2-Ethanesulfinyl-ethyl)-5-(4-fluoro-benzyloxy)-isoindole-1,3-dione;
(5-(4-Fluoro-benzyloxy)-2-(3,3,3-trifluoro-2-methoxy-propyl)-isoindole-1,3-dione;
(S)-2-[5-(3-Fluoro-benzyloxy)-1,3-dioxo-1,3-dihydro-isoindol-2-yl]-propionamide;
2-(2-Amino-ethyl)-5-(3-fluoro-benzyloxy)-isoindole-1,3-dione 1:1 hydrochloride;
2-[5-(4-fluoro-benzyloxy)-1,3-dioxo-1,3-dihydro-isoindol-2-yl]-acetamide;
(S)-2-[5-(4-fluoro-benzyloxy)-1,3-dioxo-1,3-dihydro-isoindol-2-yl]-propionamide;
(S)-2-[5-(4-fluoro-benzyloxy)-1,3-dioxo-1,3-dihydro-isoindol-2-yl]-3-hydroxy-propionamide;
(R)-2-[5-(4-fluoro-benzyloxy)-1,3-dioxo-1,3-dihydro-isoindol-2-yl]-propionamide;
2-[5-(3-fluoro-benzyloxy)-1,3-dioxo-1,3-dihydro-isoindol-2-yl]-propionamide;
(2-[5-(3-fluoro-benzyloxy)-1,3-dioxo-1,3-dihydro-isoindol-2-yl]-acetamide;
2-[5-(3-fluoro-benzyloxy)-1,3-dioxo-1,3-dihydro-isoindol-2-yl]-3-hydroxy-propionamide;
5-(4-fluoro-benzyloxy)-2-(2-hydroxy-ethyl)-isoindole-1,3-dione;
5-(4-fluoro-benzyloxy)-2-(2-methoxy-ethyl)-isoindole-1,3-dione;
5-(3-fluoro-benzyloxy)-2-(2-methoxy-ethyl)-isoindole-1,3-dione;
(S)-5-(4-fluoro-benzyloxy)-2-(2-methoxy-1-methyl-ethyl)-isoindole-1,3-dione;
(S)-5-(3-fluoro-benzyloxy)-2-(2-methoxy-1-methyl-ethyl)-isoindole-1,3-dione;
(S)-5-(2-fluoro-benzyloxy)-2-(2-methoxy-1-methyl-ethyl)-isoindole-1,3-dione;
(S)-2-(2-methoxy-1-methyl-ethyl)-5-(4-trifluoromethyl-benzyloxy)-isoindole-1,3-dione;
(S)-5-(4-bromo-benzyloxy)-2-(2-methoxy-1-methyl-ethyl)-isoindole-1,3-dione;
(S)-5-(3,4-difluoro-benzyloxy)-2-(2-methoxy-1-methyl-ethyl)-isoindole-1,3-dione;
5-(3-fluoro-benzyloxy)-2-(2-hydroxy-ethyl)-isoindole-1,3-dione;
5-(4-fluoro-benzyloxy)-2-(3,3,3-trifluoro-2-hydroxy-propyl)-isoindole-1,3-dione;
5-(3,5-bis-trifluoromethyl-benzyloxy)-2-(2-methoxy-1-methyl-ethyl)-isoindole-1,3-dione;
2-(2-ethylsulfanyl-ethyl)-5-(4-fluoro-benzyloxy)-isoindole-1,3-dione;
(S)-2-[5-(4-fluoro-benzyloxy)-1,3-dioxo-1,3-dihydro-isoindol-2-yl]-thiopropionamide;
2-(2-ethylsulfanyl-ethyl)-5-(3-fluoro-benzyloxy)-isoindole-1,3-dione;
5-(4-fluoro-benzyloxy)-1,3-dioxo-1,3-dihydro-isoindol-2-yl]-acetonitrile; and,
[5-(3-fluoro-benzyloxy)-1,3-dioxo-1,3-dihydro-isoindol-2-yl]-acetonitrile;
or pharmaceutically acceptable salts thereof.

55. The method of claim 1, wherein the inhibitor is of formula XXXI or a pharmaceutically acceptable salt thereof: 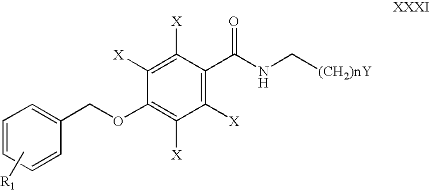
wherein:
X is selected from H and F;
Y is selected from NH₂, —CN, OH, C₁₋₆ alkoxy, and CON(R₂)₂;
R₁ is selected from H, halogen, C₁₋₆ alkyl, C₁₋₆ haloalkyl, —CN, C₁₋₆ alkoxy, and C₁₋₆ haloalkoxy; and,
n is selected from 0, 1, and 2.

56. The method of claim 55, wherein the inhibitor is selected from:
(S)-N-(1-carbamoyl-ethyl)-2-fluoro-4-(3-fluoro-benzyloxy)-benzamide;
2-[4-(3-fluorobenzyloxy)-2-fluoro-benzamido]acetamide;
(S)-N-(1-carbamoyl-2-hydroxy-ethyl)-2-fluoro-4-(3-fluoro-benzyloxy)-benzamide;
(R)-N-(1-carbamoyl-ethyl)-2-fluoro-4-(3-fluoro-benzyloxy)-benzamide;
2-[4-(4-fluorobenzyloxy)-2-fluoro-benzamido]acetamide;
(S)-N-(1-carbamoyl-ethyl)-2-fluoro-4-(4-fluoro-benzyloxy)-benzamide;
S)-2-Fluoro-4-(3-fluoro-benzyloxy)-N-(2-methoxy-1-methyl-ethyl)-benzamide;
2-fluoro-4-(3-fluoro-benzyloxy)-N-(2-methoxy-ethyl)-benzamide;
2-fluoro-4-(3-fluoro-benzyloxy)-N-(2-hydroxy-ethyl)-benzamide;
S)-N-(1-carbamoyl-ethyl)-3-fluoro-4-(4-fluoro-benzyloxy)-benzamide;
2-[4-(4-fluorobenzyloxy)-3-fluoro-benzamido]acetamide;
(S)-N-(1-carbamoyl-2-hydroxy-ethyl)-3-fluoro-4-(4-fluoro-benzyloxy)-benzamide;
2-[4-(3-fluorobenzyloxy)-3-fluoro-benzamido]acetamide;
(S)-N-(1-carbamoyl-ethyl)-3-fluoro-4-(3-fluoro-benzyloxy)-benzamide;
(R)-N-(1-carbamoyl-ethyl)-3-fluoro-4-(3-fluoro-benzyloxy)-benzamide;
(S)-N-(1-carbamoyl-2-hydroxy-ethyl)-3-fluoro-4-(3-fluoro-benzyloxy)-benzamide;
2-[4-(4-trifluoromethylbenzyloxy)-3-fluoro-benzamido]acetamide;
(S)-N-(1-carbamoyl-2-hydroxy-ethyl)-3-fluoro-4-(4-trifluoromethyl-benzyloxy)-benzamide;
(S)-N-(1-carbamoyl-ethyl)-2,6-difluoro-4-(4-fluoro-benzyloxy)-benzamide;
N-carbamoylmethyl-2,6-difluoro-4-(4-fluoro-benzyloxy)-benzamide;
N-cyanomethyl-2,6-difluoro-4-(4-fluoro-benzyloxy)-benzamide;
2,6-difluoro-4-(4-fluoro-benzyloxy)-N-(2-methoxy-ethyl)-benzamide;
(S)-2,6-difluoro-4-(4-fluoro-benzyloxy)-N-(2-hydroxy-1-methyl-ethyl)-benzamide; and,
2,6-difluoro-4-(3-fluoro-benzyloxy)-N-(2-methoxy-ethyl)-benzamide;
or pharmaceutically acceptable salts thereof.

57. The method of claim 1, wherein the inhibitor is of formula XXXII or stereoisomers or a pharmaceutically acceptable salt thereof:

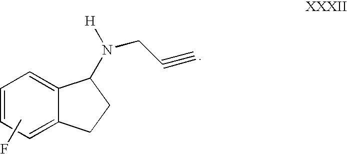

58. The method of claim 57, wherein the inhibitor is selected from:
4-fluoro-N-propargyl-1-aminoindan;
5-fluoro-N-propargyl-1-aminoindan;
6-fluoro-N-propargyl-1-aminoindan;
(+)-6-fluoro-N-propargyl-1-aminoindan; and,
or stereoisomers or pharmaceutically acceptable salts thereof.

59. The method of claim 1, wherein the inhibitor is of formula XXXIII or a pharmaceutically acceptable salt thereof: 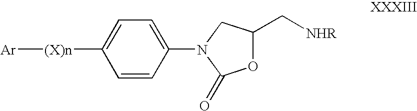
wherein:
X is selected from —CH₂CH₂—, —CH═CH—, —C≡C—, and —CH₂O—, where the CH₂ portion of CH₂O is linked to Ar;
Ar is selected from phenyl; phenyl substituted by a halogen atom or CF₃, and 3-chloro-4-fluoro-phenyl;
R is selected from H, C₁₋₆ alkyl, C₁₋₆ alkenyl, and C₁₋₆ alkynyl; and,
n is selected from 1, 2, and 3.

60. The method of claim 59, wherein the inhibitor is selected from:
5-Aminomethyl-3-{4-[2-(3-chloro-phenyl)-ethyl]-phenyl}-oxazolidin-2-one;
5-Aminomethyl-3-{4-[2-(3-chloro-phenyl)-vinyl]-phenyl}-oxazolidin-2-one;
5-Aminomethyl-3-{4-[2-(3-fluoro-phenyl)-ethyl]-phenyl}-oxazolidin-2-one;
3-{4-[2-(3-Fluoro-phenyl)-ethyl]-phenyl}-5-methylaminomethyl-oxazolidin-2-one;
3-{4-[2-(3-Chloro-phenyl)-vinyl]-phenyl}-5-methylaminomethyl-oxazolidin-2-one;
3-[4-(3-Chloro-benzyloxy)-phenyl]-5-propylaminomethyl-oxazolidin-2-one; and,
3-[4-(3-Chloro-benzyloxy)-phenyl]-5-ethynylaminomethyl-oxazolidin-2-one;
or pharmaceutically acceptable salts thereof.

61. The method of claim 1, wherein the inhibitor is of formula XXXIV or stereoisomers or a pharmaceutically acceptable salt thereof: 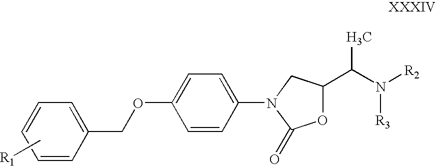
wherein:
R₁ is selected from H, C₁₋₄ alkoxy, CF₃, and one or two halogen atoms; and
R₂ and R₃ are independently selected from H and C₁₋₄ alkyl.

62. The method of claim 61, wherein the inhibitor is selected from:
3-[4-(3-chlorobenzyloxy)phenyl]-5-(1-dimethylaminoethyl)-oxazolidin-2-one;
3-[4-(3-methoxybenzyloxy)phenyl]-5-(1-dimethylaminoethyl)-oxazolidin-2-one;
3-[4-(3-chlorobenzyloxy)phenyl]-5-(1-methylaminoethyl)-oxazolidin-2-one;
3-[4-(3-methoxybenzyloxy)phenyl]-5-(1-methylaminoethyl)-oxazolidin-2-one;
3-[4-(3-chlorobenzyloxy)phenyl]-5-(1-aminoethyl)-oxazolidin-2-one; and,
3-[4-(3-methoxybenzyloxy)phenyl]-5-(1-aminoethyl)-oxazolidin-2-one;
or stereoisomers or pharmaceutically acceptable salts thereof.

63. The method of claim 1, wherein the inhibitor is of formula XXXV or a pharmaceutically acceptable salt thereof: 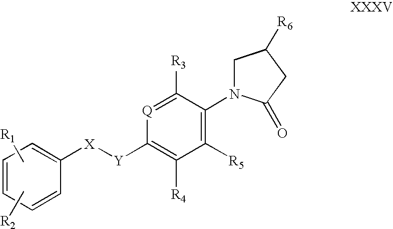
wherein:
Q is selected from N or CR₇;
X—Y is selected from —CH₂CH₂—, —CH═CH—, and —CH₂—;
R₁ and R₂ are selected from H, halogen, —CN, C₁₋₆ alkyl, C₁₋₆ haloalkyl, —CN, C₁₋₆ alkoxy, and C₁₋₆ haloalkoxy;
R₃, R₄, and R₅ are selected from H and halogen;
R₇ is selected from H, halogen, and methyl;
R₆ is selected from NHR₉, CONHR, —CN, and CH₂CN; and,
R₉ is selected from C(O)H, C(O)C₁₋₃-alkyl, C(O)-halo-C₁₋₃ alkyl, C(O)OC₁₋₃-alkyl, CONH₂, and SO₂—C₁₋₃-alkyl.

64. The method of claim 63, wherein the inhibitor is selected from:
(RS)-1-[4-(3-fluoro-benzyloxy)-phenyl]-5-oxo-pyrrolidine-3-carboxylic acid methylamide,
(RS)-[1-[4-(4-fluoro-benzyloxy)-phenyl]-5-oxo-pyrrolidine-3-carboxylic acid methylamide,
(RS)-1-[4-(3-chloro-benzyloxy)-phenyl]-5-oxo-pyrrolidine-3-carboxylic acid methylamide,
(RS)-[1-[4-(3,4-difluoro-benzyloxy)-phenyl]-5-oxo-pyrrolidine-3-carboxylic acid methylamide,
(RS)-[1-[4-(2,6-difluoro-benzyloxy)-phenyl]-5-oxo-pyrrolidine-3-carboxylic acid methylamide,
(RS)-5-oxo-1-[4-(2,4,6-trifluoro-benzyloxy)-phenyl]-pyrrolidine-3-carboxylic acid methylamide,
(RS)-5-oxo-1-[4-(2,4,5-trifluoro-benzyloxy)-phenyl]-pyrrolidine-3-carboxylic acid methylamide,
(RS)-5-oxo-1-[4-(2,3,6-trifluoro-benzyloxy)-phenyl]-pyrrolidine-3-carboxylic acid methylamide,
(RS)-5-oxo-1-[4-(2,3,4-trifluoro-benzyloxy)-phenyl]-pyrrolidine-3-carboxylic acid methylamide,
(RS)-5-oxo-1-[4-(3,4,5-trifluoro-benzyloxy)-phenyl]-pyrrolidine-3-carboxylic acid methylamide.
(RS)-1-[4-(5-fluoro-2-methyl-benzyloxy)-phenyl]-5-oxo-pyrrolidine-3-carboxylic acid methylamide,
(RS)-1-[4-(3-methoxy-benzyloxy)-phenyl]-5-oxo-pyrrolidine-3-carboxylic acid methylamide,
(RS)-1-[4-(2-methoxy-benzyloxy)-phenyl]-5-oxo-pyrrolidine-3-carboxylic acid methylamide,
(RS)-5-oxo-1-[4-(3-trifluoromethoxy-benzyloxy)-phenyl]-pyrrolidine-3-carboxylic acid methylamide,
(RS)-5-oxo-1-[4-(3-trifluoromethyl-benzyloxy)-phenyl]-pyrrolidine-3-carboxylic acid methylamide,
(RS)-1-[4-(3-cyano-benzyloxy)-phenyl]-5-oxo-pyrrolidine-3-carboxylic acid methylamide,
(RS)-1-[4-(3-fluoro-benzyloxy)-3-methyl-phenyl]-5-oxo-pyrrolidine-3-carboxylic acid methylamide,
(RS)-1-[4-(4-fluoro-benzyloxy)-3-methyl-phenyl]-5-oxo-pyrrolidine-3-carboxylic acid methylamide,
(RS)-1-[4-(3-chloro-benzyloxy)-3-methyl-phenyl]-5-oxo-pyrrolidine-3-carboxylic acid methylamide,
(RS)-1-[3-fluoro-4-(3-fluoro-benzyloxy)-phenyl]-5-oxo-pyrrolidine-3-carboxylic acid methylamide,
(RS)-1-[2-fluoro-4-(3-fluoro-benzyloxy)-phenyl]-5-oxo-pyrrolidine-3-carboxylic acid methylamide,
(RS)-1-[2,5-difluoro-4-(3-fluoro-benzyloxy)-phenyl]-5-oxo-pyrrolidine-3-carboxylic acid methylamide
(RS)-1-(4-benzyloxy-phenyl)-5-oxo-pyrrolidine-3-carboxylic acid methylamide,
(R)-1-[4-(3-fluoro-benzyloxy)-phenyl]-5-oxo-pyrrolidine-3-carboxylic acid methylamide,
(S)-1-[4-(3-fluoro-benzyloxy)-phenyl]-5-oxo-pyrrolidine-3-carboxylic acid methylamide,
(R)-1-(4-benzyloxy-phenyl)-5-oxo-pyrrolidine-3-carboxylic acid methylamide,
(S)-1-(4-benzyloxy-phenyl)-5-oxo-pyrrolidine-3-carboxylic acid methylamide,
(R)-1-[4-(4-fluoro-benzyloxy)-phenyl]-5-oxo-pyrrolidine-3-carboxylic acid methylamide,
(R)-1-[4-(3-fluoro-benzyloxy)-phenyl]-5-oxo-pyrrolidine-3-carboxylic acid methylamide,
(R)-1-[4-(3-chloro-benzyloxy)-phenyl]-5-oxo-pyrrolidine-3-carboxylic acid methylamide,
(R)-1-[4-(2,6-difluoro-benzyloxy)-phenyl]-5-oxo-pyrrolidine-3-carboxylic acid methylamide
(R)-5-oxo-1-[4-(2,4,6-trifluoro-benzyloxy)-phenyl]-pyrrolidine-3-carboxylic acid methylamide,
(RS)-1-[4-(3,4-difluoro-benzyloxy)-phenyl]-5-oxo-pyrrolidin-3-yl}-acetonitrile,
(RS)-{1-[4-(3-fluoro-benzyloxy)-phenyl]-5-oxo-pyrrolidin-3-yl}-acetonitrile,
(RS)-[1-(4-benzyloxy-phenyl)-5-oxo-pyrrolidin-3-yl]-acetonitrile,
(RS)-(E)-1-{4-[2-(3-fluoro-phenyl)-vinyl]-phenyl}-5-oxo-pyrrolidine-3-carboxylic acid methylamide,
(RS)-(E)-1-{4-[2-(4-methoxy-phenyl)-vinyl]-phenyl}-5-oxo-pyrrolidine-3-carboxylic acid methylamide,
(RS)-(E)-1-{4-[2-(3-methoxy-phenyl)-vinyl]-phenyl}-5-oxo-pyrrolidine-3-carboxylic acid methylamide,
(RS)-(E)-1-{4-[2-(4-fluoro-phenyl)-vinyl]-phenyl}-5-oxo-pyrrolidine-3-carboxylic acid methylamide
(RS)-1-{4-[2-(3-chloro-phenyl)-ethyl]-phenyl}-5-oxo-pyrrolidine-3-carboxylic acid methylamide,
(RS)-1-{4-[2-(4-chloro-phenyl)-ethyl]-phenyl}-5-oxo-pyrrolidine-3-carboxylic acid methylamide,
(RS)-1-{4-[2-(3-fluoro-phenyl)-ethyl]-phenyl}-5-oxo-pyrrolidine-3-carboxylic acid methylamide,
(RS)-1-{4-[2-(4-fluoro-phenyl)-ethyl]-phenyl}-5-oxo-pyrrolidine-3-carboxylic acid methylamide,
(RS)-1-{4-[2-(3-methoxy-phenyl)-ethyl]-phenyl}-5-oxo-pyrrolidine-3-carboxylic acid methylamide,
(RS)-1-[6-(4-fluoro-benzyloxy)-pyridin-3-yl]-5-oxo-pyrrolidine-3-carboxylic acid methylamide,
(RS)-1-[4-(2-fluoro-benzyloxy)-phenyl]-5-oxo-pyrrolidine-3-carboxylic acid methylamide,
(RS)-N-{1-[4-(3-fluoro-benzyloxy)-phenyl]-5-oxo-pyrrolidin-3-yl}-acetamide,
(S)-N-{1-[4-(3-fluoro-benzyloxy)-phenyl]-5-oxo-pyrrolidin-3-yl}-acetamide,
(R)-N-{1-[4-(3-fluoro-benzyloxy)-phenyl]-5-oxo-pyrrolidin-3-yl}-acetamide,
(RS)-N-{1-[4-(3-fluoro-benzyloxy)-phenyl]-5-oxo-pyrrolidin-3-yl}-formamide,
(S)-N-{1-[4-(3-fluoro-benzyloxy)-phenyl]-5-oxo-pyrrolidin-3-yl}-formamide,
(R)-N-{1-[4-(3-fluoro-benzyloxy)-phenyl]-5-oxo-pyrrolidin-3-yl}-formamide,
(RS)-{1-[4-(3-fluoro-benzyloxy)-phenyl]-5-oxo-pyrrolidin-3-yl}-carbamic acid methyl ester,
(RS)-{1-[4-(3-fluoro-benzyloxy)-phenyl]-5-oxo-pyrrolidin-3-yl}-urea,
(RS)-N-{1-[4-(3-fluoro-benzyloxy)-phenyl]-5-oxo-pyrrolidin-3-yl}-methanesulfonamide,
(S)-2-fluoro-N-{1-[4-(3-fluoro-benzyloxy)-phenyl]-5-oxo-pyrrolidin-3-yl}-acetamide,
(S)-2,2-difluoro-N-{1-[4-(3-fluoro-benzyloxy)-phenyl]-5-oxo-pyrrolidin-3-yl}-acetamide,
(S)-2,2,2-trifluoro-N-{1-[4-(3-fluoro-benzyloxy)-phenyl]-5-oxo-pyrrolidin-3-yl}-acetamide,
(RS)-N-{1-[4-(4-fluoro-benzyloxy)-phenyl]-5-oxo-pyrrolidin-3-yl}-acetamide,
(R)-N-{1-[4-(4-fluoro-benzyloxy)-phenyl]-5-oxo-pyrrolidin-3-yl}-acetamide,
(S)-N-{1-[4-(4-fluoro-benzyloxy)-phenyl]-5-oxo-pyrrolidin-3-yl}-acetamide,
(RS)-N-{1-[4-(4-fluoro-benzyloxy)-phenyl]-5-oxo-pyrrolidin-3-yl}-formamide,
(RS)-N-[1-(4-benzyloxy-phenyl)-5-oxo-pyrrolidin-3-yl]-acetamide,
(RS)-N-{1-[4-(2-fluoro-benzyloxy-phenyl]-5-oxo-pyrrolidin-3-yl}-acetamide,
(RS)-(E)-N-(1-{4-[2-(3-fluoro-phenyl)-vinyl]-phenyl}-5-oxo-pyrrolidin-3-yl)-acetamide
(RS)-N-(1-{4-[2-(3-fluoro-phenyl)-ethyl]-phenyl}-5-oxo-pyrrolidin-3-yl)-acetamide,
(RS)-N-{1-[6-(4-fluoro-benzyloxy)-pyridin-3-yl]-5-oxo-pyrrolidin-3-yl}-acetamide,
(S)-N-{1-[4-(3-chloro-benzyloxy)-phenyl]-5-oxo-pyrrolidin-3-yl}-acetamide,
(S)-N-{1-[4-(2,6-difluoro-benzyloxy)-phenyl]-5-oxo-pyrrolidin-3-yl}acetamide,
(S)-N-{5-oxo-1-[4-(2,4,6-trifluoro-benzyloxy)-phenyl]-pyrrolidin-3-yl}-acetamide,
(S)-N-{1-[4-(3-methoxy-benzyloxy)-phenyl]-5-oxo-pyrrolidin-3-yl}-acetamide,
(S)-N-{5-oxo-1-[4-(4-trifluoromethyl-benzyloxy)-phenyl]-pyrrolidin-3-yl}-acetamide,
(S)-N-{1-[4-(4-methyl-benzyloxy)-phenyl]-5-oxo-pyrrolidin-3-yl}-acetamide,
(S)-N-{1-[4-(3-cyano-benzyloxy)-phenyl]-5-oxo-pyrrolidin-3-yl}-acetamide.
(RS)-1-(4-benzyloxy-phenyl)-2-oxo-pyrrolidine-3-carbonitrile,
(RS)-1-[4-(3-fluoro-benzyloxy)-phenyl]-2-oxo-pyrrolidine-3-carboxylic acid methylamide,
(RS)-1-[4-(3-fluoro-benzyloxy)-phenyl]-2-oxo-pyrrolidine-3-carboxylic acid amide,
(RS)-1-[4-(4-fluoro-benzyloxy)-phenyl]-2-oxo-pyrrolidine-3-carboxylic acid amide,
(RS)-1-[4-(4-fluoro-benzyloxy)-phenyl]-2-oxo-pyrrolidine-3-carboxylic acid methylamide,
(RS)-2-oxo-1-[4-(4-trifluoromethyl-benzyloxy)-phenyl]-pyrrolidine-3-carboxylic acid amide,
(RS)-2-oxo-1-[4-(4-trifluoromethyl-benzyloxy)-phenyl]-pyrrolidine-3-carboxylic acid methylamide,
(S)-N-[1-(4-benzyloxy-phenyl)-2-oxo-pyrrolidin-3-yl]-acetamide,
(S)-N-[1-(4-benzyloxy-phenyl)-2-oxo-pyrrolidin-3-yl]-methanesulfonamide,
(S)-N-{1-[4-(3-fluoro-benzyloxy)-phenyl]-2-oxo-pyrrolidin-3-yl}-acetamide,
(R)-N-{1-[4-(3-fluoro-benzyloxy)-phenyl]-2-oxo-pyrrolidin-3-yl}-acetamide,
(R)-N-{1-[4-(3-fluoro-benzyloxy)-phenyl]-2-oxo-pyrrolidin-3-yl}-methanesulfonamide,
(S)-N-{1-[4-(3-fluoro-benzyloxy)-phenyl]-2-oxo-pyrrolidin-3-yl}-methanesulfonamide,
(S)-{1-[4-(3-fluoro-benzyloxy)-phenyl]-2-oxo-pyrrolidin-3-yl}-carbamic acid methyl ester,
(R)-N-{1-[4-(3-fluoro-benzyloxy)-phenyl]-2-oxo-pyrrolidin-3-yl}-formamide,
(S)-N-{1-[4-(3-fluoro-benzyloxy)-phenyl]-2-oxo-pyrrolidin-3-yl}-formamide,
(R)-{1-[4-(3-fluoro-benzyloxy)-phenyl]-2-oxo-pyrrolidin-3-yl}-urea,
(S)-{1-[4-(3-fluoro-benzyloxy)-phenyl]-2-oxo-pyrrolidin-3-yl}-urea,
(S)-N-{1-(S)-[4-(4-fluoro-benzyloxy)-phenyl]-2-oxo-pyrrolidin-3-yl}-acetamide,
(S)-N-{1-(S)-[4-(2,6-difluoro-benzyloxy)-phenyl]-2-oxo-pyrrolidin-3-yl}-acetamide, and
(S)-N-{1-[4-(3,4-difluoro-benzyloxy)-phenyl]-2-oxo-pyrrolidin-3-yl}-acetamide,
or pharmaceutically acceptable salts thereof.

65. The method of claim 1, wherein the inhibitor is of formula XXXVI or a pharmaceutically acceptable salt thereof: 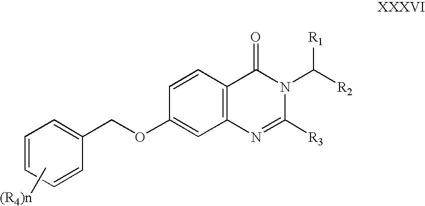
wherein:
R₁ is selected from (CH₂)ₙCONR₅R₆, (CH₂)ₙCOOR₇, (CH₂)ₙNR₅R₆, (CH₂)ₙCN, (CH₂)ₙOR₈, and phenyl that is unsubstituted or substituted by 1-3 substituents selected from halogen and fluoro-C₁₋₆ alkyl;
R₂ is selected from H, C₁₋₆ alkyl, and C₃₆-cycloalkyl;
R₃ is selected from H, C₁₋₆ alkyl, C₃₋₆ cycloalkyl, and benzyl;
R₄ is selected from halogen, cyano, C₁₋₆ alkyl, C₁₋₆ fluorooalkyl, —CN, C₁₋₆ alkoxy, and C₁₋₆ fluorooalkoxy;
R₅ and R₆ are independently selected from H and C₁₋₆ alkyl;
R₇ is selected from H and C₁₋₆ alkyl;
R₈ is C₁₋₆ alkyl;
m is selected from 1, 2 and 3; and,
n is selected from 0, 1, 2.

66. The method of claim 65, wherein the inhibitor is selected from:
2-[7-(3-fluoro-benzyloxy)-4-oxo-4H-quinazolin-3-yl]-acetamide,
2-[7-(3-fluoro-benzyloxy)-4-oxo-4H-quinazolin-3-yl]-propionamide,
2-[7-(4-fluoro-benzyloxy)-4-oxo-4H-quinazolin-3-yl]-acetamide,
2-[7-(4-fluoro-benzyloxy)-4-oxo-4H-quinazolin-3-yl]-propionamide,
2-[7-(3-fluoro-benzyloxy)-2-methyl-4-oxo-4H-quinazolin-3-yl]-acetamide,
2-[2-cyclopropyl-7-(3-fluoro-benzyloxy)-4-oxo-4H-quinazolin-3-yl]-acetamide,
7-(3-fluoro-benzyloxy)-3-(2-methoxy-ethyl)-3H-quinazolin-4-one,
7-(4-fluoro-benzyloxy)-3-(2-methoxy-ethyl)-3H-quinazolin-4-one,
7-(3-fluoro-benzyloxy)-3-(2-methoxy-ethyl)-2-methyl-3H-quinazolin-4-one,
3-(2-amino-ethyl)-7-(3-fluoro-benzyloxy)-3H-quinazolin-4-one 1:2 hydrochloride,
3-(3-amino-propyl)-7-(3-fluoro-benzyloxy)-3H-quinazolin-4-one 1:2 hydrochloride,
3-(2-amino-ethyl)-7-(4-fluoro-benzyloxy)-3H-quinazolin-4-one 1:1 hydrochloride,
2-[7-(3-fluoro-benzyloxy)-2-methyl-4-oxo-4H-quinazolin-3-yl]-ethyl-ammonium chloride,
[7-(3-fluoro-benzyloxy)-4-oxo-4H-quinazolin-3-yl]-acetic acid ethyl ester; fluoro-[7-(3-fluoro-benzyloxy)-4-oxo-4H-quinazolin-3-yl]-acetic acid ethyl ester;
2-[7-(3-fluoro-benzyloxy)-4-oxo-4H-quinazolin-3-yl]-propionic acid ethyl ester;
[7-(3-fluoro-benzyloxy)-4-oxo-4H-quinazolin-3-yl]-acetic acid tert-butyl ester;
2-[7-(3-fluoro-benzyloxy)-4-oxo-4H-quinazolin-3-yl]-propionic acid tert-butyl ester;
[7-(4-fluoro-benzyloxy)-4-oxo-4H-quinazolin-3-yl]-acetic acid ethyl ester;
2-[7-(4-fluoro-benzyloxy)-4-oxo-4H-quinazolin-3-yl]-propionic acid ethyl ester,
3-(3-fluoro-benzyl)-7-(3-fluoro-benzyloxy)-3H-quinazolin-4-one;
3-[7-(4-fluoro-benzyloxy)-4-oxo-4H-quinazolin-3-yl]-propionamide;
2-[7-(3-fluoro-benzyloxy)-2-isopropyl-4-oxo-4H-quinazolin-3-yl]-acetamide;
[7-(3-fluoro-benzyloxy)-2-isopropyl-4-oxo-4H-quinazolin-3-yl]-acetonitrile;
2-cyclopropyl-7-(3-fluoro-benzyloxy)-3-(2-methoxy-ethyl)-3H-quinazolin-4-one;
[2-cyclopropyl-7-(3-fluoro-benzyloxy)-4-oxo-4H-quinazolin-3-yl]-acetic acid methyl ester; and
2-[2-benzyl-7-(3-fluoro-benzyloxy)-4-oxo-4H-quinazolin-3-yl]-acetamide, or pharmaceutically acceptable salts thereof.

67. The method of claim 1, wherein the inhibitor is of formula XXXVII or a pharmaceutically acceptable salt thereof: 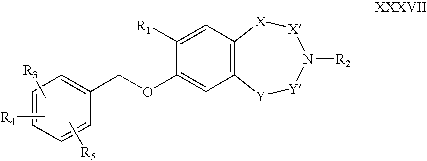
wherein:
R₁ is selected from H and methyl;
R₂ is selected from H, C₁₋₃ alkyl, CH₂CONH₂, CH(CH₃)CONH₂, SO₂CH₃, and COR₆;
R₃, R₄, and R₅ are independently selected from H, halogen, —CN, C₁₋₃ alkyl, and C₁₋₃ alkoxy;
R₆ is selected from H, methyl, CH₂OCH₃, CONH₂, CH₂CONH₂, OCH₃, NH₂, and NHCH₂CH₃;
X—X′ is selected from —CH₂CH₂—, —CH═CH—, and —CH₂CO—;
Y—Y′ is selected from —CH₂CH₂, —CH═CH—, and —CH₂CO—;
X—X′ is selected from —CH₂—, and Y—Y′ is CH₂CH₂CO—; provided that:
when one of X—X′ and Y—Y′ is —CH₂CH₂— and the other is —CH═CH;
when both of X—X′ and Y—Y′ are —CH=CH—, then R₂ is SO₂CH₃ or —COR₆; or
when X—X′ and Y—Y′ are —CH₂CO—, then R₂ is H, C₁₋₃ alkyl, CH₂CONH₂, or
CH(CH₃)CONH₂.

68. The method of claim 67, wherein the inhibitor is selected from:
1-[7-(3-fluoro-benzyloxy)-1,2,4,5-tetrahydro-benzo[d]azepin-3-yl]-ethanon-e,
1-[7-(3-fluoro-benzyloxy)-1,2,4,5-tetrahydro-benzo[d]azepin-3-yl]-2-met-hoxy-ethanone,
2-[7-(3-fluoro-benzyloxy)-1,2,4,5-tetrahydro-benzo[d]azepin-3-yl]-2-oxo-acetamide,
3-[7-(3-fluoro-benzyloxy)-1,2,4,5-tetrahydro-benzo-[d]azepin-3-yl]-3-oxo-propionamide,
7-(3-fluoro-benzyloxy)-1,2,4,5-tetrahydro-benzo[d]azepine-3-carboxylic acid methyl ester,
7-(3-fluoro-benzyloxy)-1,2,4,5-tetrahydro-benzo[d]azepine-3-carbaldehyde,
7-(3-fluoro-benzyloxy)-3-methanesulfonyl-2,3,4,5-tetrahydro-1H-benzo[d]az-epine,
7-(3-fluoro-benzyloxy)-1,2,4,5-tetrahydro-benzo[d]azepine-3-carboxy-lic acid amide,
7-(3-fluoro-benzyloxy)-1,2,4,5-tetrahydro-benzo[d]azep-ine-3-carboxylic acid ethylamide.
2-[7-(3-fluoro-benzyloxy)-1,2,4,5-tetrahydro-benzo[d]azepin-3-yl]-acetami-de,
(RS)-2-[7-(3-fluoro-benzyloxy)-1,2,4,5-tetrahydro-benzo[d]azepin-3-yl]-propionamide,
8-(3-fluoro-benzyloxy)-1,3-dihydro-benzo[d]azepin-2-one,
8-(3-fluoro-benzyloxy)-3-methyl-1,3-dihydro-benzo[d]azepin-2-one,
8-(3-fluoro-benzyloxy)-3-methoxyacetyl-1,3-dihydro-benzo[d]azepin-2-one,
3-acetyl-8-(3-fluoro-benzyloxy)-1,3-dihydro-benzo[d]azepin-2-one,
8-(3-fluoro-benzyloxy)-1,3,4,5-tetrahydro-benzo[d]azepin-2-one,
7-(2,3,4-trifluoro-benzyloxy)-1,3,4,5-tetrahydro-benzo[d]azepin-2-one.
7-(2,3,4-trifluoro-benzyloxy)-1,2,4,5-tetrahydro-benzo[c]azepin-3-one,
7-(2,6-difluoro-benzyloxy)-1,3,4,5-tetrahydro-benzo[d]azepin-2-one,
7-(2,6-difluoro-benzyloxy)-1,2,4,5-tetrahydro-benzo[c]azepin-3-one,
7-benzyloxy-1,3,4,5-tetrahydro-benzo[d]azepin-2-one,
7-(3-fluoro-benzyloxy)-1,3,4,5-tetrahydro-benzo[d]azepin-2-one,
7-(3-chloro-benzyloxy)-1,3,4,5-tetrahydro-benzo[d]azepin-2-one,
3-acetyl-7-(3-chloro-benzyloxy)-1,3,4,5-tetrahydro-benzo[d]azepin-2-one, and
7-(3-fluoro-benzyloxy)-1,2,4,5-tetrahydro-benzo[c]azepin-2-one,
or pharmaceutically acceptable salts thereof.

69. The method of claim 1, wherein the inhibitor is of formula XXXVIII or pharmaceutically acceptable salts or stereoisomers thereof: 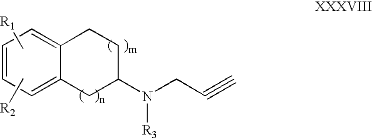
wherein:
R₁ is OH or OC(O)R₄;
R₂ is OC(O)R₄ or H;
R₃ is H or C₁₋₆ alkyl;
R₄ is selected from the group consisting of C₁₋₆ alkyl, C₆₋₁₂ aryl, C₆₋₁₂ aryl-C₁₋₆ alkylene, and, NR₅R₆;
R₅ and R₆ are independently selected from the group consisting of H, C₁₋₈ alkyl, C₆₋₁₂ aryl, C₆₋₁₂ aryl-C₁₋₆ alkylene, and C₆₋₁₂ cycloalkyl, each optionally substituted with a group selected from halogen, C₁₋₆ alkyl, C₁₋₆ alkoxy, —CN, NO₂, and OH;
n is 0 or 1; and,
m is 1 or 2.
